(19) United States
(12) Patent Application Publication
(10) Pub. No.: US 2025/0144199 A1
(43) Pub. Date: May 8, 2025

(54) COMPOSITIONS AND METHODS FOR FcRn-TARGETED INTRANASAL CORONAVIRUS VACCINATION

(71) Applicant: UNIVERSITY OF MARYLAND, COLLEGE PARK, College Park, MD (US)

(72) Inventors: Xiaoping Zhu, Clarksville, MD (US); Weizhong Li, College Park, MD (US); Tao Wang, Ellicott City, MD (US)

(73) Assignee: University of Maryland, College Park, College Park, MD (US)

(21) Appl. No.: 18/836,949

(22) PCT Filed: Feb. 9, 2023
(86) PCT No.: PCT/US2023/012712

§ 371 Date: Aug. 8, 2024

Related U.S. Application Data
(60) Provisional application No. 63/267,782, filed on Feb. 9, 2022.

(51) Int. Cl. A61K 39/215; A61P 37/04; C07K 14/005

(52) U.S. Cl. CPC A61K 39/215; A61P 37/04; C07K 14/005; C07K 2319/30; C07K 2319/40; C12N 2770/20022; C12N 2770/20034; C12N 2770/20071

(57) ABSTRACT

The present disclosure relates generally to novel recombinant coronavirus-based fusion proteins (“RBDs-IgG Fc protein” and “RBDs protein”) and vaccine compositions using the same, in which the fusion proteins comprise tandemly arranged coronaviruses receptor binding domains (RBDs). The present disclosure further provides methods and kits for immunizing a subject using the compositions.

This application claims benefit and priority to U.S. Provisional Application No. 63/267,782 filed on Feb. 9, 2022 which is incorporated herein by reference in its entirety.

### GOVERNMENT SUPPORT

This invention was made with government support under R01AI146063A awarded by the National Institutes of Health (NIH). The government has certain rights in the invention.

### TECHNICAL FIELD

The present disclosure relates generally to novel recombinant coronavirus-based fusion proteins (“RBDs-IgG Fc protein”) and vaccine compositions using the same, in which the RBDs-IgG Fc protein comprises tandemly arranged coronaviruses receptor binding domains (RBDs) fused to an IgG Fc protein domain. The present disclosure relates also to novel recombinant coronavirus-based fusion proteins (“RBDs protein”) and vaccine compositions using the same, in which the RBDs protein comprises tandemly arranged coronaviruses receptor binding domains (RBDs). Such RBDs may be derived from SARS-CoV-2, and/or variants thereof, the causal agents for COVID-19, or other coronaviruses. The present disclosure provides methods for the immunization of a subject using the vaccine compositions for treating or preventing clinical signs, infections, and transmissions caused by coronavirus infection.

### BACKGROUND

SARS-CoV-2, a virus causing the COVID-19 pandemic, is known to spread rapidly through respiratory droplets and aerosols. SARS-CoV-2 enters host cells via its receptor-binding domain (RBD) of the spike (S) binding to angiotensin-converting enzyme 2 (ACE2). ACE2 expression level shows a gradient pattern in the respiratory tract from highest in the ciliated epithelial cells of the nasal cavity, the upper bronchial epithelia, to the relatively low in type II alveolar cells of the lung. Hence, nasal ciliated cells are primary targets for SARS-CoV-2 replication in the early stage of infection. In addition, SARS-CoV-2 variants continually emerge, including Alpha (B.1.1.7), Beta (B.1.351), Gamma (P.1), Delta (B1.617.2), Omicron (B. 1.1.529), etc. These variant viruses exhibit increased transmissibility, pathogenicity, and replication, but a decreased sensitivity to neutralization by immune plasma derived from convalescent COVID-19 patients or vaccinated individuals. The mutated residues in the RBD portion, at least partially, are responsible for its higher affinity binding to ACE2, the higher infectivity, and the immune evasion of SARS-CoV-2 variants. These characteristics could explain the enduring transmissions of the SARS-CoV-2 virus during the pandemic.

To block viral transmission or shedding, vaccines should offer the potential to induce robust protective immune responses at the upper airway. In general, the secretory IgA antibody responses and tissue-resident memory T cells (TRM) in the respiratory tract can immediately prevent viral infection in the upper respiratory tract, including the nasal passages. At the present, most authorized SARS-CoV-2 vaccines are intramuscularly injected and mainly designed for inducing serum IgG, which protects the lungs and prevents viremia and the COVID-19 syndrome, but leaves the nasal epithelia largely unprotected. Hence, intramuscular vaccines generally provide limited protection against viral shedding and transmission and suggest that intramuscularly immunized individuals may still experience breakthrough infection and shed live virus from the nose. The breakthrough infection and viral transmission could be further exaggerated by the SARS-CoV-2 variants that make the currently authorized vaccines less effective. The lack of effective mucosal immunity in blocking viral spread and the emergence of variants indicate the critical need to develop a intranasal based vaccine that could induce mucosal immunity, provide broad protection against viral variants, and help reduce the viral spread.

### SUMMARY

The present disclosure relates to a recombinant coronavirus-based fusion protein (“RBDs-IgG Fc protein”) that comprises tandemly arranged coronaviruses receptor binding domains (RBDs) fused to an IgG Fc protein domain, nucleic acids encoding such RBDs-IgG Fc proteins, and their use in vaccines for production of an effective immunogenicity against the coronavirus. In a specific embodiment, the coronavirus is SARS-CoV-2. Such RBDs-IgG Fc proteins are designed to mimic antigenic sites of the viral membrane protein as an effective and immunogenic vaccine. Additionally, the present disclosure relates to a recombinant coronavirus-based fusion protein (“RBDs protein”) that comprises tandemly arranged coronaviruses receptor binding domains (RBDs), but lacking an IgG Fc protein domain, nucleic acids encoding such RBDs proteins, and their use in vaccines for production of an effective immunogenicity against the coronavirus. While the disclosure below is directed to SARS-CoV-2 based fusion proteins, it is understood that said disclosure can be applied equally as well to other coronaviruses having corresponding RBD domains.

In an embodiment, a SARS-CoV-2 based RBDs-IgG Fc protein is provided that includes one or more RBDs, or fragments thereof, arranged tandemly and fused to an IgG Fc domain. The IgG Fc domain functions to facilitate the binding of the RBDs-IgG Fc protein to Fc receptors expressed on the surface of cells for the transfer of the protein across the mucosal epithelial barrier. In an embodiment, the RBDs-IgG Fc proteins, or RBDs proteins, may be derived from identified SARS-CoV-2 variants. Such variants include, for example, alpha, beta, gamma, delta, epsilon, kappa, omicron, lota, mu and theta as well as any additional variants that may develop over time. In another non-limiting embodiment, the RBD domains may be derived from SARS-CoV-1 or MERS-related coronavirus.

The coronavirus RBD-IgG Fc protein, or RBDs protein, may further include one or more linker sequences that link the different domains of the fusion proteins. The linker sequence may be a polypeptide of 1-80 amino acids. The linker sequence may be a polypeptide of 2-50 amino acids. The linker may have a length of 1, 2, 3, 4, 5, 6, 7, 8, 9, 10, 11, 12, 13, 14, 15, 16, 17, 18, 19, 20, 21 or 22 amino acids. In an embodiment the linker sequence may be a GLY-SER amino acid linker. In an embodiment, the linker may comprise multiple GLY-SER amino acid residues. Further, a signal sequence may be included in the RBD-IgG Fc protein or RBDs protein.

Another aspect of the present disclosure pertains to nucleic acids encoding the RBD-IgG Fc proteins, or RBDs proteins, disclosed herein. Such nucleic acids may be introduced into a variety of different expression vectors, including for example, bacterial and viral expression vectors for the expression of a RBDs-IgG Fc protein, or RBDs protein, in a host cell of interest. In a specific embodiment, the nucleic acid is a cDNA or mRNA molecule capable of encoding the RBD-IgG Fc protein or RBDs protein. A nucleic acid molecule encoding an RBD-IgG Fc protein, or RBDs protein, may be chemically synthesized based on the RBD-IgG Fc protein, or RBDs protein, amino acid sequence encoded by the nucleic acid.

Recombinant expression vectors having nucleic acid molecules encoding RBD-IgG Fc proteins, or RBDs proteins, are also provided. Such recombinant expression vectors include, for example, bacterial expression vectors and eukaryotic expression vectors. Expression vectors include viral vectors such as adenovirus recombinant expression vectors. The provided nucleic acid molecules encoding RBD-IgG Fc proteins, or RBDs proteins, can be used for in vitro or in vivo gene expression of the protein for use in the prevention and/or treatment of coronavirus infection.

In still another aspect, a method is provided for preparing an RBDs-IgG Fc protein, or RBDs protein, using nucleic acids encoding the RBDs-IgG Fc protein or RBDs protein. The preparation methods according to the present disclosure may be performed through recombinant DNA or mRNA technology known in the art using a nucleic acid encoding the RBDs-IgG Fc protein or RBDs protein. This method includes, for example, (i) preparing an expression vector including a nucleic acid encoding the RBD-IgG Fc protein, or RBDs protein, of interest, (ii) transforming the expression vector into host cells of interest, and (iii) culturing the transformed host cells. In a further step, the RBDs-IgG Fc protein, or RBDs protein, may be purified from the resultant culture broth.

Also disclosed is a nanoparticle having the disclosed RBDs-IgG Fc protein, or RBDs-IgG Fc protein encoding nucleic acid. Further provided is a nanoparticle having the disclosed RBDs protein, or RBDs-IgG Fc protein encoding nucleic acid. The nanoparticles can be created from biological molecules or from non-biological molecules. In some cases, the RBDs-IgG Fc protein, or RBDs protein, or encoding nucleic acids, are crosslinked to a polymer or lipids on the nanoparticle surface. In embodiments, the RBDs-IgG Fc protein or RBDs protein, or encoding nucleic acids, are adsorbed onto the nanoparticle surface. In some embodiments, the RBDs-IgG Fc protein, or RBDs protein, or encoding nucleic acids, are adsorbed onto the nanoparticle surface and then crosslinked to the nanoparticle surface. In some embodiments, the RBDs-IgG Fc protein, or RBDs protein, or encoding nucleic acids, are encapsulated into the nanoparticle. Such nanoparticles, or nanoliposomes may be incorporated into vaccine compositions as disclosed below.

The present disclosure provides a vaccine composition containing a RBDs-IgG Fc protein, or a RBDs-IgG Fc protein encoding nucleic acid, i.e., cDNA or RNA, as an active ingredient. A vaccine composition is also provided containing a RBDs protein, or a RBDs encoding nucleic acid, i.e., cDNA or RNA, as an active ingredient. As used herein, the term “vaccine” refers to a composition able to prevent the infection or re-infection with SARS-CoV-2, reducing the severity of symptoms or eliminating symptoms by COVID-19, or substantially or completely removing COVID-19 or the disease caused by SARS-CoV-2 infection, by inducing an immune response to SARS-CoV-2 in a subject. Thus, the vaccine composition disclosed herein may be administered prophylactically to a subject, e.g., a human, before infection with SARS-CoV-2, or may be therapeutically administered to subjects after infection with SARS-CoV-2. Here, the term “immune response” includes either or both of a humoral immune response and a cellular immune response.

The vaccine composition provided herein may be prepared in any suitable and pharmaceutically acceptable formulation. It may be provided in the form of an immediately administrable solution or suspension, or a concentrated crude solution suitable for dilution before administration or may be provided in a form capable of being reconstituted, such as a lyophilized, freeze-dried, or frozen formulation. In a specific embodiment, the vaccine composition is formulated for intranasal administration.

The vaccine composition may contain a pharmaceutically acceptable carrier in order to be formulated. The carrier typically includes a diluent, an excipient, a stabilizer, a preservative, and the like. The vaccine composition of the present disclosure may further contain an adjuvant. The adjuvant may be composed of one or more substances that enhance the immune response to an antigen, e.g., the RBDs-IgG Fc protein or the RBDs protein. The adjuvant may function as a tissue reservoir that slowly releases an antigen and/or as a lymphoid system activator that nonspecifically enhances an immune response (Hood et al., Immunology, Second Ed., 1984, Benjamin/Cummings: Menlo Park, Calif., p. 384). In a specific embodiment of the invention, the vaccine composition is formulated for intranasal administration. In another embodiment, the vaccine composition is formulated for systemic administration, for example, intramuscular administration.

A method of vaccinating a subject for SARS-CoV-2 is provided that includes administering a disclosed SARS-CoV-2 vaccine composition to a subject in need thereof. The disclosed vaccine composition may be administered in a number of ways. For example, the disclosed vaccine composition can be administered intramuscularly, intranasally, orally, intravenously, subcutaneously, transdermally (e.g., by microneedle), intraperitoneally, ophthalmically, sublingually, or by inhalation. In a specific embodiment, the vaccine is administered intranasally.

The present disclosure provides a kit that includes the RBDs-IgG Fc protein vaccine compositions, or the RBDs protein vaccine compositions, as described herein. In one specific aspect the kit further includes instructions for the treatment and/or prophylaxis of COVID-19. The vaccine compositions may, if desired, be presented in a pack or dispenser device which may contain one or more-unit dosage forms containing the RBDs-IgG Fc protein vaccine composition or the RBDs protein vaccine composition. In a specific embodiment, the dispenser may be one to be used for intranasal administration of the vaccine composition. In a specific embodiment, the dispenser may be one to be used for intramuscular administration of the vaccine composition. The pack may for example include metal or plastic foil, such as a blister pack. The pack or dispenser device may be accompanied by instructions for administration to subjects, especially humans.

### BRIEF DESCRIPTION OF THE FIGURES

FIG. 1 depicts a tandem protein containing six or more RBDs of SARS-CoV-2 and its variants (B.1.1.7, B.1.351, B.1.427, P.1., and B.1.617.2)

FIG. 2 depicts a tandem RBDs protein containing six or more RBDs of SARS-CoV-2 and its variants (B.1.1.7, B.1.351, B.1.1.529, P.1., and B.1.617.2).

FIG. 3 depicts a tandem RBDs of SARS-CoV-1, SARS-CoV-2, and MERS.

FIG. 4A-B. depicts successful expression of a tandem protein comprising and/or consisting of six RBDs of SARS-CoV-2 and variants. In a coupling denaturing SDS-PAGE-Western blotting assay, it was found that the secreted proteins exhibited monomers under reducing (R) condition, but displayed dimers under non-reducing (NR) conditions (FIG. 4A). FIG. 4B demonstrates that the tandem RBDs-Fc secreted from CHO stable cell lines interacted with human ACE2 in an ELISA assay. The capacity of the tandem RBDs-Fc (RBDs-Fc and RBDs-IgG Fc are used interchangeably herein) in binding to human ACE2 was similar to that of the trimeric spike proteins from SARS-CoV-2. To show the binding specificity, as a negative control, the trimeric F-Fc or F protein from the human respiratory syncytial virus (RSV) was used in the assay.

FIG. 5A-B. demonstrates that human IgG1 Fc can bind to mouse FcRn. Balb/c mice (FIG. 5A) or human ACE2 transgenic mice (N=10-12) (FIG. 5B) were intranasally (i.n.) immunized with purified 10 μg of the tandem RBDs-Fc, or PBS along with 10 μg CpG, and boosted 2 weeks later with the same dose. The higher titers of RBD-specific IgG in sera were detected in the tandem RBD-Fc immunized mice when compared with the PBS-treated groups of mice 2 weeks after the boost (FIG. 5A-B).

FIG. 6A-D depicts mouse protection studies. In Balb/c mice infected with MA10, the majority of mice in the PBS groups had slight weight loss within 8 days after the challenge and mice losing weight up to 25% either succumbed to infection or were euthanized. In contrast, all the tandem RBDs-Fc-immunized mice had no body-weight loss (FIG. 6A). In addition, virus replicating in different tissues 5 days after the challenge (FIG. 6B) was assessed. The live virus was not detected in tissues, including nasal turbinate and lung of tandem RBDs-Fc-immunized mice. A high level of titers of the virus was detected in the nasal turbinate and lung of the PBS group (FIG. 6B). In human ACE2 transgenic mice infected with Delta virus, all mice in the PBS groups had weight loss (up to 25%) within 7-8 days after the challenge and either succumbed to infection or were euthanized (FIG. 6C). In contrast, all the tandem RBDs-Fc-immunized ACE2 transgenic mice had no body-weight loss. The tandem RBDs-Fc protein-immunized human ACE2 transgenic mice led to a full protection (FIG. 6D).

FIG. 7A-B depicts an in vitro microneutralization (MN) test performed for measuring a neutralization antibody titer. Sera from the tandem RBDs-Fc (FIG. 1) immunized mice exhibited high neutralizing activity against SARS-CoV-2 and Delta variants relative to the PBS control groups (FIG. 7A-B). Although the tandem RBDs-Fc antigen does not contain Omicron-specific RBD, a microneutralization (MN) test was also performed using the Omicron variant. It was found that after the 3^{rd} dose intranasal immunization, the majority sera from the tandem RBDs-Fc immunized mice showed neutralizing activity against Omicron variant relative to the PBS control groups, although the neutralizing activity was not detected in the sera of 3 mice (FIG. 7B).

FIG. 8A-F. Schematic illustrations of the construction of the tandem RBDs or tandem RBDs-Fc from SARS-CoV-2 variants. FIG. 8A. Schematic illustration of the full-length protein sequence of SARS-CoV-2 Spike. SP: signal peptide; NTD: N-terminal domain; RBD: receptor binding domain; TM: transmembrane domain; CT: cytoplasmic tail. FIG. 8B, 8D Diagram design of the fusion of Spike RBDs from SARS-CoV-2 variants with a Gly-Ser linker to create an RBDs fusion protein. For the purpose of the protein purification, six His residues were tagged to the carboxyl terminus of the RBDs. FIG. 8C, 8E. Diagram design of the fusion of Spike RBDs with human IgG Fc fragments to create an RBDs-Fc fusion protein. Mutations were also made in the Fc fragment by replacing Lys 322 with Ala residues to remove the complement C1q binding site. FIG. 8F. Graphic demonstration of the putative expressed RBDs-Fc based on IgG Fc dimerization and RBD proteins.

FIG. 9A-D. Expression and characterization of the tandem RBDs-Fc and RBDs. FIGS. 9A and 9B. The tandem RBDs-Fc or RBDs fusion proteins were purified from the stable CHO cell lines. The soluble RBDs-Fc (FIG. 9A) and RBDs (FIG. 9B) and proteins were purified by Protein A or anti-His affinity chromatography respectively and subjected to SDS-PAGE gel electrophoresis under reducing conditions and visualized with Coomassie blue staining. The purified tandem RBDs-Fc (FIG. 9A) or RBDs proteins were also subjected to Western blot analyses by anti-spike RBD antibody analysis. The molecular weight in kDa is marked in the left margin. FIG. 9C. Test of the tandem RBDs-Fc binding to human ACE2. The specific binding was determined by the ELISA. The soluble SARS-CoV-2 spike protein was used as a positive control, whereas respiratory syncytial virus (RSV) F protein alone or Fc-fused F proteins were used as the negative control, respectively. FIG. 9D. Determination of the tandem RBDs-Fc binding to human or mouse C1q. The specific binding to human or mouse C1q was determined by the ELISA. RBDs protein was used as a negative control, and an RSV F protein-specific monoclonal antibody (mAb, D25) was used as a positive control.

FIG. 10A-D. Interactions of the purified tandem RBDs-Fc or RBDs with SARS-CoV-2 RBD-specific human mAbs. SARS-CoV-2 RBD protein interacts with four different categories (Class 1, 2, 3, 4) of human mAbs (FIG. 10A). Interactions of the purified RBDs-Fc or RBDs with a set of SARS-CoV-2 RBD-specific mAbs (FIG. 10B, 10C, 10D). The specific binding was detected by the ELISA method. Human IgG was used as a negative control.

FIG. 11A-B. Antibody induced by intranasal (i.n.) immunizations with the tandem RBDs-Fc interacted with RBD proteins derived from SARS-CoV-2 variants. FIG. 11A. Ten g of tandem RBDs-Fc, or PBS in combination with 10 μg of CpG was i.n. administered into the age-matched 6-8 months-old C57B6/C mice. Mice were boosted twice at a 2-week interval following primary immunization. Bleeding was performed at the indicated time points. FIG. 11B. Anti-RBD-specific IgG antibody titers in the mouse sera. The RBD-specific antibody titers were measured by coating with RBD proteins from different SARS-CoV-2 variants in ELISA 14 days after the second boost. Tandem RBDs or MERS RBD were used as a positive or negative control, respectively. The IgG titers were measured in five representative mouse sera.

FIG. 12A-F. Intranasal immunization by the tandem RBDs-Fc induces protective immunity to intranasal (i.n.) challenge with ancestral SARS-CoV-2 virus. FIG. 12A. 10 μg of RBDs-Fc or PBS in combination with 10 μg of CpG was i.n. administered into 6-8-week-old human ACE-2 transgenic mice (n=15). Mice were boosted 14 and 28 days after primary immunization. Five to six mice in each group are euthanized at 5 dpi for sampling and virus titration. The remaining 10 mice in each group were subjected to the survival analysis. FIG. 12B. RBD-specific IgG Ab titers in sera of ACE2 mice. The RBD-specific IgG antibody titers were measured by coating the plates with RBDs protein in ELISA. FIG. 12C. Body-weight changes following the ancestral SARS-CoV-2 virus challenge. Seventeen days after the second boost, groups of 10 mice (RBDs-Fc group, n=10; PBS group, n=10) were i.n. challenged with the SARS-CoV-2 virus (2.5×10⁴ TCID₅₀) and weighed daily for 14 days. Mice were humanely euthanized at the end of the experiment or when a humane endpoint was reached. FIG. 12D. Survival following virus challenge. The percentage of human ACE2 transgenic mice protected after the i.n. challenge was shown by the Kaplan-Meier survival curve. FIG. 12E. Viral titers in the nasal turbinate, lung, and brain 5 days after the challenge. The virus titers in the samples of the immunized and PBS control mice (n=5) were determined. The presence of live virus was determined by CPE in Vero E6 cells cultured for 4 days. The viral titers were shown as TCID₅₀ from each animal sample. FIG. 12F. Histopathology of the lungs from the RBDs-Fc immunized or PBS control mice that were i.n. challenged. Lungs were collected 5 days post-challenge. The lung sections were stained with Hematoxylin-Eosin to determine the intensity of inflammation. The representative slides are shown. All scale bars represent 20 μm.

FIG. 13A-I. Intranasal immunization by the tandem RBDs-Fc induces protective immunity to intranasal (i.n.) challenge with the SARS-CoV-2 Delta variant. FIG. 13A. 10 μg of RBDs-Fc or PBS in combination with 10 μg of CpG was i.n. administered into 6-8-week-old human ACE-2 transgenic mice (n=15). Mice were boosted 14 and 28 days after primary immunization. Five to six mice in each group are euthanized at 5 dpi for sampling and virus titration. The remaining 10 mice in each group are subjected to the survival analysis. FIG. 13B, FIG. 13C. Serum anti-RBD-specific IgG antibody titers (FIG. 13B) and neutralizing antibodies (nAb, FIG. 13C) in hACE2 mice. The RBD-specific IgG antibody titers were measured by coating the plates with RBDs protein in ELISA; the nAb activity in the immunized sera was determined against Delta or Omicron A.1 strains by the micro-neutralization test, respectively. The data represent a geometric mean with 95% CI. FIG. 13D. Body-weight changes following the SARS-CoV-2 delta virus challenge. Seventeen days after the boost, groups of 10 mice (RBDs-Fc or PBS group, n=10) were i.n. challenged with the SARS-CoV-2 Delta virus (2.5×10⁴ TCID₅₀) and weighed daily for 14 days. Mice were humanely euthanized at the end of the experiment or when a humane endpoint was reached. FIG. 13E. Survival following virus challenge. The percentage of mice protected after the challenge was shown by the Kaplan-Meier survival curve. FIG. 13F. Viral titers in the nasal turbinate, lung, and brain 5 days after the challenge. The virus titers in the samples of the immunized and control mice (n=5) were determined. The presence of live virus was determined by CPE in Vero E6 cells cultured for 4 days. The viral titers were shown as TCID₅₀ from each animal sample. FIG. 13G. Histopathology of the lungs from the immunized mice that were i.n. challenged. Lungs were collected 5 days post-challenge. The lung sections were stained with Hematoxylin-Eosin (FIG. 13H, FIG. 13E) to determine the intensity of inflammation. The representative slides are shown. All scale bars represent 20 μm. FIG. 13H, FIG. 13I. Intranasal immunization of RBDs did not induce protective immune responses against viral infections, as determined by the body weight and survival following the challenge.

FIG. 14A-E. Sera from mice intranasally immunized by the tandem RBDs-Fc efficiently neutralize SARS-CoV-1, SARS-CoV-2, and MERS. 14A. Diagram demonstration of the fusion multiple RBDs from SARS-CoV-1, SARS-CoV-2, and MERS Spikes with human IgG Fc to create a pcDNA3 plasmid encoding a tandem RBDs-Fc fusion protein. Each RBD was bridged with a short Gly-Ser linker. A mutation was also made in the human IgG Fc fragment by replacing Lys 322 with Ala residues to remove the complement C1q binding site. 14B. Graphic demonstration of the putative expressed RBDs-Fc based on IgG Fc dimerization and RBD structure. The tandem RBDs-Fc fusion protein was purified by Protein A column from culture supernatants of the stable CHO cells that were transfected by the recombinant plasmid pcDNA3 encoding RBDs-Fc (14A). The purified tandem RBDs-Fc proteins were subjected to SDS-PAGE analysis and were visualized by Coomassie blue staining. 14C. Ten g of tandem RBDs-Fc, or PBS in combination with 10 μg of CpG was intranasally administered into the age-matched 6-8 months-old C57BL/6 mice. Mice were boosted twice at a 2-week interval following primary immunization. Bleeding was performed at the indicated time points. 14D. RBD-specific IgG antibody titers in mouse sera were measured by coating the plates with tandem RBDs protein in ELISA. 14E. inhibition of SARS-CoV-1, SARS-CoV-2, and MERS pseudoviruses by immunized mouse sera. SARS-CoV-1, SARS-CoV-2, and MERS pseudoviruses were used to infect HEK293T cells stably expressing human ACE2 or DPP4 in the presence of serially diluted mouse sera. NT₅₀ values were determined for sera from each mouse for neutralization titer that inhibits each pseudovirus entry by 50%. All the titers were measured in triplicates. The titers are presented as the mean plus the standard error of the mean (SEM).

FIG. 15A-B. Intramuscular immunization by the tandem RBDs induces RBD-specific IgG immune responses. FIG. 15A. Ten g of tandem RBDs, or PBS in combination with 10 μg of CpG was intramuscularly administered into the age-matched 6-8 months-old C57BL/6 mice. Mice were boosted twice at a 2-week interval following primary immunization. Bleeding was performed at the indicated time points. FIG. 15B. Anti-RBD-specific IgG Ab titers in the mouse sera. The RBD-specific Ab titers were measured by coating with RBD proteins in ELISA 14 days after the last boost. The IgG titers were measured in 5 representative mouse sera.

### DETAILED DESCRIPTION

The present disclosure relates to novel recombinant coronavirus proteins comprising tandemly arranged viral receptor binding domains (RBDs) fused to an Ig Fc domain (herein referred to as “RBDs-IgG Fc protein”). The different domains of the RBD-IgG Fc protein, i.e., the RBD and IgG Fc domains may be linked by one or more linker sequences. The terms “RBD” and “IgG Fc” refer to specific protein domains that are well-known by the person skilled in the art. The present disclosure also relates to novel recombinant coronavirus proteins comprising tandemly arranged viral receptor binding domains (RBDs) but lacking an IgG Fc domain (herein referred to as “RBDs protein”). While the disclosure below provides recombinant fusion proteins and their uses, it is understood that tandem RBDs may be linked to an FcRn targeting moiety using additional methods well known in the art such as, for example, chemical crosslinking, e.g., covalent bonding. Such a targeting moiety includes, for example, an IgG Fc domain. In a specific embodiment, the coronavirus is a SARS-CoV-2 virus. Such RBD-IgG Fc proteins, and RBDs proteins, are designed to mimic antigenic sites of the viral receptor binding domain for use as an effective and immunogenic vaccine.

As used herein, the term “coronavirus” is meant to include all microorganisms classified and identified as coronavirus. There are hundreds of coronaviruses, most of which circulate among such animals as pigs, camels, bats and cats. Coronaviruses are a large family of viruses that usually cause mild to moderate upper-respiratory tract illnesses, such as the common cold. However, coronaviruses have emerged from animal reservoirs over the past two decades to cause serious and widespread illness and death. Such coronaviruses include, for example, SARS coronavirus (SARS-CoV) causing severe acute respiratory syndrome (SARS), MERS coronavirus (MERS-CoV) causing Middle East respiratory syndrome (MERS) and SARS-CoV-2 causing coronavirus disease 2019 (COVID-19). While the disclosure below is directed to SARS-CoV-2 based fusion proteins, it is understood that said disclosure can be applied equally as well to other coronaviruses, variants, and their RBDs.

As used herein, the terms “protein”, “amino acid” and “polypeptide” are used interchangeably. The term “protein” refers to a sequence of amino acids composed of naturally occurring amino acids as well as derivatives thereof. The naturally occurring amino acids are well known in the art and are described in standard textbooks of biochemistry. Within the amino acid sequence, the amino acids are connected by peptide bonds. Further, the two ends of the amino acid sequence are referred to as the carboxyl terminus (C-terminus) and the amino terminus (N-terminus). The term “protein” encompasses essentially purified proteins or protein preparations and other proteins in addition. Further, the term also relates to protein fragments. Moreover, it includes chemically modified proteins. Such modifications may be artificial modifications or naturally occurring modifications such as phosphorylation, glycosylation, myristoylation, and the like.

The coronavirus spike glycoprotein binds to angiotensin-converting enzyme 2 (ACE2) via its receptor binding domain (RBD) thereby initiating the viral infection process. The RBDs, for use in the engineering of the disclosed fusion proteins, may be derived from a variety of different coronaviruses, including for example, SARS-CoV2, SARS-CoV1 and MERS-CoV as well as variants thereof. Such viruses and their corresponding RBDs are well known in the art and their sequences are publicly available, e.g., through Genebank. SARS-CoV2 variants include, but are not limited to, alpha, beta, delta, epsilon, kappa, gamma, lota, mu, theta and omicron variants. Such variants include, but are not limited to, BA.1, BA2.75, BA4-BA5, BAF.7, XBB.1 XBB1.5, BQ.1, BQ1.1 variants. RBD sequences to be included in the RBD-IgG Fc proteins, or RBDs proteins, disclosed herein include, for example, those RBDs located within SEQ ID NOs 2, 4, 6, 8, 10, 12, 14 or 16, a sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof. In an embodiment, RBDs derived from newly identified variants, may be used in the construction of RBDs-IgG Fc proteins, or RBDs proteins, as disclosed herein.

As disclosed in detail below, the provided RBDs-IgG Fc proteins, or RBDs proteins, disclosed herein may utilize different combinations and orientations of the tandemly arranged RBDs as well as, optionally, linker sequences linking RBDs, or fragments thereof. In an embodiment, for the RBDs-IgG Fc protein, the tandem arranged RBD domains are linked to an IgG Fc domain. In a specific embodiment, the RBDs-IgG Fc proteins, or RBDs proteins, comprise 2 or more tandemly arranged RBDs. In another embodiment, the proteins comprise 2-12 tandemly arranged RBDs.

In a specific aspect, the RBDs-IgG Fc protein comprises an IgG Fc domain, including those represented from the different IgG subclasses. In an embodiment, the IgG domain is an IgG1 subclass. In a specific embodiment, the IgG Fc domain is a human IgG Fc domain. In an embodiment, the Lys322 residue is replaced with Ala (K322A). In a specific embodiment, the IgG Fc domain comprises the amino acid sequence of SEQ ID NO: 18, a sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof.

In another embodiment, the RBDs-IgG Fc proteins, or RBDs proteins comprise the amino acid sequences of SEQ ID NOs: 2, 4, 6, 8, 10, 12, 14 and 16, a sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or fragments thereof.

Included are RBDs-IgG Fc proteins, or RBDs proteins, that include RBDs as disclosed above, but which contain amino acid substitutions or deletions and which are nevertheless able to elicit a protective immune response when included in a vaccine composition.

Each of the protein domains of the RBDs-IgG Fc protein, or RBDs protein, e.g., the RBD and, optionally, the IgG Fc domain, may be linked by one or more amino acid residues. The term “linker” refers to a short, non-native peptide sequence that links two proteins or fragments of a protein. Such linker sequences include any linker sequence that permits the folding of the different protein domains to mimic as closely as possible the naturally occurring domains. In an aspect, the linker sequence is a polypeptide having 1-70 amino acids. The linker sequence may be a polypeptide of 2-50 amino acids. The linker may have a length of 1, 2, 3, 4, 5, 6, 7, 8, 9, 10, 11, 12, 13, 14, 15, 16, 17, 18, 19, 20, 21 or 22 amino acids. In an embodiment the linker sequence may be a Gly-Ser amino acid linker. In an embodiment, the linker may comprise multiple Gly-Ser amino acid residues. Further, a signal sequence may be included in the RBD-IgG Fc protein or RBDs protein.

The RBDs-IgG Fc proteins disclosed herein can utilize different combinations and orientations of the tandem RBD and IgG Fc domains. Similarly, the RBDs proteins disclosed herein can utilize different combinations and orientations of the tandemly arranged RBD.

In addition to the recombinantly expressed fusion proteins, e.g., IgG Fc proteins or RBDs proteins, disclosed herein, it is understood that tandem RBDs may also be linked to an IgG Fc domain using additional methods well known in the art. Such linkage may be accomplished, for example, through a chemical reaction resulting in crosslinking of the tandem RBDS to an IgG Fc domain. In some instances, the crosslinking may be accomplished using peptide linkers.

In an embodiment, tandemly arranged RBDs may be targeted to mucosal tissue through linkage to a targeting moiety having an affinity for such mucosal tissue. In an embodiment, the mucosal targeting moiety is a FcRn targeting moiety. In an embodiment, tandemly arranged RBDs may be targeted to a FcRn through linkage to a FcRn targeting moiety other than an IgG Fc domain. Such a FcRn targeting moiety is one that targets binding of the tandemly arranged RBDs to the FcRn. Such targeting moieties may comprise a protein, polypeptide, or chemical entity having a binding affinity for the FcRN. A FcRn targeting moiety may be, for example, an antibody binding domain that recognizes and binds to the FcRN. Such linkage may be accomplished through recombinant expression of fusion proteins comprising tandem RBDs fused to a mucosal or FcRN targeting moiety. Alternatively, linkage of the tandem RBDs to the mucosal or FcRn targeting moiety may be accomplished through chemical crosslinking, e.g., covalently bonding, of the mucosal or FcRn targeting moiety to the tandem RBDs. In some instances, the crosslinking may be accomplished using peptide linkers.

The present disclosure also relates to nucleic acid molecules encoding for the RBDs-IgG Fc proteins, or the RBDs proteins, disclosed above. “Nucleic acid” or “nucleic acid sequence” or “nucleotide sequence” refers to polynucleotides including DNA molecules, RNA molecules, cDNA molecules or derivatives. The term encompasses single as well as double stranded polynucleotides. In a specific embodiment, the nucleic acid includes a cDNA or mRNA molecule capable of encoding the RBD-IgG Fc proteins, or the RBDs proteins, disclosed herein.

The nucleic acids of the present disclosure encompass isolated polynucleotides (i.e., isolated from its natural context) and genetically modified forms. Moreover, included are chemically modified polynucleotides including naturally occurring modified polynucleotides such as glycosylated or methylated polynucleotides or artificially modified ones such as biotinylated polynucleotides. The terms “nucleic acid” and “polynucleotide” also specifically include nucleic acids composed of bases other than the five biologically occurring bases (adenine, guanine, thymine, cytosine and uracil).

The term “identity” or “sequence identity” is known in the art and refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference).

The protein sequences, or nucleic acid sequences, disclosed herein can further be used as a “query sequence” to perform a search against public databases to, for example, to identify other coronavirus family members and their corresponding RBDs, or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to proteins of the disclosure. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

Methods of preparing a recombinant RBDs-IgG Fc protein, or a RBDs protein, are provided. The preparation method may be performed through recombinant DNA technology known in the art using a nucleic acid encoding the RBDs-IgG Fc protein, or the RBDs protein. This method includes (i) preparing an expression vector having a nucleic acid encoding the RBDs-IgG Fc protein or the RBDs protein, (ii) transforming the expression vector into host cells, (iii) culturing the transformed host cells, and optionally, (iv) isolating and purifying the RBDs-IgG Fc protein, or the RBDs protein, from the resultant culture broth.

The RBDs-IgG Fc protein, or the RBDs protein, may also be chemically synthesized based on the RBDs-IgG Fc protein, or the RBDs protein, protein amino acid sequence. Such chemical synthesis methods are well known in the art, and, for example, solid-phase synthesis technology, solution-phase synthesis technology and the like may be used, and commercially available automated DNA synthesizers and the like using these technologies may be used. (See, Nucl. Acid Res. 14:5399-5467, 1986; Tet. Lett. 27:5575-5578, 1986; Nucl. Acid Res. 4:2557, 1977; and Lett., 28:2449, 1978) and the like.

When the preparation method is through recombinant DNA technology, the expression vector may be a nucleic acid in the form of a plasmid, a cosmid, a phagemid, a phage, a viral vector, or the like. Depending on the host microorganism, an appropriate vector may be purchased among commercially available vectors or may be used after being purchased and modified.

For expression vector construction including recombinant DNA technology, reference may be made to Sambrook et al., Molecular Cloning, A Laboratory Manual, Cold Spring Harbor Laboratory Press, (2001), F M Ausubel et al, Current Protocols in Molecular Biology, John Wiley amp; Sons, Inc. (1994), and Marston, F (1987) DNA Cloning Techniques) and the like.

The expression vector may include regulatory sequences that affect transcription and translation of the RBDs-IgG Fc, or the RBDs, encoding nucleic acid by being operably linked to the nucleic acid, in addition to the nucleic acid encoding the RBDs-IgG Fc protein, or the RBDs protein. Such a regulatory sequence usually includes a promoter sequence, a transcription termination signal sequence (polyadenylation signal), and the like. As used herein, the term “being operably linked” means a linkage such that the transcription and/or translation of a nucleic acid are affected. For example, if a promoter affects the transcription of a nucleic acid linked thereto, the promoter and the gene are regarded as operably linked. Regulatory sequences also include enhancer sequences that function to regulate the transcription of a nucleic acid.

As used herein, the term “promoter” refers to a nucleic acid sequence having a function of controlling transcription of one or more nucleic acids, which is located upstream (5′ side) of the transcription initiation point of a nucleic acid and includes a binding site for a DNA-dependent RNA polymerase, a transcription initiation point, a transcription factor binding site, and the like. So long as the promoter is capable of expressing the target nucleic acid linked thereto, any of a constitutive promoter (a promoter that induces expression constantly in a certain organism) and an inducible promoter (a promoter that induces expression of a target gene in response to a certain external stimulus) may be used. In an embodiment, a promoter suitable for a certain host microorganism is used. Enhancer sequences may also be employed to control the expression of the RBDs-IgG Fc, or RBDs, encoding nucleic acids.

The expression vector may be configured to include a terminator sequence which is a transcription termination sequence, in addition to the promoter. The terminator sequence is a sequence that acts as a poly(A) addition signal (polyadenylation signal) to increase the completeness and efficiency of transcription. Suitable terminator sequences, depending on the host microorganism, are known in the art.

The expression vector may further include a selectable marker gene. The selectable marker gene is a gene encoding a trait that enables the selection of a host microorganism containing such a marker gene and is generally an antibiotic resistance gene.

The expression vector may also include a restriction enzyme recognition site for easy cloning of the RBD-IgG Fc, or the RBDs, encoding nucleic acid. The expression vector may then be transformed into a host microorganism for expression of the proteins.

In a specific embodiment, RBDs-IgG Fc, or RBDs, encoding nucleic acid may be introduced into recombinant delivery vectors such as genetically engineered viral or bacterial vectors. Viral vectors include bacteriophages, herpesvirus, adenovirus, poliovirus, vaccinia virus, defective retroviruses, adeno-associated virus (AAV), lentiviruses, plant viruses, and hybrid vectors. Methods of transforming viral vectors with a recombinant DNA construct are also well described in the art.

The present disclosure provides recombinant cells into which expression vectors designed for the expression of RBDs-IgG Fc proteins, or RBDs proteins, have been introduced. Such cells include bacteria as well as eukaryotic cells, such as CHO cells. Transformation refers to the modification of a genotype of a cell due to the introduction of a nucleic acid, and the introduced nucleic acid may be present independently of the genome of the host cell or in the state of being incorporated into the genome of the host cell.

Methods of transforming the expression vector into the host cell are also known in the art, and any of the known methods may be selected and used. For example, when the host cell is prokaryotic cells such as Escherichia coli, the transformation may be carried out through a CaCl₂ method, a Hanahan method, an electroporation method, a calcium phosphate precipitation method, or the like, and when the host cell is eukaryotic cells such as yeast or mammalian cells, a microinjection method, a calcium phosphate precipitation method, an electroporation method, a liposome-mediated transfection method, a DEAE-dextran treatment method, a gene bombardment method, or the like may be used. Regarding details of the transformation method, reference may be made to (Cohen, S. N. et al., Proc. Natl. Acad. Sci. USA, 9:2110-2114 (1973); Hanahan, D., J. Mol. Biol., 166:557-580 (1983); Dower, W. J. et al., Nucleic. Acids Res., 16:6127-6145 (1988); Capecchi, M. R., Cell, 22:479 (19800; Graham, F. L. et al., Virology, 52:456 (1973); Neumann, E. et al., EMBO J., 1:841 (1982); Wong, T. K. et al., Gene, 10:87 (1980); Gopal, Mol. Cell Biol., 5:1188-1190 (1985); Yang et al., Proc. Natl. Acad. Sci., 87:9568-9572 (1990); Maniatis et al., Molecular Cloning: A Laboratory Manual, Cold Spring Harbor Laboratory (1982); Hitzeman et al., J. Biol. Chem., 255, 12073-12080 (1980); and Luchansky et al Mol. Microbiol. 2, 637-646 (1988), etc.)

The host cell that may be used for transformation in the method of the present disclosure may be prokaryotic or eukaryotic cells. As the prokaryotic cells, any gram-positive bacteria and gram-negative bacteria may be used. In a specific embodiment, Escherichia coli is used. In order to optimize expression and maintain the functions of the RBDs-IgG Fc protein, or the RBDs protein, in Escherichia coli, the cell may have impaired protease activity. Also, the nucleic acid sequence encoding the RBD-IgG Fc protein, or the RBDs protein, may be optimized with a codon usage preferred in the host to which the protein is to be expressed. (see, Wada et al., Nucleic Acids Res. 20:2111-2118 (1992)).

The host cell transformed above is cultured, thus producing the recombinant RBD-IgG Fc protein, or the recombinant RBDs protein. The culture of the transformed host cell may be performed through any method known in the art. As the medium used for cell culture, any of a natural medium and a synthetic medium may be used, so long as it contains a carbon source, a nitrogen source, a trace element, etc. which may be efficiently used by the transformed host cell. When animal cells are used as host cells, Eagle's MEM (Eagle's minimum essential medium, Eagle, H. Science 130:432 (1959)0, α-MEM (Stanner, C. P. et al., Nat. New Biol. 230:52 (1971)), Iscove's MEM (Iscove, N. et al., J. Exp. Med. 147:923 (1978)), DMEM (Dulbecco's modification of Eagle's medium, Dulbecco, R. et al., Virology 8:396 (1959)) or the like may be used. Regarding details of the medium, see, for example, R. Ian Freshney, Culture of Animal Cells, A Manual of Basic Technique, Alan R. Liss, Inc., New York.

Methods of isolating and purifying the RBDs-IgG Fc protein, or the RBDs protein, are also well known in the art, and any known method may be used. Examples thereof may include ultrafiltration, gel filtration, ion exchange chromatography, affinity chromatography (when labeled peptides are bound), HPLC, hydrophobic chromatography, isoelectric point chromatography, and combinations thereof. In a specific embodiment, the RBDs-IgG Fc protein, or the RBDs protein, may be engineered to include a HIS-tag as a means for affinity chromatography.

Also disclosed is a nanoparticle comprising a RBDs-IgG Fc protein or RBDs protein. Such nanoparticles can be natural or synthetic and may be incorporated into a vaccine composition. They can be created from biological molecules or from non-biological molecules. In some cases, the protein complex is crosslinked to a polymer or lipid on the nanoparticle surface. In embodiments, the protein complex is adsorbed onto the nanoparticle surface. In some embodiments, the protein complex is adsorbed onto the nanoparticle surface and then crosslinked to the nanoparticle surface. In some embodiments, the protein complex is encapsulated into the nanoparticle.

In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof.

In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero-3-phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phos phatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000](DPPE-PEG2000), 1,2-distearoyl-sn-glycero-3-phosphoethanolamine-N-[methoxy(polyethylene glycol)-1000](DSPE-PEG2000), and cholesterol.

In some embodiments, the RBDs-IgG Fc protein is coated on the nanoparticle using a crosslinking agent. In some embodiments, the RBDs-IgG Fc protein is adsorbed onto the nanoparticle surface. In some embodiments, the RBDs-IgG Fc protein is adsorbed onto the nanoparticle surface followed by covalent crosslinking of the RBDs-IgG Fc protein to the nanoparticle surface using a crosslinking agent.

In some embodiments, the RBDs protein is coated on the nanoparticle using a crosslinking agent. In some embodiments, the RBDs protein is adsorbed onto the nanoparticle surface. In some embodiments, the RBDs protein is adsorbed onto the nanoparticle surface followed by covalent crosslinking of the RBDs protein to the nanoparticle surface using a crosslinking agent.

Crosslinking agents suitable for crosslinking the proteins to produce the nanoparticle, or to coat the proteins on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH, BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

The present disclosure provides a vaccine composition containing a RBDs-IgG Fc protein, or a RBDs-IgG Fc protein encoding nucleic acid, as an active ingredient. The present disclosure further provides a vaccine composition containing a RBDs protein, or a RBDs protein encoding nucleic acid, as an active ingredient. As used herein, the term “vaccine” refers to a composition able to prevent or reduce the infection or re-infection with a coronavirus, reducing the severity of symptoms or eliminating symptoms of coronavirus infection, or substantially or completely removing the disease caused by the coronavirus, by inducing an immune response to the coronavirus in a host. In an embodiment, the term “vaccine” refers to a composition able to prevent or reduce the infection or re-infection with SARS-CoV-2, reducing the severity of symptoms or eliminating symptoms of COVID-19, or substantially or completely removing SARS-CoV-2 or the disease by SARS-CoV-2, by inducing an immune response to SARS-CoV-2 in a human host. Thus, the vaccine composition disclosed herein may be administered prophylactically to a subject, i.e., a human, before infection with SARS-CoV-2, or may be therapeutically administered to subjects after infection with SARS-CoV-2. Here, the term “immune response” includes either or both of a humoral immune response and a cellular immune response.

Also provided is the in vivo administration of a nucleic acid encoding the RBD-IgG Fc protein, or the RBDs protein, so that the protein is expressed in the immunized subject (e.g., nucleic acid vaccine, DNA or RNA vaccine). In an embodiment, the nucleic acid includes a nucleotide sequence that encodes the protein operably linked to regulatory elements needed for gene expression, such as a promoter, an initiation codon, a stop codon, enhancer, and a polyadenylation signal. Regulatory elements are typically selected that are operable in the species to which they are to be administered.

The nucleic acid of the vaccine composition can be “naked” DNA, cDNA or mRNA or can be operably incorporated in a vector. Nucleic acids may be delivered to cells in vivo using methods well known in the art such as direct infection of DNA, receptor-mediated DNA uptake, viral-mediated transfection or non-viral transfection and lipid-based transfection, all of which may involve the use of vectors. Naked DNA may also be introduced into cells by complexing the DNA to a cation, such as polylysine, which is coupled to a ligand for a cell-surface receptor (see for example Wu, G and Wu, C. H. (1988) J. Biol. Chem. 263: 14621; Wilson et al. (1992) J. Biol. Chem. 267: 963-967, and U.S. Pat. No. 5,166,320). Binding of the DNA ligand complex to the receptor may facilitate the uptake of the DNA by receptor-mediated endocytosis. A DNA ligand complex linked to adenovirus capsids which disrupt endosomes, thereby releasing material into the cytoplasm, may be used to avoid degradation of the complex by intracellular lysosomes (see for example Curiel et al. (1991) Proc. Natl. Acad. Sci. USA 88: 8850; Cristriano et al. (1993) Proc. Natl. Acad. Sci. USA 90: 2122-2126).

Useful delivery vectors for inclusion in the vaccine compositions include biodegradable microcapsules, immuno-stimulating complexes (ISCOMs) or liposomes, and genetically engineered attenuated live vectors such as viruses or bacteria. Viral vectors include bacteriophages, herpes virus, adenovirus, polio virus, vaccinia virus, defective retroviruses and adeno-associated virus (AAV). Methods of transforming viral vectors with an exogenous DNA construct are also well described in the art.

Liposome vectors may also be used for delivery of nucleic acids or proteins. Such liposome vectors may be unilamellar or multilamellar vesicles, having a membrane portion formed of lipophilic material and an interior aqueous portion. The aqueous portion is used to contain the polynucleotide material to be delivered to the target cell. In general, the liposome forming materials have a cationic group, such as a quaternary ammonium group, and one or more lipophilic groups, such as saturated or unsaturated alkyl groups having about 6 to about 30 carbon atoms. One group of suitable materials is described in European Patent Publication No. 0187702, and further discussed in U.S. Pat. No. 6,228,844 to Wolff et al., the pertinent disclosures of which are incorporated by reference. Many other suitable liposome-forming cationic lipid compounds are described in the literature. See, e.g., L. Stamatatos, et al., Biochemistry 27:3917 3925 (1988); and H. Eibl, et al., Biophysical Chemistry 10:261 271 (1979). Alternatively, a microsphere such as a polylactide-co-glycolide biodegradable microsphere can be utilized. A nucleic acid construct, or protein, is encapsulated or otherwise complexed with the liposome or microsphere for delivery of the nucleic acid to a tissue, as is known in the art.

Alternatively, the nucleic acid (e.g., DNA or mRNA) may be incorporated in a cell in vitro or ex vivo by transfection or transformation and the transfected or transformed cell (e.g., an immune cell such as a dendritic cell), which expresses the RBDs-IgG Fc protein (or a fragment thereof), or which expresses the RBDs protein (or a fragment thereof), may be administered to the host. Following administration, the cell will express the RBDs-IgG Fc protein (or a fragment thereof), or the RBDs-IgG Fc protein (or a fragment thereof), in the host which will in turn lead to the induction of an immune response directed against the RBDs-IgG Fc, or RBDs, protein, polypeptide or fragment thereof.

The vaccine compositions provided herein may be prepared in any suitable and pharmaceutically acceptable formulation. It may be provided in the form of an immediately administrable solution or suspension, or a concentrated crude solution suitable for dilution before administration or may be provided in a form capable of being reconstituted, such as a lyophilized, freeze-dried, or frozen formulation.

The vaccine composition may contain a pharmaceutically acceptable carrier in order to be formulated. The carrier typically includes a diluent, an excipient, a stabilizer, a preservative, and the like. Suitable examples of the diluent may include non-aqueous solvents such as propylene glycol, polyethylene glycol, vegetable oil such as olive oil and peanut oil, or aqueous solvents such as saline (for example, 0.8% saline), water (for example, 0.05 M phosphate buffer) containing a buffer medium, and the like, suitable examples of the excipient may include starch, glucose, lactose, sucrose, gelatin, malt, rice, flour, chalk, silica gel, sodium stearate, glycerol monostearate, talc, sodium chloride, anhydrous skimmed milk, glycerol, propylene, glycol, water, ethanol and the like, and suitable examples of the stabilizer may include carbohydrates such as sorbitol, mannitol, starch, sucrose, dextran, glutamate, and glucose, or proteins such as animal, vegetable or microbial proteins such as milk powder, serum albumin and casein. Suitable examples of the preservative may include thimerosal, merthiolate, gentamicin, neomycin, nystatin, amphotericin B, tetracycline, penicillin, streptomycin, polymyxin B and the like.

The vaccine composition of the present disclosure may further contain an adjuvant. The adjuvant may be composed of one or more substances that enhance the immune response to an antigen, i.e., the RBDs-IgG Fc protein or the RBDs protein. The adjuvant may function as a tissue reservoir that slowly releases an antigen and/or as a lymphoid system activator that nonspecifically enhances an immune response (Hood et al., Immunology, Second Ed., 1984, Benjamin/Cummings: Menlo Park, Calif., p. 384). Examples of the antigen adjuvant may include complete Freund, incomplete Freund, saponin, gel-type aluminum adjuvants, surface active substances (e.g. lysolecithin, pluronic polyols, polyanions, peptides, oils or hydrocarbon emulsions), vegetable oil (cottonseed oil, peanut oil, corn oil, sunflower oil, etc.), vitamin E acetate and the like. The adjuvant may consist of monophosphoryl lipid A (MPL) from Salmonella Minnesota or QS-21, a purified active fraction of the bark of Chilean tree Quillaja saponaria.

Among adjuvants applicable to the human body, an aluminum adjuvant is most widely used, and examples of the aluminum adjuvant may include gel-type aluminum salts such as aluminum phosphate, potassium aluminum sulfate, aluminum hydroxide and the like. The aluminum adjuvant is generally known to elicit a Th2-type immune response and enhance vaccine efficacy (Sokolovska A et al., Vaccine. 2007 Jun. 6; 25(23):4575-85; O'Hagan D T and Rappuoli R., Pharm Res. 2004 September; 21(9):1519-30.). Methods of preparing the aluminum adjuvant are known in the art (R. Bomford. Immunological Adjuvants and Vaccines. NATO ASI Series 1989; 179: 35-41; Vogel F R AND Powell M F. Pharm. Biotechnol. 1995; 6: 141-228; Derek T. O'Hagan, Methods in Molecular Medicine. 2000 Apr. 15; 42: 65-90), and the aluminum adjuvant may be used through direct preparation or by purchasing a commercially available product. Examples of commercially available product thereof may include Aluminum hydroxide Gel products (Sigma) and Alhydrogel products (BRENNTAG), in addition to the 2% Alhydrogel (InvivoGen).

The provided vaccine composition may be produced in an arbitrary unit dose. A unit dose refers to the amount of the active ingredient and the pharmaceutically acceptable carrier contained in each product packaged for use in one or more administrations to a subject, such as a human, and an appropriate amount of such active ingredient and carrier is an amount that may function as a vaccine when inoculation with the vaccine composition of the present disclosure is performed one or more times, and such an amount may be determined non-clinically or clinically as understood by those skilled in the art.

A method of vaccinating a subject for coronavirus is provided that includes administering the disclosed coronavirus vaccine composition to a subject in need thereof. A method of vaccinating a subject for SARS-CoV-2 is provided that includes administering the disclosed SARS-CoV-2 vaccine composition to a subject in need thereof. Said subjects include any animal that serves as a host for a coronavirus. Said subject may be an animal under the care of a veterinarian. Said subject may be a mammal. Said subject may be a human.

The disclosed vaccine compositions may be administered in a number of ways. For example, the disclosed vaccine composition can be administered orally, intravenously, subcutaneously, transdermally (e.g., by microneedle), intraperitoneally, ophthalmically, vaginally, rectally, sublingually, or by inhalation. The vaccine composition of the present disclosure may be administered in a controlled release system including, for example, a liposome, a transplantation osmotic pump, a transdermal patch, and the like.

Methods of systemic delivery include those methods known in the art that provide delivery of the active molecule (e.g. the RBDs-IgG Fc protein or RBDs protein) to the circulatory system with distribution throughout the body. Systemic delivery methods include intramuscular, intravenous, subcutaneous, intraperitoneal, and oral. As will be understood, any method of systemic delivery is suitable for use as a means for vaccination. Particularly suitable methods of systemic delivery include intramuscular and intravenous delivery.

In a specific embodiment, the vaccine compositions are formulated for intranasal administration. Intranasal administration of the vaccine composition, if used, is generally characterized by inhalation. Compositions for nasal administration can be prepared so that, for example, the RBDs-IgG Fc protein can be administered directly to the mucosa (e.g., nasal and/or pulmonary mucosa).

Optionally, such intranasal vaccine compositions may further advantageously comprise a mucoadhesive, such as cellulose derivatives, polyacrylates, a starch, chitosan, glycosaminoglycans, hyaluronic acid, and any combination thereof. The mucoadhesive may be present in the composition at about 0.1% to about 10% by weight. For example, the vaccine can be formulated for intranasal delivery as a dry powder, as an aqueous solution, an aqueous suspension, a colloidal suspension, a water-in-oil emulsion, a micellar formulation, or as a liposomal formulation.

Methods for mucosal delivery include those methods known in the art that provide delivery of the composition to mucous membranes. Mucosal delivery methods include intranasal, intrabuccal, and oral. In some embodiments, the administration is intranasal.

In these embodiments, the RBDs-IgG Fc vaccine composition may be formulated to be delivered to the nasal passages or nasal vestibule of the subject as droplets, an aerosol, micelles, lipid or liquid nanospheres, liposomes, lipid or liquid microspheres, a solution spray, or a powder. The composition can be administered by direct application to the nasal passages, or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule. In some embodiments, the compositions of present invention can be delivered using a nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediate prior to use and squeezed or squirted into the nasal passages.

The dose of the vaccine composition may be determined by a medical practitioner in consideration of patient characteristics such as age, weight, gender, symptoms, complications, and the incidence of other diseases. Further, the temporal interval of administration and the number of administrations may be determined in consideration of the dosage form that is used, the half-life of the active ingredient in the blood, and the like.

The exact amount of the vaccine composition required may vary from subject to subject, depending on the species, age, weight and general condition of the subject and its mode of administration and the like. Thus, it is not possible to specify an exact amount for every composition. However, an appropriate amount can be determined by one of skill in the art using only routine experimentation given the teachings herein. For example, effective dosages and schedules for administering the vaccine compositions may be determined empirically, and making such determinations is within the skill in the art. The dosage ranges for the administration of the vaccine compositions are those large enough to produce the desired effect in which the symptoms of the disorder are affected. The dosage should not be so large as to cause adverse side effects, such as unwanted cross-reactions, anaphylactic reactions, and the like. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The terms “treat/treating/treatment” and “prevent/preventing/prevention” as used herein, refers to eliciting the desired biological response, i.e., a therapeutic and prophylactic effect, respectively. In accordance with the present disclosure, the therapeutic effect includes one or more of a decrease/reduction in the severity of the disease (e.g., a reduction or inhibition of infection), a decrease/reduction in symptoms and disease related effects, an amelioration of symptoms and disease-related effects, and an increased survival time of the affected host, following administration of the vaccine composition. A prophylactic effect may include a complete or partial avoidance/inhibition or a delay of infection, and an increased survival time of the affected host, following administration of the vaccine composition.

Also encompassed by the methods, uses, pharmaceutical compositions and kits of the present disclosure is passive immunization, which is the injection of antibodies or antiserum, previously generated against a RBD-IgG Fc protein, or a RBDs protein, in order to protect or cure a recipient host of an infection or future infection. Protection fades over the course of a few weeks during which time the active immunization with protein and/or DNA (as described above) will have time to generate a lasting protective response. Serum for passive immunization can be generated by immunization of donor animals using the RBD-IgG Fc protein, or RBDs protein. This serum, which contains antibodies against the antigens, can be used immediately or stored under appropriate conditions. It can be used to combat coronavirus infections, e.g., COVID-19 infections or as a prophylactic (Tuchscherr et al., 2008).

Toxicity or efficacy of vaccine components to elicit an immune response can be determined by standard procedures in cell cultures or experimental animals. Data obtained from cell culture assays and laboratory animal studies can be used in formulating a range of dosage for use in humans. The dosage of such components lies, for example, within a range of administered concentrations that include efficacy with little or no toxicity. The dosage may vary within this range depending upon the dosage form employed and the route of administration utilized.

The vaccine compositions may, if desired, be presented in a pack or dispenser device which may contain one or more-unit dosage forms containing the RBDs-IgG Fc, or RBDs, protein. The pack may for example include metal or plastic foil, such as a blister pack. The pack or dispenser device may be accompanied by instructions for administration to subjects, especially humans. Associated with such container(s) can be a notice in the form prescribed by a governmental agency regulating the manufacture, use or sale of pharmaceuticals or biological products, which notice reflects approval by the agency of manufacture, use or sale for human administration. Thus, a kit is provided that includes the RBDs-IgG Fc protein, and/or the RBDs protein, as described herein. In one specific aspect the kit further includes instructions for the treatment and/or prophylaxis of COVID-19.

Unless otherwise defined, all technical and scientific terms used herein have the same meaning as commonly understood by one skilled in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example 1

Epithelial monolayers lining the airway polarize into the apical and basolateral plasma membrane domains, which are separated by intercellular tight junctions. The neonatal Fc receptor (FcRn) is expressed in these epithelial monolayers and mediates the transfer of IgG antibodies across the polarized epithelium. By transferring IgG, FcRn provides a line of humoral defense at mucosal surfaces, in addition to transferring maternal immunity to neonates. A hallmark of FcRn is its interaction with IgG antibody in a pH-dependent manner, binding IgG at acidic pH (6.0-6.5) and releasing IgG at neutral or higher pH. FcRn primarily resides within low pH endosomes and binds IgG through the Fc region. Normally, IgG enters epithelial cells via pinocytotic vesicles that fuse with acidic endosomes. IgG bound to FcRn then enters a non-degradative vesicular transport pathway within epithelial cells. Bound IgG is transported to the apical or basolateral surface and released into the lumen or submucosa upon physiological pH. Evidence of IgG transport across the respiratory epithelia by FcRn indicates that FcRn might also transport a vaccine antigen, if fused with the Fc portion of IgG, across the respiratory mucosal barrier.

The receptor-binding domain (RBD) of the coronavirus spike is the primary target of the neutralizing antibodies elicited by natural infection or vaccination. The major antibody-neutralizing epitopes in the RBD account for more than 90% of all neutralizing activity against SARS-CoV-2. It was tested whether an antigen comprising and/or consisting of multiple RBDs derived from various SARS-CoV-2 variants can provide an effective mucosal vaccine against major circulating variants. As described below, a tandem RBDs was produced from SARS-CoV-2 variants of concerns or interests and the capacity of FcRn to nasally deliver this tandem RBDs antigen across the airway epithelial barrier and induce broad mucosal and systemic immunity was studied. Protective immune responses and mechanisms relevant to this nasal immunization were tested in wild-type and human angiotensin-converting enzyme 2 (ACE2) transgenic mice. The data indicates that FcRn-mediated nasal delivery of tandem RBDs antigen induces high levels of long-lasting antibody immune responses and provides broad protection against infections by SARS-CoV-2 and its variants. The data further indicates that FcRn-targeted nasal delivery of multiple RBDs of the SARS-CoV-2 spike protein comprises an effective mucosal vaccination strategy against the SARS-CoV-2 and its variants of concerns. Because almost all coronaviruses use their spike RBDs to engage with the cognate cellular receptors for their entry into cells, the nasal vaccination strategy using FcRn-targeted tandem RBDs antigens derived from different coronaviruses, such as betacoronavirus, can also be used to develop a pan-coronavirus vaccine against the emerging coronaviruses and their variants.

Construction and Expression of the Tandem RBDs Derived from SARS-CoV-2 and its Variants.

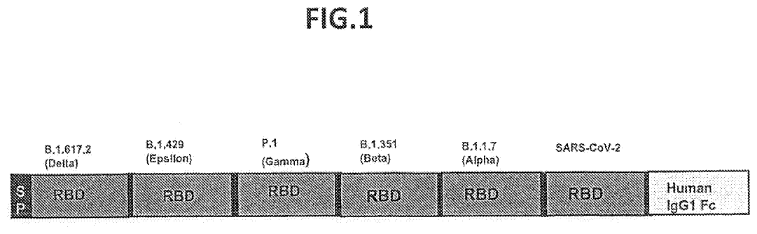

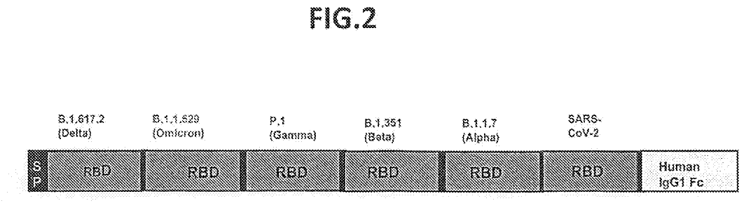

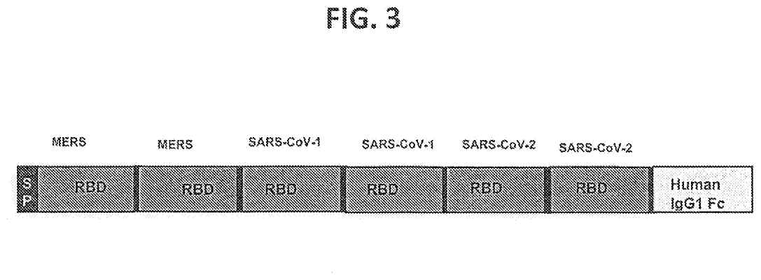

Several SARS-CoV-2 variants of concern or interest have emerged; as such, it would be beneficial to produce a nasal vaccine that protects against these variants. Accordingly, a tandem protein was produced containing six RBDs of SARS-CoV-2 and its variants (B.1.1.7, B.1.351, B.1.427, P.1., and B.1.617.2) (FIG. 1). Subsequently, because of the emergence of the Omicron variant, another tandem RBDs protein was produced containing six RBDs of SARS-CoV-2 and its variants (B.1.1.7, B.1.351, B.1.1.529, P.1., and B.1.617.2) (FIG. 2). Similarly, a plasmid was produced expressing a tandem RBDs of SARS-CoV-1, SARS-CoV-2, and MERS (FIG. 3). To target FcRn, the human IgG1 isotype that has the highest affinity for activating FcγRI, but the lowest affinity for inhibitory FcγRIIB was used. In human IgG1 Fc, the complement C1q-binding motif was eliminated (K322A) (FIG. 1-3), allowing one to produce a non-lytic vaccine antigen. The IgG Fc was fused in frame with the tandem RBDs (FIG. 1-3), generating plasmids pcDNA3RBDs-Fc that express a tandem RBDs from at least six different coronaviruses.

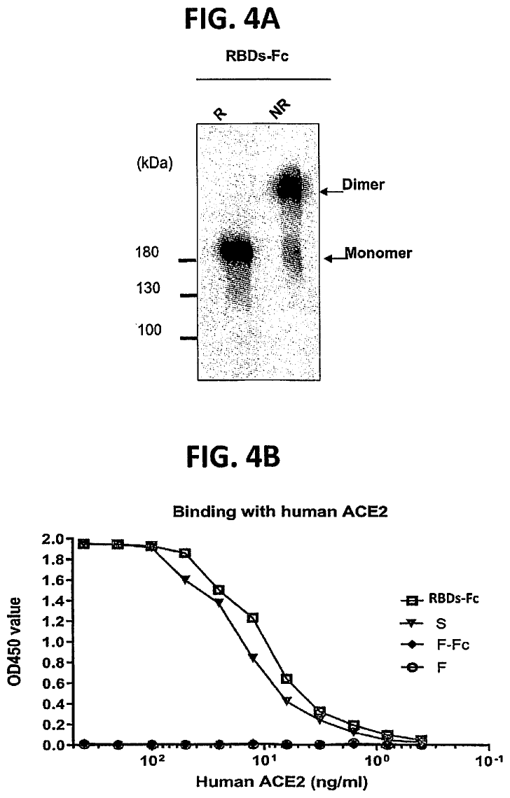

After transfection of CHO cells, a tandem protein comprising and/or consisting of six RBDs of SARS-CoV-2 and variants was successfully produced (FIG. 4A). In a coupling denaturing SDS-PAGE-Western blotting assay, it was found that the secreted proteins exhibited monomers under reducing (R) condition, but displayed dimers under non-reducing (NR) conditions (FIG. 4A), suggesting the secreted tandem RBDs-Fc proteins form an IgG-like structure. The dimerization of the RBDs-Fc indicates that at least 12 RBDs from different coronaviruses.

To determine whether the tandem RBDs-Fc proteins bind to FcRn, the ability of RBDs-Fc proteins to interact with staphylococcal Protein A was assessed, as the IgG Fc binding sites for both FcRn and Protein A overlap. The tandem RBDs-Fc proteins interacted with Protein A, indicating that the tandem RBDs-Fc maintains the structural integrity required to interact with FcRn.

It was further determined if the tandem RBDs portion of the RBDs-Fc protein maintains its conformation for binding to the ACE2. It was observed that the tandem RBDs-Fc secreted from CHO stable cell lines interacted with human ACE2 in an ELISA assay (FIG. 4B). The capacity of the tandem RBDs-Fc in binding to human ACE2 was similar to that of the trimeric spike proteins from SARS-CoV-2. To show the binding specificity, as a negative control, the trimeric F-Fc or F protein from human respiratory syncytial virus (RSV) was used in the assay.

### FcRn-Targeted Nasal Immunization LED to Protection Against SARS-CoV-2 Infection.

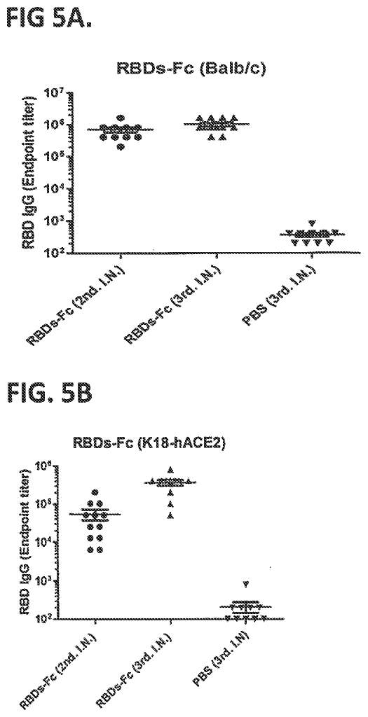

Human IgG1 Fc can bind to mouse FcRn. It was first tested whether FcRn-dependent respiratory transport augments the immune responses of the tandem RBD antigen. Human ACE2 transgenic mice under K18 promoter in a C57B/6 background were purchased from the Jackson Lab. Balb/c mice (FIG. 5A) or human ACE2 transgenic mice (N=10-12) (FIG. 5B) were intranasally (i.n.) immunized with purified 10 μg of the tandem RBDs-Fc/(FIG. 5A-B), or PBS along with 10 μg CpG, and boosted 2 weeks later with the same dose. The higher titers of RBD-specific IgG in sera were detected in the tandem RBDs-Fc immunized mice when compared with the PBS-treated groups of mice 2 weeks after the boost (FIG. 5A-B). It was found that the immunized Balb/c mice developed a much higher level of IgG antibody in comparison with that of the Human ACE2 transgenic mice after second nasal vaccination. Interestingly, in a third intranasal immunization, enhanced levels of IgG antibody was detected in human ACE2 transgenic mice, but not in the Balb/c mice, suggesting the Balb/c mice developed the highest level of RBD specific antibody after the second nasal immunization.

To test whether the immune responses elicited by FcRn-targeted intranasal vaccination provide protection, 2-3 weeks after the boost, Balb/c mice were infected with a mouse adapted SARS-CoV-2 virus MA10 (3×10e5 TCID50/mouse) or infected human ACE2 transgenic mice by SARS-CoV-2 variant Delta (2.5×10e4 TCID50). Mice were monitored and weighed daily for a 14-day period and were euthanized after 25% body weight loss as endpoint.

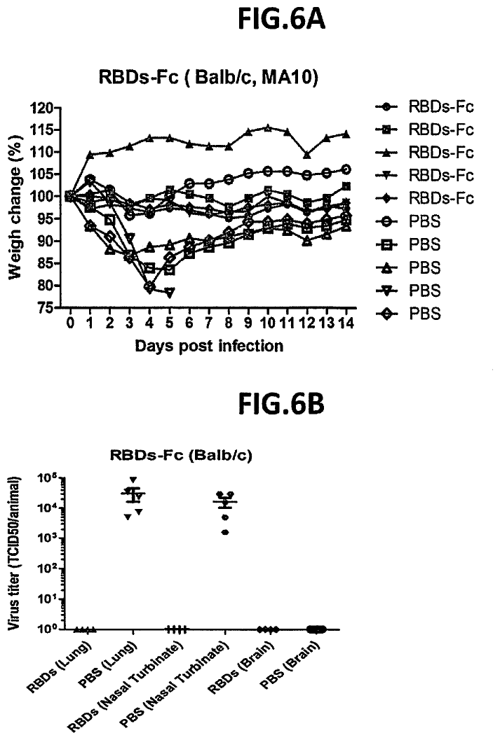

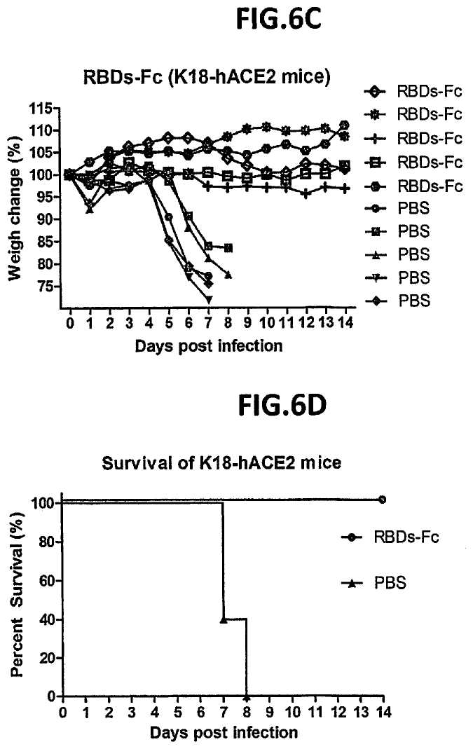

In Balb/c mice infected with MA10, the majority of mice in the PBS groups had slight weight loss within 8 days after the challenge and mice losing weight up to 25% either succumbed to infection or were euthanized. In contrast, all the tandem RBDs-Fc-immunized mice had no body-weight loss (FIG. 6A). In addition, virus replicating in different tissues 5 days after challenge (FIG. 6B) was assessed. Live virus was not detected in tissues, including nasal turbinate and lung of tandem RBDs-Fc-immunized mice. However, high level of titers of virus were detected in the nasal turbinate and lung of the PBS group (FIG. 6B), suggesting these control mice failed to contain viral replication in the respiratory tract.

In human ACE2 transgenic mice infected with Delta virus, all mice in the PBS groups had weight loss (up to 25%) within 7-8 days after the challenge and either succumbed to infection or euthanized (FIG. 6C). In contrast, all the tandem RBDs-Fc-immunized ACE2 transgenic mice had no body-weight loss. Hence, the tandem RBDs-Fc protein-immunized human ACE2 transgenic mice led to a full protection (FIG. 6D).

### FcRn-Targeted Nasal Immunization Shows a Cross-Neutralization Against SARS-CoV-2 Variants.

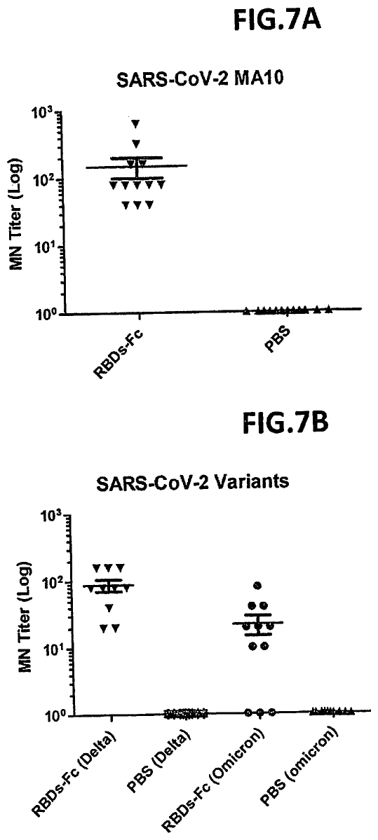

A major goal of using a tandem RBDs-Fc immunization strategy is to protect the host against SARS-CoV-2 and its variants. To demonstrate this, an in vitro microneutralization (MN) test was performed for measuring a neutralization antibody titer. The sera from the tandem RBDs-Fc (FIG. 1) immunized mice were incubated with SARS-CoV-2 (MA10), Delta variants, respectively. It was observed that the sera from the tandem RBDs-Fc (FIG. 1) immunized mice exhibited high neutralizing activity against SARS-CoV-2 and Delta variant relative to the PBS control groups (FIG. 7A-B). Although the tandem RBDs-Fc antigen (FIG. 1) does not contain Omicron-specific RBD, a microneutralization (MN) test was also performed using the Omicron variant. It was found that after the 3^{rd} dose intranasal immunization, the majority sera from the tandem RBDs-Fc (FIG. 1) immunized mice showed neutralizing activity against Omicron variant relative to the PBS control groups, although the neutralizing activity was not detected in the sera of 3 mice (FIG. 7B). This is reasonable because the tandem RBDs-Fc antigen (FIG. 1) used in this immunization does not contain Omicron-specific RBD.

Overall, the data disclosed herein demonstrates that FcRn-mediated nasal delivery of the tandem RBD-Fc conferred significant protection against lethal SARS-CoV-2 virus challenge, resulting in decreased mortality, viral replication, and most importantly, pulmonary inflammation. The coronavirus spike engages with the host cell receptor via its RBD to initiate viral entry. Using a similar strategy as disclosed above, tandem RBDs-Fc proteins displaying at least 12 RBDs from human and animal coronaviruses will be produced to evaluate whether the tandem RBD proteins can elicit cross-protective antibody and T cell responses. Hence, an FcRn-targeted nasal vaccination strategy using the tandem RBDs antigens derived from different human and animal coronaviruses can also be used to develop a pan-coronavirus vaccine against emerging coronaviruses that emerge in the future.

### Example 2

### Materials and Methods

### Cells, Antibodies, Viruses and Mice

Vero E6 (with high expression of endogenous ACE2, Cat No. NR-53726) and VAT (Vero E6-TMPRSS2-T2A-ACE2, Cat No. NR-54970) were from Biodefense and Emerging Infections Research Resources Repository (BEI Resources, Manassas, VA). Chinese hamster ovary (CHO) cells were purchased from the American Tissue Culture Collection (ATCC, Manassas, VA). Vero E6, VAT, and CHO cells were maintained in complete Dulbecco's Minimal Essential Medium (DMEM) (Invitrogen Life Technologies), both supplemented with 10% fetal bovine serum (FBS), 2 mM L-glutamine, nonessential amino acids, and antibiotic and antifungal (100 units/ml of penicillin, 100 μg/ml of streptomycin, and 250 ng/ml of amphotericin B). Vero E6, VAT, and CHO cells routinely tested negative for Mycoplasma sp. by real-time PCR. Recombinant CHO cells were grown in a complete medium with G418 (Invitrogen, 1 mg/ml). All cells were grown at 37° C. in 5% CO₂.

Different RBD proteins were from the BEI. Motavizumab, an antibody against the respiratory syncytial virus (RSV) F protein, was acquired from Cambridge Biologics (Brookline, MA). The horseradish peroxidase (HRP)-conjugated anti-human IgG (Cat #2081-05), anti-goat IgG (Cat #6160-05), and streptavidin (Cat #7100-05) were obtained from Southern Biotech (Birmingham, Alabama). HRP-conjugated anti-mouse IgG Fab (Cat #A9917) and anti-human IgG Fab (Cat #SAB4200791) were from Sigma. HRP-conjugated anti-mouse IgG (Cat #PA1-28568) was obtained from Invitrogen (Waltham, MA). Biotinylated human ACE2 protein (Cat #AC2-H82E6) was purchased from AcroBiosystems (Newark, DE). Human C1q protein was a gift from Dr. Sean Riley (Complement Technology, Cat #A099). Mouse C1q protein (Cat #M099) was procured from Complement Technology (Tyler, TX).

SARS-CoV-2 ancestral strain hCoV-19/USA/NY-PV08410/2020 (abbreviated as NY strain, Cat #NR-53514), Delta strain hCoV-19/USA/PHC658/2021(B.1.167.2) (Cat #NR-55611), and Omicron strain USA/MD-HP20874/2021(B.1.1.529) (Cat #NR-56461) were obtained from BEI Resources with the permission of the Centers for Disease Control and Prevention (CDC). Viruses from the BEI were passed in either Vero E6 (for NY and Delta strains) or VAT cells (for the Omicron strain). At 72 hr post-infection, tissue culture supernatants were collected and clarified before being aliquoted and stored at −80° C. The virus stock was respectively titrated by using TCID50. All virus experiments were performed in an approved Animal Biosafety Level 3+ (ABSL-3+) facility at the University of Maryland using appropriate positive-pressure air respirators and protective equipment.

The animals were acclimatized at the animal facility for 4-6 days before initiating experiments. Animals from different litters were randomly assigned to experimental groups, and investigators were not blinded to allocation during experiments and outcome assessment. Wild-type (WT) C57BL/6 mice were purchased from Charles River Laboratories (Frederick, MD). Transgenic mice expressing human ACE2 by the human cytokeratin 18 promoter (K18-hACE2) represent a susceptible rodent model. Specific-pathogen-free, 6-8-weeks-old, female and male B6.Cg-Tg(K18-ACE2)2Prlmn/J (Stock No: 034860, K18-hACE2) hemizygous C57BL/6 mice and control C57BL6 mice (non-carriers) were purchased from the Jackson Laboratory and used for breeding pairs to generate pups for research. All the offspring were subjected to genotyping, and only the hemizygous K18-hACE2 mice were chosen for future use. Animals have been maintained in individually ventilated cages at ABSL-2 for noninfectious studies or in isolators within the ABSL-3 facility for studies involving SARS-CoV-2 viruses. Immunizations and virus inoculations were performed under anesthesia. All mice were anesthetized with an intraperitoneal (i.p.) injection of fresh Avertin at 10-12.5 μl of working solution (40 mg/ml) per gram of body weight (Fisher Scientific) and laid down in a dorsal recumbent position to allow for recovery.

### Construction of Plasmids Expressing a SARS-CoV-2 RBDs and RBDs-Fc

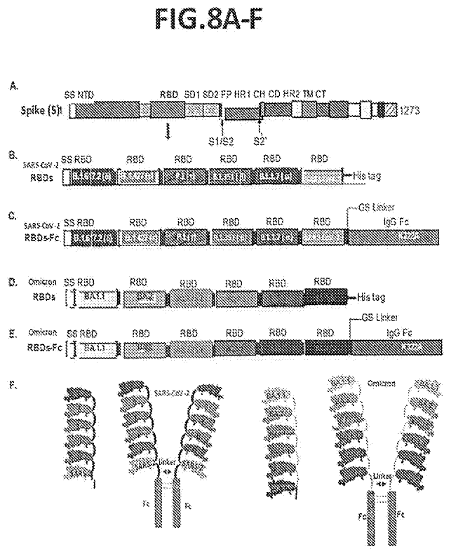

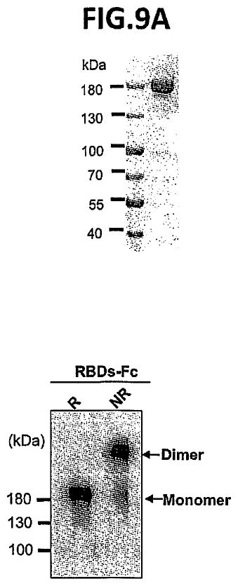

The entire amino acid (aa) sequence corresponding to the RBD of the Spike protein of the SARS-CoV-2 Wuhan-Hu-1 and variants strain a was retrieved from Genbank (MN908947.3). Tandem RBD cDNAs of SARS-CoV-2 variants or subvariants spaced with a linker were designed as described. To target FcRn for delivery, human IgG1 Fc was selected, which has the highest affinity for activating FcγRI, but the lowest affinity for inhibiting FcγRIIB. To prevent the C1q binding, the complement C1q-binding motif was mutated by replacing Lys322 with Ala (K322A) in IgG1 Fc (FIG. 8A). The RBDs-Fc gene was codon-optimized for optimal expression in CHO cells and synthesized in GenScript (Piscataway, NJ). and cloned into eukaryotic expression plasmid pcDNA3.1 via Kpn I and Xho I sites to generate the recombinant plasmid pcDNA3.1-RBDs-Fc (FIG. 8A). A control plasmid, pcDNA3.1-RBDs, was produced by replacing the human IgG1 Fc portion with the 6×His tag sequence.

### Expression and Characterization of the Tandem RBDs and RBDs-Fc Fusion Proteins

The pCDNA3.1 plasmids encoding RBDs-Fc or RBDs were transfected into CHO cells using PEI MAX 40000 (Fisher Scientific, Cat #NC1038561). Stable cell lines were selected and maintained under G418 (1 mg/ml). Expression and secretion of RBDs-Fc or RBDs fusion proteins were determined by immunofluorescence assay, SDS-PAGE, and Western blotting analysis. The soluble RBDs-Fc or RBDs proteins were produced by culturing CHO cells in a complete medium containing 5% FBS with ultra-low IgG. The proteins were captured by Protein A column (ThermoFisher Scientific, Cat #20356) for the RBDs-Fc protein or Histidine-tagged Protein Purification Resin (R&D Systems, Cat #1P999) for the RBDs protein, eluted with 0.1M Glycine (pH 2.5), and neutralized with 1M Tris-HCl (pH8.0). Glycine and Tris-HCl in the protein solution were replaced with PBS three times using centrifugation with Amicon Ultra-15 Centrifugal Filter Unit (50K) (Millipore, Cat #UFC905024). Protein concentrations were determined using a NanoDrop spectrophotometer (Thermo Scientific).

### SDS-PAGE Gel and Western Blotting

Protein quality was assessed by 8-12% SDS-PAGE gel under reducing conditions. Proteins in gels were either stained with Coomassie blue dye in gel or used for transfer onto nitrocellulose membranes (GE Healthcare). The membranes were blocked with 5% milk in PBST (PBS and 0.05% Tween-20) and incubated with appropriate primary and HRP-conjugated secondary antibodies, as indicated in the Figure legends. The immobilon Western chemiluminescent HRP substrate (Millipore, Cat #WBKLS0100) was used to visualize protein bands in membranes and images captured by the Chemi Doc XRS system (BioRad).

### Enzyme-Linked Immunosorbent Assay (ELISA)

For the detection of SARS-CoV-2 RBD-specific antibodies IgA and IgG in sera collected along the detection timeline after the immunizations, 96-well plates (Maxisorp, Nunc) were coated with 1 μg/ml of the different RBD proteins as described above in 100 μl coating buffer (PBS, pH7.4) per well and incubated overnight at 4° C. Plates were then washed four times with 0.05% Tween 20 in PBS (PBST) and blocked with blocking buffer (2% bovine serum albumin in PBST) for 2 hr at room temperature. The serially diluted sera from animals were added to each well and incubated for 2 hrs. After washing six times with PBST, the detection antibodies were added and incubated for 1.5 hr at room temperature. HRP-conjugated rabbit anti-mouse IgG (1:20,000, Invitrogen, Cat #PA1-28568) was used for measuring mouse IgG, while biotin-labeled goat anti-mouse IgA Ab (1:5000, Southern Biotech, Cat #1040-08) plus HRP-conjugated streptavidin (1:70000, Southern Biotech, Cat #7100-05) were used for measuring mouse IgA antibody. 100 μl TMB (tetramethyl benzidine) (BD, Cat #555214) was used as the substrate to visualize the signals. Reactions were stopped with 100 μl of 1 M sulfuric acid. Optical density at 450 nm was determined using a Victor III microplate reader (Perkin Elmer). Titers represent the reciprocal of the highest dilution of samples showing a 2-fold increase over the average OD₄₅₀ nm values of the blank wells.

The ELISA assays were used to measure interactions of human ACE2 (ACROBiosystems, Cat #AC2-H82E6); and human and mouse C1q protein (Complement Technology, Cat #A099 and M099). To facilitate detection, all ACE2 and C1q proteins were conjugated with biotin. In brief, ELISA plates were coated with RBDs-Fc or RBDs protein in PBS (200 ng/well for ACE2 binding) overnight at 4° C. After blocking for 2 hr, the 2-fold serial diluted target proteins (0.4-400 ng/ml of hACE2) were added and incubated for 2 hr at room temperature. For the C1q binding assay, RBDs-Fc or RBD proteins were used to coat plates at a serial dilution (800-7.8 ng/well), and a biotin-conjugated human or mouse C1q (2 μg/ml) was used for detection. For all assays, the streptavidin-HRP (1:5000) and TMB were used to visualize the colorimetric signals.

### Quantification of SARS-CoV-2 Virus

The number of infectious virus particles in the specimen of ancestral SARS-CoV-2 or Delta strain infected animals was determined in Vero E6 cells by 50% tissue culture infectious dose (TCID₅₀) endpoint dilution assay as described. The quantification of the Omicron strain was performed in VAT cells. To increase the sensitivity, VAT cells were also used in detecting ancestral viruses and Omicron variants in the throat swab samples. The overexpression of the hACE2 and TMPRSS2 in VAT cells enhances the replication efficiency of the SARS-CoV-2. Briefly, cells were plated at 15,000 cells/well in DMEM with 10% FBS and incubated overnight at 37° C. with 5.0% CO₂. Media was aspirated and replaced with DMEM with 1% inactivated FBS for virus infection. Animal tissues including nasal turbinate, lung, brain, intestine, and kidney were homogenized in the TissueLyser LT (Qiagen). After centrifuging at high speed (14000 rpm, 10 min), the 10-fold serial dilutions of supernatants were used to infect the cell monolayers in 96 well plates, and the CPE was checked after four days. Positive (virus stock of known infectious titer) and negative (medium only) controls were included in each assay. The virus titer was expressed as TCID₅₀/ml (50% infectious dose (ID₅₀) per milliliter) by using the Reed-Muench method.

### Microneutralization (MN) Assay

Neutralizing antibodies were measured by a standard microneutralization (MN) assay on Vero-E6 (for ancestral and Delta strains) or VTA cells (for Omicron strain) as previously described. The sera were heat-inactivated at 56° C. for 30 min and followed by 2-fold serial dilution, after which the diluted sera were incubated with 100 TCID₅₀ of SARS-CoV-2 virus (ancestral, Delta, and Omicron strains) for 1 hr at 37° C., respectively. The virus-serum mixtures were added to Vero-E6 or VAT cell monolayers in 96-well plates and incubated for 1 hr at 37° C. After removing the mixture, DMEM with 1% inactivated FBS was added to each well and incubated for four days at 37° C. for daily CPE observation. Neutralizing Ab titers are expressed as the reciprocal of the highest serum dilution preventing the appearance of CPE.

### SARS-CoV-2, SARS-CoV-1, and MERS-CoV Pseudovirus Inhibition Assays

Pseudovirus inhibition assays were performed in Dr. Lanying Du laboratory to detect the neutralizing activity of immunized mouse sera against infection of SARS-CoV-2, SARS-CoV-1, and MERS-CoV pseudovirus in target cells. Briefly, pseudovirus-containing supernatants were respectively incubated with serially diluted mouse sera at 37° C. for 1 h before adding to target cells replated in 96-well culture plates (10⁴ cells/well). 24 hr later, cells were incubated with fresh medium, which was followed by lysing cells 72 h later using cell lysis buffer (Promega) and transferring the lysates into 96-well luminometer plates. Luciferase substrate (Promega) was added to the plates, and relative luciferase activity was determined. The inhibition of SARS-CoV-2, SARS-CoV-1, and MERS-CoV pseudoviruses was presented as % inhibition.

### Immunizations of Mice and SARS-CoV-2 Virus Infection

Six to eight-week-old female/male C57BL/6 mice, FcRn KO mice, and K18-hACE2 transgenic mice were intranasally (i.n.) immunized with 10 μg RBDs-Fc, equal molar of RBDs, or PBS in 10 μg CpG adjuvant (ODN1826, Invivogen, Cat #vac-1826-1) in a total volume of 20 μl. For intramuscular (i.m.) immunizations, mice were injected bilaterally in the quadriceps femoris with a 50 μl volume containing 10 μg RBDs-Fc or RBDs antigen in 10 g CpG. The mice were boosted with the same vaccine formulations two or three weeks later.

Two or three weeks after the boost, blood was collected from each animal; 3 days later, the animals were transferred to the ABSL-3+ facility for virus challenge. The K18-hACE2 mice were i.n. infected with lethal doses of SARS-CoV-2 virus in a total volume of 50 μl (2.5×10⁴ TCID₅₀ for ancestral and Delta strains). After infection, animals were monitored daily for morbidity (weight loss), mortality (survival), and other clinical signs of illness for 14 days. Animals losing above 25% of their body weight following infection or reaching the humane endpoint were humanely euthanized.

To further measure the virus replication and tissue lesion in vivo, 50% of the animals in each group were euthanized at 4 or 5 days post-infection (dpi) and different organs and tissues, including nasal turbinate, lung, and brain were harvested. The left lung lobe was fixed in a 10% neutral buffered formalin solution for histopathology analyses, while the right lung lobes and other tissues were homogenized in DMEM by Tissue Lyser (Qiagen). The homogenates were cleaned by centrifugation (15000 rpm for 10 minutes), and supernatants were collected to measure viral load.

### Lung Pathology

To examine the lung pathology, lungs were removed from mice in each group and fixed in 10% neutral buffered formalin solution three days before transferring the tissues out of the ABSL-3 facility. The lungs were then paraffin-embedded, sectioned in five-micron thickness, and stained with Hematoxylin and Eosin (H & E) by Histoserv Inc (Germantown, MD). Stained lung sections were scanned using a high-definition whole-slide imaging system (Histoserv, Germantown, MD).

To determine the level of pulmonary inflammation, the lung inflammation was evaluated and scored by a board-certified veterinary pathologist blinded to the experimental design. A semi-quantitative scoring system, ranging from 0 to 5, was used to assess the following parameters: alveolitis, parenchymal pneumonia, inflammatory cell infiltration, peribronchiolitis, perivasculitis, and lung edema. The inflammatory scores are as follows: 0, normal; 1, very mild; 2, mild; 3, moderate; 4, marked; and 5, severe. An increment of 0.5 was assigned if the inflammatory score fell between two.

### Statistics Analysis

All data were analyzed with the Prism 9.0 software (GraphPad). The student t-test was used to compare the means between two groups, while One-way ANOVA was used to compare if three or more groups were involved. Meanwhile, a Post Hoc test was applied after One-way ANOVA. Dunnett's multiple comparisons test was used to compare means from different treatment groups against a single control group. The Turkey test was performed to identify the difference between the two groups. To compare the Kaplan-Meier survival curves, the Mantel-Cox test was used. Fisher's exact test was conducted for comparisons of transmission capacities among various groups. All statistical methods used in each experiment are indicated in the Figure legends. The level of statistical significance was assigned when P values were <0.05. The statistical significance was further classified as four levels: * (P<0.05), ** (P<0.01), *** (P<0.001), and **** (P<0.0001).

### Results

### Expression and Characterization of Tandem RBDs-Fc Fusion Proteins

To target RBDs antigen to FcRn, a human IgG1 Fc fused to a tandem RBDs was produced (FIG. 8A). Since complement activation contributes to a cytokine storm in COVID-19 patients (Yan B 2021), the C1q binding site of human IgG1 Fc was removed. It was shown that the soluble RBDs or RBDs-Fc protein was secreted from the stable CHO cells (FIG. 8A-B). A functional test of the Fc-domain was confirmed in vitro by immunoprecipitation with Staphylococcal Protein A beads for RBDs-Fc. It has been shown that Protein A makes contact with overlapping amino acids of IgG Fc for binding to the FcRn. Therefore, this suggests the Fc portion of IgG on the RBDs-Fc maintains all structures necessary for binding FcRn.

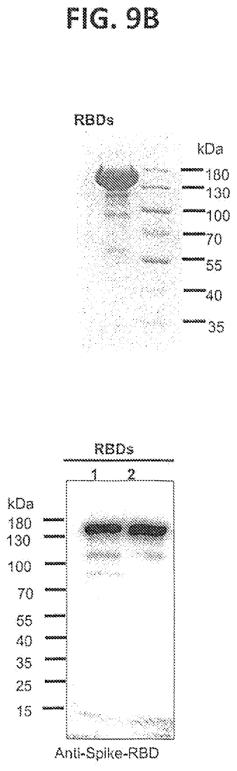

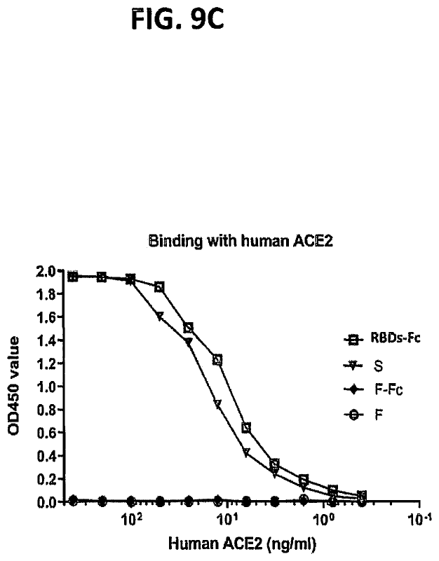

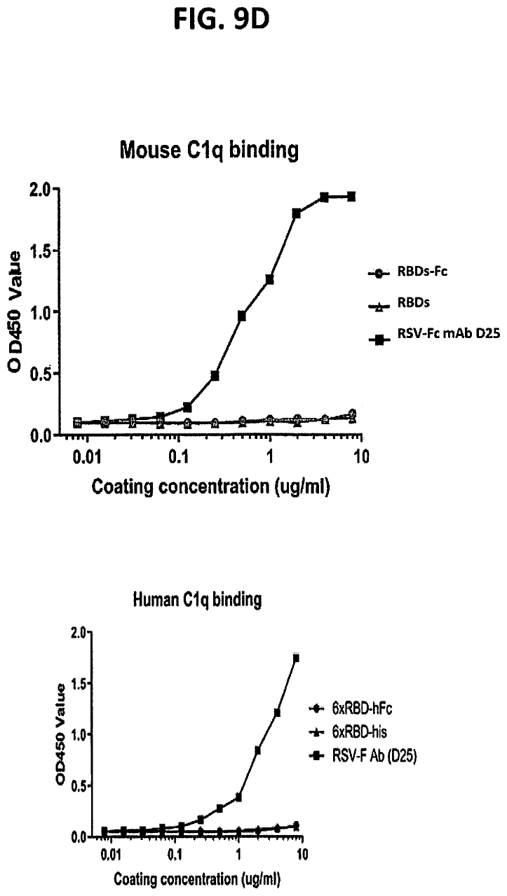

It was next determined if the RBD portion of the RBDs-Fc binds to human ACE-2. In an ELISA assay, the RBDs-Fc and S protein bound human ACE-2 similarly (FIG. 9C), indicating the Fc fusion doesn't affect the conformation of RBDs or S protein for bonding to ACE2 in the RBDs-Fc protein. As a negative control, RSV F protein with or without Fc-fusion did not bind to human ACE-2 (FIG. 9C). In contrast to an RSV-F specific mAb (D25), the RBDs-Fc could not bind to human or mouse C1q protein (FIG. 9D) in vitro.

Human Neutralizing mAbs Interacted with the RBDs-Fc

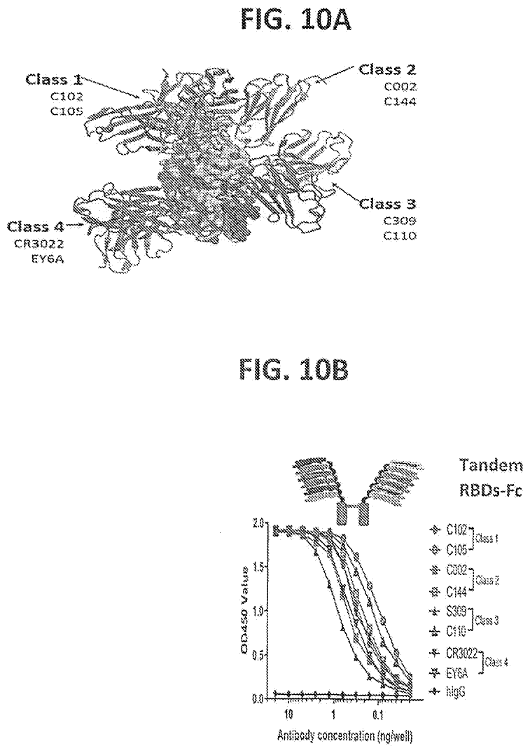

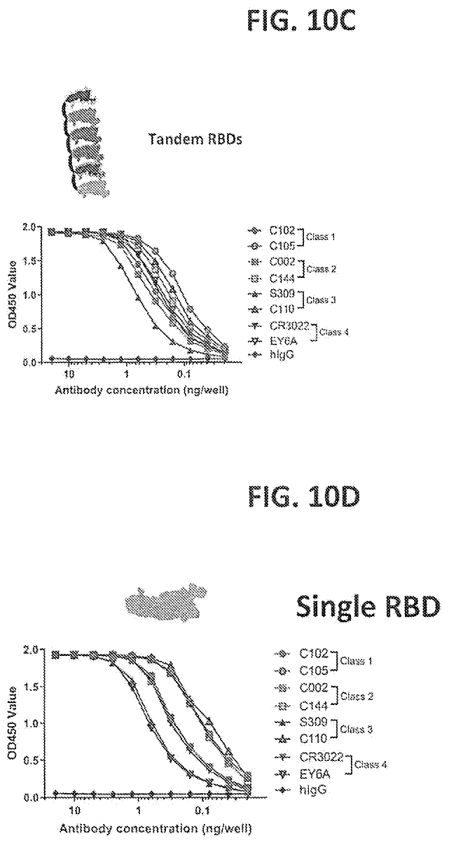

There are several major non-overlapping antigenic sites on the RBD. Most RBD-directed neutralizing antibodies, including C144 (FIG. 10A), recognize the tip region of the RBD, and they often show remarkable potency in blocking ACE2 engagement by direct competition. Another site lies on the exposed surface of the RBD when it is in the down configuration and is targeted by antibodies such as S309. The third site often referred to as a ‘cryptic supersite’, targeted by CR3022, is on the buried side of the RBD and is fully accessible only when the domain is in the up conformation. Together, these sites consist of the epitopes of most neutralizing antibodies to SARS-CoV-2. To test if the RBD portion of the RBDs-Fc interacts with human mAbs compared to the RBDs or RBD alone experiments were done. Normal human IgG was used as a negative control. Indeed, the RBD-specific human mAbs were able to equivalently recognize both purified RBDs-Fc (FIG. 10B), RBDs (FIG. 10C), and RBD (FIG. 10D) protein. Most importantly, RBDs-Fc and RBDs showed similar levels of binding for each tested human mAb, indicating Fc-fusion with tandem didn't alter its conformation. Normal human IgG showed no binding activity. Together, the RBDs portion of the RBDs-Fc protein maintains the correct conformational structure allowing for binding to the ACE-2 and RBD-specific human mAbs.

Antibody Induced by Intranasal (i.n.) Immunizations with the Tandem RBDs-Fc Interacted with RBD Proteins Derived from SARS-CoV-2 Variants.

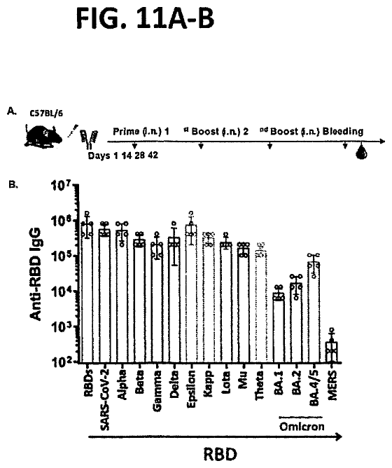

Because of the tandem character, it was tested whether serum IgG antibody induced by intranasal (i.n.) immunizations with the tandem RBDs-Fc can interact with RBD proteins derived from SARS-CoV-2 variants. Therefore, C57BL/6 mice were i.n. immunized with 10 μg of RBDs-Fc in 10 μg CpG adjuvant, the mice were boosted twice in a two-week interval (FIG. 11A). An ELISA experiment was performed by coating purified RBD proteins from different SARS-CoV-2 variants, as indicated in FIG. 11B. The purified RBDs protein was used as a positive control. Serum IgG antibodies reacted strongly with the tandem RBDs protein (FIG. 11B). It was also found that serum IgG antibodies recognized individual RBD proteins from ancestral SARS-CoV-2, α, β, γ, δ and ε, which were displayed in RBDs-Fc (FIG. 8C). Interestingly, IgG antibody in the sera also interacted strongly with individual RBD protein from SARS-CoV-2 variants kappa, lota, mu, and theta, which were not displayed in RBDs-Fc (FIG. 8C). Furthermore, the serum IgG antibody also reacted with Omicron subvariants BA.1, BA.2, and BA.4/5, although the IgG binding to BA.1 or BA.2 RBD had a weak activity in comparison with that of BA.4/5 RBD. This is not surprising, because the RBD of SARS-CoV-2 Omicron variant comprises the most significant number of mutations. In addition, the mutations in RBD are associated with a significantly increased ability to evade vaccine-induced immunity. However, these data suggest that the serum IgG antibody from the RBDs-Fc (FIG. 8C) immunized mice have a broad immune response. As expected, the serum IgG reacted barely with MERS-CoV RBD.

### Intranasal Vaccination by the RBDs-Fc Protein Leads to Increased Protection Against Infection by the SARS-CoV-2 and its Variants in K18-hACE2 Transgenic Mice

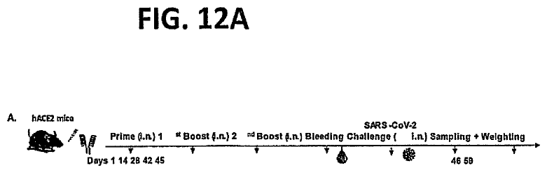

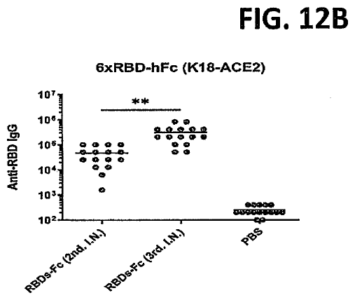

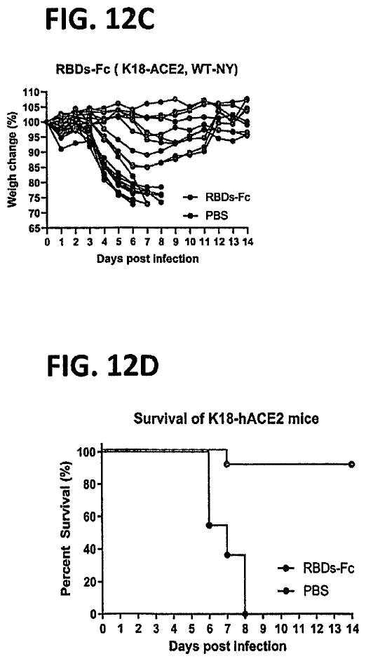

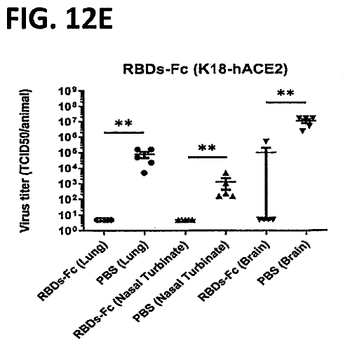

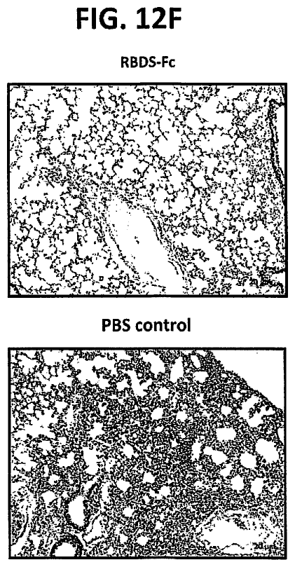

Transgenic mice expressing the human ACE2 under the control of the K18 promoter, when experimentally infected with SARS-CoV-2, developed respiratory disease resembling severe COVID-19. They are highly susceptible to SARS-CoV-2 intranasal challenges when high virus doses are used. To show whether the tandem RBDs-Fc protein can induce protective immunity in vivo, K18-hACE2 mice were i.n. immunized with 10 μg of RBDs-Fc or PBS in 10 μg CpG and boosted twice in a 2-week interval (FIG. 12A). We confirmed that significantly higher titers of serum IgG (FIG. 12B) were detected in the K18-hACE2 mice that were i.n. immunized with the S-Fc protein when compared with PBS control mice. To test whether the immune responses elicited by the intranasal immunization with the RBDs-Fc protein provide protection, we i.n. challenged all immunized mice with a lethal dose (2.5×10⁴ TCID₅₀) of ancestral SARS-CoV-2 virus 2 weeks following the last boost (FIG. 12A). Mice were monitored and weighed daily for 14 days. Once a mouse lost 25% or more of their initial body weight, they were euthanized. Most of the mice in the PBS control groups exhibited rapid weight loss following the challenge, either succumbing to infection within 8 days post-infection or subjecting to euthanasia. In contrast, the majority of RBDs-Fc-immunized ACE2 mice did not experience significant body weight loss (FIG. 12C), and only 1 immunized mouse had body weight loss. Hence, 90% of the RBDs-Fc immunized mice had full protection with significantly higher survival rates than those of the PBS control group (FIG. 12D). As expected, a significant reduction of virus load in the nasal turbinate, lungs, and brain tissue was detected in most RBDs-Fc immunized mice when compared to the PBS control animals (FIG. 12E). Interestingly, brain tissues exhibited high levels of virus load in the PBS control mice, we detected the live virus in the brain of only one of the RBDs-Fc-immunized mice. Therefore, the RBDs-Fc-immunized hACE2 transgenic mice essentially contained viral replication in tissues/organs of the viral entry and prevented the viral spreading to other tissues/organs, including the central nervous system.

To further show protection in the respiratory tract, lung tissues were collected five days following the challenge for histopathological analysis. The lungs of uninfected mice were used as normal control. No apparent alterations were observed in the lung structure of the RBDs-Fc immunized mice (FIG. 12F). In contrast, focal inflammatory cell infiltration, pneumonia, peribronchiolitis, and perivasculitis in the lungs of PBS control mice was found (FIG. 12F). However, alveolitis was rarely observed. Therefore, the mice immunized with RBDs-Fc had a significantly lower inflammation score of the lungs compared with those of mice in the PBS control mice. These findings demonstrate that the RBDs-Fc immunization confers significant protection against the lethal SARS-CoV-2 challenge, resulting in decreased mortality, viral replication, and pulmonary inflammation in the human ACE2 transgenic mouse model.

Intranasal Vaccination with the RBDs-Fc Protein Leads to Protection Against SARS-CoV-2 Variants

SARS-CoV-2 is rapidly evolving via mutagenesis, which significantly impacts transmissibility, morbidity, reinfection, and mortality. Six variants of SARS-CoV-2 named variants of concern (Alpha to Omicron) have been identified and reported. The Delta variant used to be the dominant strain, but the Omicron strain has become the most prevalent and contagious variant worldwide. Omicron strain can bind the human ACE2 receptor with increased transmissibility and manifests many immune escape strategies in natural infections or against immune responses induced by current vaccines. Since the RBD portion of the tandem RBDs-Fc is derived from SARS-CoV-2 variants (FIG. 8C) or Omicron subvariants (FIG. 8E), we are interested in testing the effectiveness and neutralizing activity elicited by the RBDs-Fc vaccine against Delta in this study and Omicron subvariants in the future.

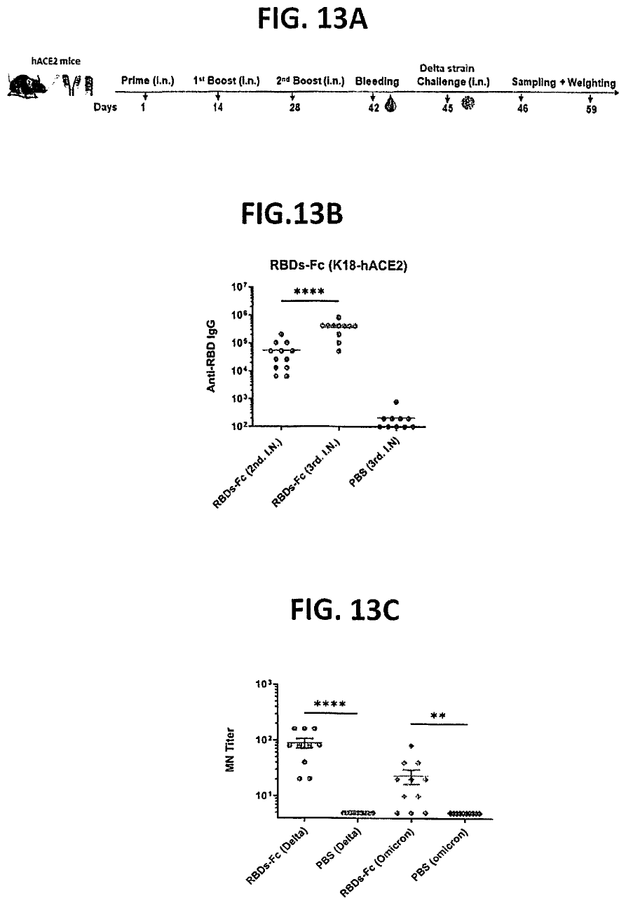

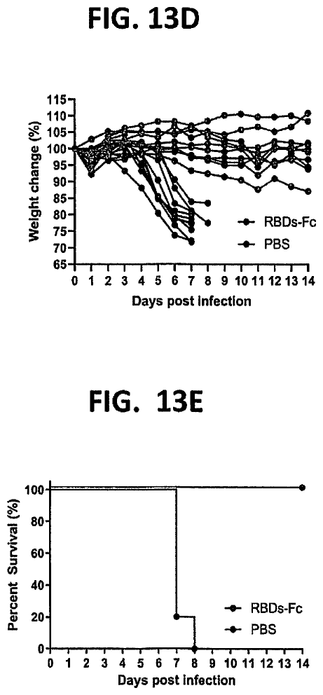

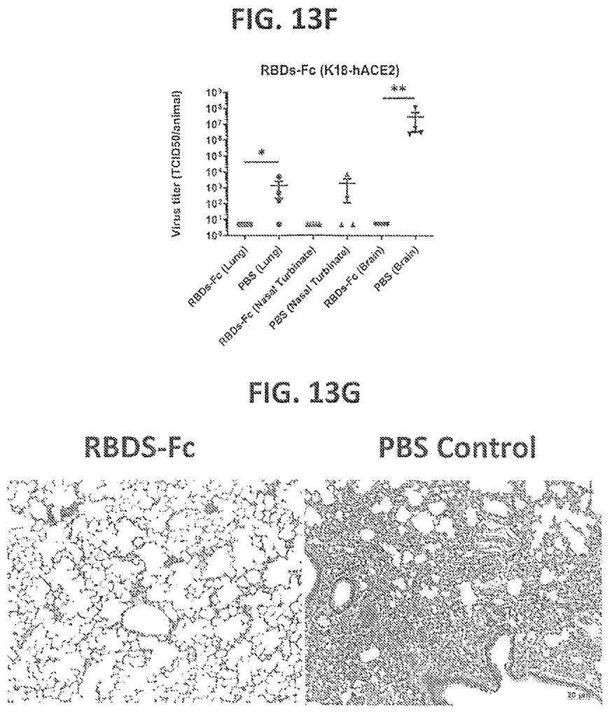

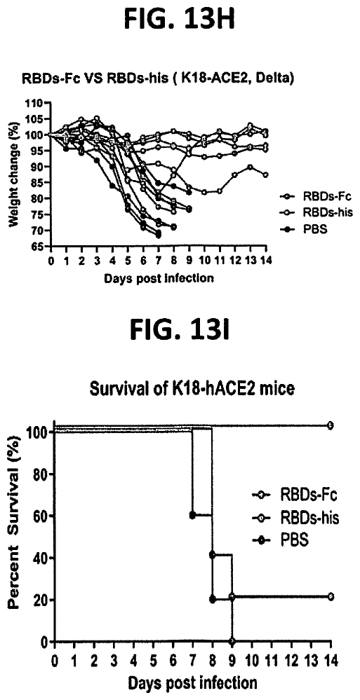

Once again, hACE2 mice were immunized with 10 μg of RBDs-Fc adjuvanted in g CpG (FIG. 13A). In comparison with that of the PBS control mice, the tandem RBDs-Fc immunized mice developed a significant level of IgG antibodies in their sera after their first and send boost (FIG. 13B). Most of the immunized mice developed neutralizing antibodies against the Delta or Omicron A.1 strain of SARS-CoV-2 virus after the boost (FIG. 13C). To show protection, all immunized mice were challenged with a lethal dose (2.5×10⁴ TCID₅₀) of the Delta strain of SARS-CoV-2 virus 17 days following the boost. All mice in the PBS control groups experienced rapid weight loss, either succumbing to viral infection or euthanasia (FIG. 13D). In contrast, the RBDs-Fc-immunized mice did not show significant body-weight loss (FIG. 13D). The 100% of the RBDs-Fc-immunized mice survived, which was significantly higher than the survival rates of the PBS control groups where all mice died from Delta virus infection (FIG. 13E). Further, viral replication in the nasal turbinate, lung, and brain was measured six days post-infection (FIG. 13F). Live Delta virus was detected in the nasal turbinate, lung, and brain tissues of the PBS control mice, but there was a failure to find any live virus in the above tissues of the tandem RBDs-Fc-immunized ACE2 mice (FIG. 13F). No prominent inflammation was observed in the lungs of the RBDs-Fc immunized mice (FIG. 13G, left panel). In contrast, focal perivascular and peribronchial inflammation and thickened alveolar septa were found in the lung of PBS control mice (FIG. 13G, right panel). Further, the mice immunized with the tandem RBDs-Fc had a significantly lower inflammation score of the lungs compared with those of mice in the control and challenged animals.

To show whether the tandem RBDs without Fc can induce protective immunity to resist the infection of SARS-CoV-2 Delta virus, groups (n=5) of human ACE2 transgenic mice were i.n. immunized with 10 μg of RBDs-Fc, RBDs protein (equal molar amount), or PBS in 10 g CpG adjuvant, the mice were boosted twice in a two-week interval. To test whether the immune responses elicited by the intranasal immunization with the RBDs protein provide protection, all immunized mice were i.n. challenged with a lethal dose (2.5×10⁴ TCID₅₀) of SARS-CoV-2 Delta virus 2 weeks following the last boost. Mice were monitored and weighed daily for 14 days. Like the PBS control group, 80% of the mice in the RBDs immunized groups exhibited rapid weight loss or died following the infection (FIG. 13H). In contrast, all RBDs-Fc-immunized ACE2 mice did not have significant body weight loss. These results indicated that the i.n. immunization of tandem RBDs alone failed to induce full protection (FIG. 13I); the fusion of Fc to RBDs is required to induce protective immune responses through intranasal immunization.

Sera from Mice Intranasally Immunized by the Tandem RBDs-Fc Neutralize Infections of SARS-CoV-1, SARS-CoV-2, and MERS

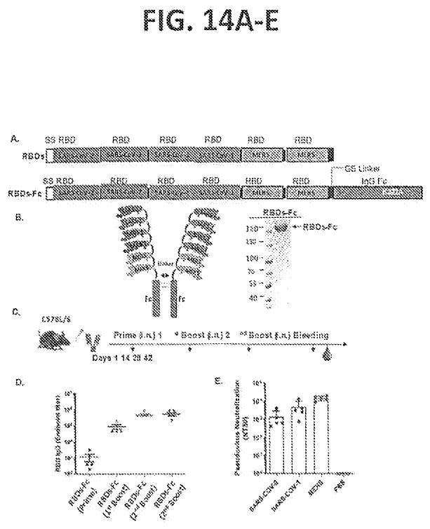

To further test whether the i.n. immunization using the tandem RBDs-Fc protein strategy can protect coronavirus infections beyond SARS-CoV-2, a protein expression plasmid was constructed consisting of tandem RBDs derived from SARS-CoV-2, SARS-CoV-1, and MERS-CoV (middle east respiratory syndrome coronavirus) (FIG. 14A). To target RBDs antigen to FcRn, a human IgG1 Fc fused to this tandem RBDs (FIG. 14A, 14B). Similarly, the complement C1q binding site of human IgG1 Fc was removed and produced a non-lytic vaccine antigen. The soluble RBDs or RBDs-Fc protein was secreted from the stable CHO cells (FIG. 14B). A functional test of the Fc-domain was confirmed in vitro by immunoprecipitation with Staphylococcal Protein A beads for RBDs-Fc. It has been shown that Protein A makes contact with overlapping amino acids of IgG Fc for binding to the FcRn. This result suggests the Fc portion of IgG on the RBDs-Fc maintains all structures necessary for binding FcRn.

To show whether RBDs-Fc immunized mice develop RBD-specific neutralizing antibody against SARS-CoV-2, SARS-CoV-1, and MERS-CoV, i.n. immunized C57BL/6 mice (n=5) were tested with 10 μg of RBDs-Fc adjuvanted in 10 μg CpG (FIG. 14C). In comparison with that of the PBS control mice, the tandem RBDs-Fc immunized mice developed a significant level of IgG antibodies in their sera after the first and second boost (FIG. 14D). In a pseudovirus assay in Dr. Lanying Du's laboratory, all immunized mice developed neutralizing antibodies against SARS-CoV-2, SARS-CoV-1, and MERS-CoV after the boost (FIG. 14E). These results demonstrate that the i.n. immunization with RBDs-Fc can induce neutralizing antibodies against at least three different coronaviruses in one vaccine antigen. An in vivo immunization experiment is being performed to show whether the tandem RBDs-Fc can provide protection in animal models. These data suggest that the immunization with the tandem RBDs-Fc strategy can also be used to develop a pan-coronavirus vaccine in the future.

### Intramuscular Immunization by the Tandem RBDs Induces RBD Specific IgG Immune Responses.

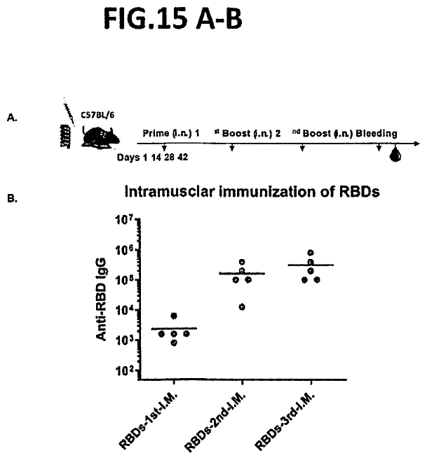

As demonstrated in FIG. 13H and FIG. 13I, the majority of ACE2 mice that were i.n. immunized by tandem RBDs were unable to develop a protective immune response against infection of SARS-CoV-2 Delta variants. Very likely, this is caused by the respiratory epithelial barrier that blocks the transport of tandem RBDs across the respiratory barrier and engages with immune cells under the respiratory epithelial barrier. To show whether the C57BL/6 mice that are intramuscularly (i.m.) immunized by tandem RBDs are capable of developing RBD-specific antibody immune response, we i.m. immunized C57BL/6 mice (n=5) with 10 μg of RBDs-Fc adjuvanted in 10 μg CpG (FIG. 15A). In an ELISA test, the tandem RBDs immunized mice developed a significant level of RBD-specific IgG antibodies in their sera after the first and second boost (FIG. 15B). These results suggest that the tandem RBDs alone can induce a strong antibody immune response through an intramuscular injection by protein or vector-based expression or an intranasal immunization through a vector-mediated delivery.

RBDs-Delta DNA
(SEQ ID NO. 1)
ATGTTCGTGTTTCTGGTGCTGCTGCCTCTGGTGTCCAGCCAGTGCGTGAGGGTGCAGCCAACCGAGAGCATCGTG
CGGTTCCCAAACATCACAAATCTGTGCCCCTTCGGCGAGGTGTTTAACGCCACCCGGTTTGCTTCCGTGTACGCC
TGGAACAGGAAGCGGATCTCCAATTGCGTGGCCGACTACAGCGTGCTGTATAATTCCGCTAGCTTCTCTACATTT
AAGTGCTATGGCGTGTCTCCCACCAAGCTGAACGATCTGTGCTTCACAAACGTGTACGCCGACTCCTTTGTGATC
AGGGGAGATGAAGTGAGGCAGATCGCTCCAGGACAGACCGGCAAGATCGCTGACTACAACTATAAGCTGCCCGAC
GATTTCACAGGCTGCGTGATCGCTTGGAATTCTAACAATGTGGATTCCAAAGTGGGCGGCAACTACAATTATAGA
TACCGCCTGTTCAGAAAGAGCAACCTGAAGCCTTTTGAGCGCGACATCAGCACCGAGATCTATCAGGCCGGCTCT
AAGCCATGCAACGGCGTGGAGGGCTTCAATTGTTACTTTCCACTGCAGTCTTATGGCTTCCAGCCCACAAACGGC
GTGGGCTACCAGCCTTATAGGGTGGTGGTGCTGAGCTTTGAGCTGCTGCACGCTCCAGCTACCGTGTGCGGACCC
AAGAAGTCTACAAACCTGGTGAAGAATAAATGCGTGAACTTCGGATCCGGAAGCGGCTCTAGGGTGCAGCCTACC
GAGTCCATCGTGCGGTTTCCTAATATTACAAATGTGTGCCCATTCGGCGAAGTCTTTAACGCTACCGGGTTCGCC
AGCGTGTACGCTTGGAATAGAAAGCGGATCTCTAACTGCGTGGCTGACTACTCCGTGCTGTACAACTCCGCTTCT
TTTTCTACATTTAAATGTTATGGCGTGTCCCCAACTAAACTGAATGACCTGTGCTTTACCAACGTGTACGCCGAC
AGCTTTGTGATCAGAGGCGACGAAGTCAGACAGATCGCTCCTGGCCAGACTGGCAAGATCGCTGACTATAACTAC
AAGCTGCCAGACGACTTCACCGGTTGCGTGATTGCTTGGAATAGCAACAATCTGGATTCTAAAGTGGGGGGCAAC
TATAATTACAGATATCGCCTGTTCAGAAAGTCCAACCTGAAGCCCTTTGAGCGCGATATCTCTACTGAGATCTAT
CAGGCTGGCAGCACACCTTGTAATGGTGTCGAAGGTTTTAACTGCTATTTTCCACTGCAGAGCTATGGCTTCCAG
CCAACCAACGGAGTCGGCTACCAGCCATATAGAGTGGTGGTGCTGTCTTTTGAGCTGCTGCATGCTCCTGCTACC
GTGTGCGGACCAAAGAAGTCCACAAATCTGGTGAAGAACAAGTGGGTGAATTTGGGATCTGGCTCTTCCGGAAGA
GTCCAGCCAACTGAATCCATCGTGCGGTTTCCCAATATTACCAATCTGTGCCCTTTCGGCGAAGTCTTCAACGCC
ACCCGCTTCGCCTCCGTGTACGCTTGGAACAGAAAACGGATCTCTAATTGCGTGGCTGACTACTCTGTGCTGTAC
AACTCCGCTAGTTTTTCTACATTTAAGTGTTACGGCGTGAGCCCTACTAAACTGAATGATCTGTGCTTTACTAAC
GTGTACGCTGACTCTTTTGTGATTAGAGGCGACGAAGTCCGCCAGATTGCCCCAGGACAGACCGGCACAATTGCC
GATTATAACTATAAGCTGCCTGACGACTTCACCGGATGCGTGATTGCCTGGAACTCCAACAATCTGGATAGTALA
GTCGGTGGCAACTACAATTATCTGTACAGGCTGTTCCGCAAGTCCAACCTGAAGCCATTTGAGAGGGACATCTCT
ACCGAGATCTACCAGGCTGGATCCACACCATGCAACGGAGTGAAAGGTTTTAACTGCTACTTCCCACTGCAGTCA
TACGGCTTCCAGCCAACCTACGGAGTGGGCTACCAGCCATACAGAGTCGTGGTGCTGAGCTTTGAGTTACTGCAT
GCCCCCGCCACCGTGTGCGGACCTAAGAAGAGCACCAATCTGGTCAAAAACALATGCGTGAACTTCTCTGGAGGA
TCCGGCAGCAGAGTGCAGCCAACCGAGTCCATCGTGCGCTTTCCTAATATCACCAACCTGTGCCCCTTCGGCGAA
GTGTTTAACGCTACCAGATTTGCTAGCGTGTACGCATGGAATAGAAAGCGCATTTCTAACTGCGTGGCAGATTAT
AGCGTGCTGTATAATTCTGCTTCCTTCAGCACATTTAAATGTTACGGCGTGTCCCCTACTAAACTGAACGACCTG
TGCTTTACAAACGTGTACGCTGATAGCTTTGTGATCAGAGGTGACGAAGTCCGCCAGATCGCCCCTGGCCAGACC
GGCAACATCGCTGACTATAATTACAAGCTGCCCGACGACTTCACCGGGTGCGTGATTGCTTGGAACAGCAATAAC
CTGGACAGCAAAGTCGGTGGTAACTATAATTACCTGTATAGACTGTTCCGCAAGTCTAATCTGAAGCCTTTTGAG
AGAGATATCAGCACCGAGATCTACCAGGCCGGCTCTACACCATGCAACGGCGTGAAGGGCTTCAATTGTTATTTT
CCACTGCAGTCCTACGGCTTCCAGCCCACCTATGGTGTCGGCTACCAGCCTTATCGCGTCGTGGTGCTGTCTTTT
GAGTTACTGCATGCACCTGCCACCGTGTGCGGCCCCAAGAAGTCCACCAACCTGGTCAAAAATAAATGCGTGAAT
TTCGGCAGCTCTGGAGGCTCTAGGGTGCAGCCAACTGAATCTATCGTGCGGTTTCCTAACATTACCAACCTGTGC
CCATTCGGCGAGGTCTTCAATGCTACCCGCTTCGCATCCGTGTACGCTTGGAACCGCAAGCGGATCAGTAACTGC
GTGGCCGACTACTCTGTGCTGTATAACTCTGCTTCCTTCAGCACCTTTAAATGTTACGGCGTGAGCCCAACTAAA
CTGAACGATCTGTGCTTTACGAACGTGTACGCCGACTCTTTTGTGATTCGCGGCGACGAAGTCAGGCAGATTGCT
CCTGGCCAGACAGGTAAAATTGCCGATTACAATTATAAGCTGCCAGATGATTTCACAGGTTGCGTGATCGCCTGG
AACTCTAATAACCTGGACTCAAAAGTCGGAGGTAATTATAACTACCTGTATCGCCTGTTCCGTAAGTCTAATCTG
AAGCCCTTTGAGAGGGACATCTCAACTGAAATTTACCAGGCCGGCAGCACACCTTGTAACGGTGTCGAAGGTTTT
AATTGCTATTTCCCTCTGCAGTCCTACGGTTTTCAGCCTACCTATGGCGTCGGCTACCAGCCATACCGGGTGGTG
GTGCTGTCCTTTGAGCTGCTGCATGCCCCCGCAACCGTGTGCGGCCCAAAGAAGAGCACCAACCTGGTCAAGAAC
AAATGCGTGAATTTTGGATCTGGCTCTGGCAGCAGAGTGCAGCCTACCGAGTCTATCGTGCGCTTTCCCAATATT
ACTAACCTGTGCCCTTTCGGCGAGGTCTTCAACGCTACCAGGTTTGCATCCGTGTACGCCTGGAATAGAAAGCGC
ATCTCTAACTGCGTGGCAGACTATAGCGTGCTGTACAATTCTGCTTCTTTTAGCACCTTTAAATGCTACGGCGTG
TCTCCTACTAAATTAAACGACCTGTGCTTCACTAACGTGTACGCAGATTCCTTTGTGATCAGAGGGGACGAAGTC
AGGCAGATCGCCCCAGGACAGACCGGAAAAATTGCCGACTACAACTACAAGCTGCCTGATGATTTCACAGGATGC
GTGATTGCATGGAACTCTAATAATCTGGATAGTAAAGTGGGGGTAATTACAACTATCTGTACAGACTGTTCCGCA
AATCTAATCTGAAACCATTTGAGAGAGATATTTCCACTGAAATTTATCAGGCCGGCAGCACCCCTTGCAACGGAG
TCGAAGGTTTTAACTGTTACTTCCCTCTGCAGAGCTACGGTTTTCAGCCAACCAATGGCGTGGGCTACCAGCCAT
ATCGCGTTGTGGTGCTGAGCTTTGAGCTACTGCATGCCCCCGCTACCGTGTGCGGCCCTAAGAAGTCTACCAACC
TGGTGAAAAATAAGTGCGTGAATTTTGGCTCTGGAGGAGGAGGATCCGGAGGAGGCGGCTCTGGCTCCGAGCCAA
AGTCTTGCGACAAGACCCATACATGCCCACCTTGTCCAGCTCCAGAGCTGCTGGGAGGACCATCCGTGTTCCTGT
TTCCACCCAAGCCCAAGGATACCCTGATGATCAGCAGGACCCCAGAGGTGACATGCGTGGTGGTGGACGTGTCTC
ACGAGGATCCCGAGGTGAAGTTTAACTGGTACGTGGACGGCGTGGAGGTGCATAATGCTAAGACCAAGCCTAGGG
AGGAGCAGTACAACAGCACCTATCGGGTGGTGTCTGTGCTGACAGTGCTGCACCAGGATTGGCTGAACGGCAAGG
AGTACAAGTGCGCCGTGTCCAATAAGGCCCTGCCTGCTCCAATCGAGAAGACCATCAGCAAGGCTAAGGGCCAGC
CCAGAGAGCCTCAGGTGTATACACTGCCTCCATCCCGCGACGAGCTGACCAAGAACCAAGTGAGCCTGACATGTC
TGGTGAAGGGCTTCTACCCCAGCGATATCGCCGTGGAGTGGGAGTCTAATGGCCAGCCTGAGAACAATTATAAGA
CCACACCCCGTGTGCTGGACTCCGATGGCAGCTTCTTTCTGTACTCTAAGCTGACCGTGGACAAGTCCCGGTGGC
AGCAGGGCAACGTGTTTAGCTGCTCTGTGATGCATGAGGCTCTGCACAATCATTACACACAGAAGTCCCTGAGCC
TGTCTCCTGGCAAGTGA
RBDs-Delta amino acid
(SEQ ID NO. 2)
MFVFLVLLPLVSSQCVRVQPTESIVRFPNITNLCPFGEVFNATRFASVYAWNRKRISNCVADYSVLYNSASFSTF
KCYGVSPTKLNDLCFTNVYADSFVIRGDEVRQJAPGQTGKIADYNYKLPDDFTGCVIAWNSNNLDSKVGGNYNYR
YRLFRKSNLKPFERDISTEIYQAGSKPCNGVEGFNCYFPLQSYGFQPTNGVGYQPYRVVVLSFELLHAPATVCGP
KKSTNLVKNKCVNFGSGSGSRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGENCYFPLQSYGFQPTNGVGYQPYRVVVLSFELLHAPAT
VCGPKKSTNLVKNKCVNFGSGSSGRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGTIADYNYKLPDDFTGCVIAWNSNNLDSK
VGGNYNYLYRLFRKSNLKPFERDISTEIYQAGSTPCNGVKGFNCYFPLQSYGFQPTYGVGYQPYRVVVLSFELLH
APATVCGPKKSTNLVKNKCVNFSGGSGSRVQPTESIVRFPNITNLCPFGEVENATRFASVYAWNRKRISNCVADY
SVLYNSASFSTFKCYGVSPTKLNDLCFTNVYADSFVIRGDEVRQIAPGQTGNIADYNYKLPDDFTGCVIAWNSNN
LDSKVGGNYNYLYRLFRKSNLKPFERDISTEIYQAGSTPCNGVKGFNCYFPLQSYGFQPTYGVGYQPYRVVVLSF
ELLHAPATVCGPKKSTNLVKNKCVNFGSSGGSRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGENCYFPLQSYGFQPTYGVGYQPYRVV
VLSFELLHAPATVCGPKKSTNLVKNKCVNFGSGSGSRVQPTESIVRFPNITNLCPFGEVENATRFASVYAWNRKR
ISNCVADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGENCYFPLQSYGFQPTNGVGYQP
YRVVVLSFELLHAPATVCGPKKSTNLVKNKCVNFGSGGGGSGGGGSGSEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK.
RBDs-Omicron DNA
(SEQ ID NO. 3)
ATGTTTGTGTTTCTAGTGCTACTACCACTAGTTTCCTCTCAGTGCGTGCGGGTGCAGCCCACAGAGTCCATCGTG
CGCTTCCCAAACATCACCAACCTGTGTCCATTCGGCGAAGTGTTCAATGCCACCAGATTTGCCTCTGTCTACGCC
TGGAACCGGAAAAGGATCTCTAACTGCGTGGCGGATTACTCCGTGCTGTACAACTCCGCCTCCTTCTCTACCTTC
AAGTGCTACGGCGTGAGCCCTACAAAGCTGAACGACCTGTGCTTTACAAACGTGTACGCTGATTCCTTCGTGATC
CGGGGCGACGAGGTCAGACAGATCGCACCTGGCCAAACCGGCAAGATCGCCGATTACAACTACAAGCTGCCTGAT
GACTTCACCGGTTGTGTGATCGCCTGGAACAGCAACAACCTGGACTCCAAGGTGGGCGGCAACTACAACTACAGA
TACAGACTGTTCAGAAAGTCCAACCTGAAGCTTTTCGAAAGAGACATCTCTACTGAAATCTACCAGGCCGGCTCT
AAGCCCTGCAACGGCGTGGAAGGCTTCAACTGTTACTTCCCTCTGCAGTCTTATGGCTTCCAACCTACCAACGGC
GTTGGCTACCAGCCTTACAGAGTGGTGGTGCTGTCCTTCGAGCTGCTACATGCCGCTGCTACCGTGTGTGGCCCT
AAGAAATCCACGAACCTGGTTAAGAACAAGTGGGTGAACTTTGGCTCCGGATCCGGTAGCCGGGTGCAGCCTACC
GAGTCCATTGTGCGGTTCCGCAACATCACCAACCTGTGTCCTTTCGACGAGGTGTTCAACGCTACCCGGTTTGCT
TCTGTGTACGCCTGGAACCGGAAGAGAATCAGCAATTGTGTGGCAGATTACTCCGTGCTGTACAATCTGGCCCCT
TTTTTCACCTTCAAGTGCTATGGCGTGAGCCCTACCAAGCTGAACGACTTGTGCTTTACAAACGTGTACGCCGAT
TCTTTCGTGATCAGAGGCGACGAGGTGAGACAAATAGCCCCCGGCCAGACCGGCAACATCGCAGATTACAACTAC
AAGCTACCTGACGACTTCACCGGCTGCGTGATCGCCTGGAACAGTAACAAGCTGGACTCCAAGGTGTCGGGAAAC
TACAACTACCTGTACCGCCTGTTCAGAAAGTCCAACCTGAAACCATTCGAGCGTGATATCTCTACCGAGATCTAC
CAGGCCGGCAACAAACCTTGTAACGGCGTGGCCGGATTTAACTGTTACTTTCCACTGAGATCCTACTCTTTCCGC
CCAACTTACGGTGTGGGCCACCAGCCTTACAGGGTCGTGGTGCTGTCCTTCGAGCTGCTGCACGCTCCTGCCACC
GTGTGCGGCCCCAAGAAATCTACAAATCTGGTGAAAAACAAGTGCGTGAATTTCGGATCTGGATCGTCTGGCAGA
GTGCAGCCTACAGAATCTATCGTGAGATTCCCTAACATCACCAACCTGTGCCCATTCGGCGAGGTGTTCAACGCC
ACACGGTTTGCTTCTGTTTACGCCTGGAACAGAAAGCGGATCTCTAACTGCGTGGCTGACTACTCGGTGCTGTAC
AACTCTGCTTCCTTCTCTACCTTCAAGTGCTACGGTGTAAGCCCTACAAAGCTGAACGACCTGTGCTTCACCAAT
GTGTACGCCGACTCCTTTGTGATCAGAGGGGATGAAGTGCGGCAGATCGCTCCTGGACAGACTGGCACCATCGCT
GACTACAACTACAAGCTGCCAGATGACTTCACGGGCTGCGTGATCGCCTGGAACTCCAACAACCTGGACAGCAAG
GTGGGGGGCAATTACAATTACCTGTACCGGCTGTTCCGCAAGAGTAACCTGAAGCCTTTCGAGAGAGACATCAGT
ACCGAGATCTACCAGGCTGGCAGTACCCCTTGCAACGGTGTGAAGGGCTTTAACTGTTACTTCCCTCTGCAGTCC
TACGGCTTCCAACCCACTTATGGAGTGGGCTACCAGCCCTATCGGGTGGTGGTACTGAGCTTCGAGCTGCTCCAT
GCTCCTGCCACCGTGTGCGGCCCAAAGAAGTCAACCAACCTGGTGAAGAACAAGTGTGTGAACTTCAGTGGCGGC
AGTGGCTGCCGGGTACAGCCTACAGAGAGCATCGTGCGTTTCCCGAACATCACCAACCTGTGCCCTTTCGGCGAG
GTTTTCAACGCCACCCGGTTCGCTTCTGTGTACGCCTGGAACCGGAAGAGAATCTCTAACTGCGTGGCCGATTAC
TCAGTCTTGTATAACTCCGCGAGCTTCTCTACCTTCAAGTGCTATGGCGTGTCCCCCACCAAGCTGAATGACTTG
TGCTTCACCAACGTGTACGCCGACTCCTTTGTGATCCGGGGCGATGAAGTGCGACAGATCGCCCCTGGCCAGACC
GGAAACATCGCCGACTACAACTATAAGCTGCCTGACGACTTCACCGGCTGTGTGATCGCCTGGAACTCCAACAAC
CTGGATTCGAAGGTGGGCGGCAACTACAACTATCTGTACAGACTGTTCAGAAAGTCTAACCTCAAGCCTTTTGAA
CGGGACATCTCCACCGAAATCTACCAGGCCGGCTCCACCCCCTGTAACGGCGTGAAGGGCTTCAACTGCTACTTC
CCTCTGCAGTCCTACGGCTTCCAGCCCACCTATGGCGTAGGCTATCAGCCCTACAGGGTGGTAGTGCTGTCCTTG
GAGCTGCTGCACGCTCCCGCAACCGTGTGCGGCCCTAAGAAGTCCACCAACCTGGTCAAGAATAAGTGCGTCAAC
TTCGGCTCCTCTGGCGGGAGCCGTGTGCAACCCACGGAATCGATTGTGCGGTTCCCTAACATCACCAACCTGTGC
CCTTTTGGTGAGGTTTTCAACGCCACCAGATTCGCTTCCGTGTACGCTTGGAACAGGAAGCGGATCTCCAACTGT
GTCGCCGACTACAGCGTGCTGTACAACTCTGCTTCCTTTTCTACCTTTAAGTGCTACGGAGTAAGCCCTACCAAG
CTGAACGACCTGTGTTTCACCAADGTGTACGCCGATTGTTTTGTGATCAGAGGAGACGAGGTTAGACAGATGGCT
CCTGGGCAGACCGGCAAGATCGCTGACTACAATTACAAGCTGCCCGACGACTTCACCGGATGCGTGATCGCTTGG
AATTCCAACAATCTGGACTCCAAGGTGGGCGGCAACTACAACTACCTGTACCGGCTGTTCAGAAAGTCCAATCTG
AAGCCCTTCGAGAGAGATATCTCCACAGAGATCTACCAAGCTGGCTCTACCCCTTGCAACGGCGTGGAAGGCTTC
AACTGCTACTTCCCCCTGCAGTGTTACGGCTTCCAGCCTACCTACGGCGTGGGCTACCAACCTTACAGAGTCGTG
GTCCTGAGCTTCGAGCTCCTGCACGCTCCTGCTACAGTGTGCGGACCTAAGAAGTCCACAAACCTCGTGAAGAAC
AAGTGCGTGÅACTTCGGGTCCGGCTCCGGCAGCCGGGTGCAGCCTACCGAGTCCATCGTCCGGTTCCCCAACATC
ACCAACCTGTGTCCTTTTGGCGAAGTGTTTAACGCCACCCGGTTCGCCTCCGTGTACGCTTGGAACCGGAAGAGA
ATCTCCAATTGCGTCGCGGACTACAGCGTCCTGTACAACTCCGCCTCTTTCTCGACCTTCAAATGCTACGGCGTG
TCCCCTACAAAACTGAATGACCTCTGCTTCACAAATGTGTACGCTGACTCTTTCGTGATTAGAGGCGACGAGGTA
CGGCAGATCGCCCCTGGCCAGACCGGCAAGATTGCCGACTACAACTACAAGCTGCCTGACGACTTCACCGGCTGC
GTGATCGCCTGGAACTCCAATAACCTGGACAGCAAGGTGGGAGGCAATTATAATTACCTGTATAGACTCTTCAGA
AAGTCTAACCTGAAGGCCTTCGAACGGGACATCTCCACAGAAATCTACCAGGCTGGTTCCACCCCCTGCAACGGC
GTGGAGGGCTTTAACTGCTACTTTCCTCTGCAATCCTACGGCTTTCAGCCTACCAACGGAGTGGGCTACCAGCCT
TATAGAGTGGTTGTGCTGTCCTTTGAGCTGCTGCACGCCCCTGCCACAGTGTGCGGCCCCAAGAAATCTACCAAC
CTGGTAAAAAACAAATGCGTGAACTTCGGCTCCGGGGGGGGAGGGTCAGGCGGCGGCGGCTCCGGCTCCGAGCCT
AAATCCTGCGATAAGACCCACACCTGTCCACCTTGCCCTGCCCCTGAACTGCTGGGCGGGCCTTCTGTGTTCCTG
TTCCCCCCTAAGCCTAAGGACACCCTGATGATCTCTAGAACCCCTGAGGTGACCTGCGTGGTAGTGGACGTGTCT
CATGAGGACCCCGAAGTCAAGTTCAACTGGTACGTGGATGGCGTGGAAGTGCACAACGCCAAAACCAAACCTAGA
GAGGAACAGTACAACTCCACCTACCGGGTGGTGTCCGTGCTGACCGTGCTCCACCAGGACTGGCTGAACGGAAAA
GAGTACAAGTGCGCCGTGTCTAATAAGGCTCTGCCAGCTCCTATCGAGAAGACCATCTCCAAGGCCAAGGGCCAG
CCTAGAGAACCTCAGGTCTACACCCTGCCTCCATCTAGAGATGAACTGACCAAGAACCAGGTGTCCCTGACCTGC
CTGGTGAAGGGCTTCTACCCTTCTGACATCGCTGTGGAATGGGAGTCCAACGGACAGCCTGAGAACAACTACAAG
ACCACGCCTCCTGTGCTGGATTGCGACGGCAGCTTCTTCCTGTACTCCAAGCTGACCGTGGACAAATCCAGATGG
CAGCAGGGCAATGTGTTCTCCTGCTCCGTGATGCACGAGGCCCTGCACAACCACTACACCCAGAAGAGCCTGTCT
CTGTCTCCTGGTAAATAA
RBDs-Omicron amino acid
(SEQ TD NO. 4)
MFVFLVLLPLVSSQCVRVQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVIAWNSNNLDSKVGGNYNYR
YRLERKSNLKPFERDISTEIYQAGSKPCNGVEGENCYFPLQSYGFQPTNGVGYQPYRVVVLSFELLHAPATVCGP
KKSTNLVKNKCVNFGSGSGSRVQPTESIVRFPNITNLCPFDEVFNATRFASVYAWNRKRISNCVADYSVLYNLAP
FFTFKCYGVSPTKLNDLCFTNVYADSFVIRGDEVRQIAPGQTGNIADYNYKLPDDFTGCVIAWNSNKLDSKVSGN
YNYLYRLFRKSNLKPFERDISTEIYQAGNKPCNGVAGENCYFPLRSYSFRPTYGVGHQPYRVVVLSFELLHAPAT
VCGPKKSTNLVKNKCVNFGSGSSGRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGTIADYNYKLPDDFTGCVIAWNSNNLDSK
VGGNYNYLYRLFRKSNLKPFERDISTEIYQAGSTPCNGVKGFNCYFPLQSYGFQPTYGVGYQPYRVVVLSFELLH
APATVCGPKKSTNLVKNKCVNFSGGSGSRVQPTESIVRFPNITNLCPFGEVENATRFASVYAWNRKRISNCVADY
SVLYNSASFSTFKCYGVSPTKLNDLCFTNVYADSFVIRGDEVRQIAPGQTGNIADYNYKLPDDFTGCVIAWNSNN
LDSKVGGNYNYLYRLFRKSNLKPFERDISTEIYQAGSTPCNGVKGFNCYFPLQSYGFQPTYGVGYQPYRVVVLSF
ELLHAPATVCGPKKSTNLVKNKCVNFGSSGGSRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGENCYFPLQSYGFQPTYGVGYQPYRVV
VLSFELLHAPATVCGPKKSTNLVKNKCVNFGSGSGSRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGENCYFPLQSYGFQPTNGVGYQP
YRVVVLSFELLHAPATVCGPKKSTNLVKNKCVNFGSGGGGSGGGGSGSEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTCLVKGFYPSDIAVEWESNGQPENNYK
TTPPVLQSDGSFFLYSKLTVDKSRWQQGNVFSCSVMHEALHNHYTQKSLSLSPGK.
SARS-CoV-2 RBDs cDNA
(FIG. 8B; (SEQ ID NO. 5)
ATGTTCGTGTTTCTGGTGCTGCTGCCTCTGGTGTCCAGCCAGTGCGTGAGGGTGCAGCCAACCGAGAGCATCGTG
CGGTTCCCAAACATCACAAATCTGTGCCCCTTCGGCGAGGTGTTTAACGCCACCCGGTTTGCTTCCGTGTACGCC
TGGAACAGGAAGCGGATCTCCAATTGCGTGGCCGACTACAGCGTGCTGTATAATTCCGCTAGCTTCTCTACATTT
AAGTGCTATGGCGTGTCTCCCACCAAGCTGAACGATCTGTGCTTCACAAACGTGTACGCCGACTCCTTTGTGATC
AGGGGAGATGAAGTGAGGCAGATCGCTCCAGGACAGACCGGCAAGATCGCTGACTACAACTATAAGCTGCCCGAC
GATTTCACAGGCTGCGTGATCGCTTGGAATTCTAACAATCTGGATTCCAAAGTGGGCGGCAACTACAATTATAGA
TACCGCCTGTTCAGAAAGAGCAACCTGAAGCCTTTTGAGCGCGACATCAGCACCGAGATCTATCAGGCCGGCTCT
AAGCCATGCAACGGCGTGGAGGGCTTCAATTGTTACTTTCCACTGCAGTCTTATGGCTTCCAGCCCACAAACGGC
GTGGGCTACCAGCCTTATAGGGTGGTGGTGCTGAGCTTTGAGCTGCTGCACGCTCCAGCTACCGTGTGCGGACCC
AAGAAGTCTACAAACCTGGTGAAGAATAAATGCGTGAACTTCGGATCCGGAAGCGGCTCTAGGGTGCAGCCTACC
GAGTCCATCGTGCGGTTTCCTAATATTACAAATCTGTGCCCATTCGGCGAAGTCTTTAACGCTACCCGGTTCGCC
AGCGTGTACGCTTGGAATAGAAAGCGGATCTCTAACTGCGTGGCTGACTACTCCGTGCTGTACAACTCCGCTTCT
TTTTCTACATTTAAATGTTATGGCGTGTCCCCAACTAAACTGAATGACCTGTGCTTTACCAACGTGTACGCCGAC
AGCTTTGTGATCAGAGGCGACGAAGTCAGACAGATCGCTCCTGGCCAGACTGGCAAGATCGCTGACTATAACTAC
AAGCTGCCAGACGACTTCACCGGTTGCGTGATTGCTTGGAATAGCAACAATCTGGATTCTAAAGTGGGCGGCAAC
TATAATTACAGATATCGCCTGTTCAGAAAGTCCAACCTGAAGCCCTTTGAGCGCGATATCTCTACTGAGATCTAT
CAGGCTGGCAGCACACCTTGTAATGGTGTCGAAGGTTTTAACTGCTATTTTCCACTGCAGAGCTATGGCTTCCAG
CCAACCAACGGAGTCGGCTACCAGCCATATAGAGTGGTGGTGCTGTCTTTTGAGCTGCTGCATGCTCCTGCTACC
GTGTGCGGACCAAAGAAGTCCACAAATCTGGTGAAGAACAAGTGCGTGAATTTCGGATCTGGCTCTTCCGGAAGA
GTCCAGCCAACTGAATCCATCGTGCGGTTTCCCAATATTACCAATCTGTGCCCTTTCGGCGAAGTCTTCAACGCC
ACCCGCTTCGCCTCCGTGTACGCTTGGAACAGAAAACGGATCTCTAATTGCGTGGCTGACTACTCTGTGCTGTAC
AACTCCGCTAGTTTTTCTACATTTAAGTGTTACGGCGTGAGCCCTACTAAACTGAATGATCTGTGCTTTACTAAC
GTGTACGCTGACTCTTTTGTGATTAGAGGCGACGAAGTCCGCCAGATTGCCCCAGGACAGACCGGCACAATTGCC
GATTATAACTATAAGCTGCCTGACGACTTCACCGGATGCGTGATTGCCTGGAACTCCAACAATCTGGATAGTAAA
GTCGGTGGCAACTACAATTATCTGTACAGGCTGTTCCGCAAGTCCAACCTGAAGCCATTTGAGAGGGACATCTCT
ACCGAGATCTACCAGGCTGGATCCACACCATGCAACGGAGTGAAAGGTTTTAACTGCTACTTCCCACTGCAGTCA
TACGGCTTCCAGCCAACCTACGGAGTGGGCTACCAGCCATACAGAGTCGTGGTGCTGAGCTTTGAGTTACTGCAT
GCCCCCGCCACCGTGTGCGGACCTAAGAAGAGCACCAATCTGGTCAAAAACAAATGCGTGAACTTCTCTGGAGGA
TCCGGCAGCAGAGTGCAGCCAACCGAGTCCATCGTGCGCTTTCCTAATATCACCAACCTGTGCCCCTTCGGCGAA
GTGTTTAACGCTACCAGATTTGCTAGCGTGTACGCATGGAATAGAAAGCGCATTTCTAACTGCGTGGCAGATTAT
AGCGTGCTGTATAATTCTGCTTCCTTCAGCACATTTAAATGTTACGGCGTGTCCCCTACTAAACTGAACGACCTG
TGCTTTACAAACGTGTACGCTGATAGCTTTGTGATCAGAGGTGACGAAGTCCGCCAGATCGCCCCTGGCCAGACC
GGCAACATCGCTGACTATAATTACAAGCTGCCCGACGACTTCACCGGGTGCGTGATTGCTTGGAACAGCAATAAC
CTGGACAGCAAAGTCGGTGGTAACTATAATTACCTGTATAGACTGTTCCGCAAGTCTAATCTGAAGCCTTTTGAG
AGAGATATCAGCACCGAGATCTACCAGGCCGGCTCTACACCATGCAACGGCGTGAAGGGCTTCAATTGTTATTTT
CCACTGCAGTCCTACGGCTTCCAGCCCACCTATGGTGTCGGCTACCAGCCTTATCGCGTCGTGGTGCTGTCTTTT
GAGTTACTGCATGCACCTGCCACCGTGTGCGGCCCCAAGAAGTCCACCAACCTGGTCAAAAATAAATGCGTGAAT
TTCGGCAGCTCTGGAGGCTCTAGGGTGCAGCCAACTGAATCTATCGTGCGGTTTCCTAACATTACCAACCTGTGC
CCATTCGGCGAGGTCTTCAATGCTACCCGCTTCGCATCCGTGTACGCTTGGAACCGCAAGCGGATCAGTAACTGC
GTGGCCGACTACTCTGTGCTGTATAACTCTGCTTCCTTCAGCACCTTTAAATGTTACGGCGTGAGCCCAACTAAA
CTGAACGATCTGTGCTTTACGAACGTGTACGCCGACTCTTTTGTGATTCGCGGCGACGAAGTCAGGCAGATTGCT
CCTGGCCAGACAGGTAAAATTGCCGATTACAATTATAAGCTGCCAGATGATTTCACAGGTTGCGTGATCGCCTGG
AACTCTAATAACCTGGACTCAAAAGTCGGAGGTAATTATAACTACCTGTATCGCCTGTTCCGTAAGTCTAATCTG
AAGCCCTTTGAGAGGGACATCTCAACTGAAATTTACCAGGCCGGCAGCACACCTTGTAACGGTGTCGAAGGTTTT
AATTGCTATTTCCCTCTGCAGTCCTACGGTTTTCAGCCTACCTATGGCGTCGGCTACCAGCCATACCGGGTGGTG
GTGCTGTCCTTTGAGCTGCTGCATGCCCCCGCAACCGTGTGCGGCCCAAAGAAGAGCACCAACCTGGTCAAGAAC
AAATGCGTGAATTTTGGATCTGGCTCTGGCAGCAGAGTGCAGCCTACCGAGTCTATCGTGCGCTTTCCCAATATT
ACTAACCTGTGCCCTTTCGGCGAGGTCTTCAACGCTACCAGGTTTGCATCCGTGTACGCCTGGAATAGAAAGCGC
ATCTCTAACTGCGTGGCAGACTATAGCGTGCTGTACAATTCTGCTTCTTTTAGCACCTTTAAATGCTACGGCGTG
TCTCCTACTAAATTAAACGACCTGTGCTTCACTAACGTGTACGCAGATTCCTTTGTGATCAGAGGGGACGAAGTC
AGGCAGATCGCCCCAGGACAGACCGGAAAAATTGCCGACTACAACTACAAGCTGCCTGATGATTTCACAGGATGC
GTGATTGCATGGAACTCTAATAATCTGGATAGTAAAGTGGGTGGTAATTACAACTATCTGTACAGACTGTTCCGC
AAATCTAATCTGAAACCATTTGAGAGAGATATTTCCACTGAAATTTATCAGGCCGGCAGCACCCCTTGCAACGGA
GTCGAAGGTTTTAACTGTTACTTCCCTCTGCAGAGCTACGGTTTTCAGCCAACCAATGGCGTGGGCTACCAGCCA
TATCGCGTTGTGGTGCTGAGCTTTGAGCTACTGCATGCCCCCGCTACCGTGTGCGGCCCTAAGAAGTCTACCAAC
CTGGTGAAAAATAAGTGCGTGAATTTTCATCACCACCATCACCACCATCACTGA
SARS-CoV-2 RBDs Amino Acid
(FIG. 8B; (SEQ ID NO. 6)
MFVFLVLLPLVSSQCVRVQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVIAWNSNNLDSKVGGNYNYR
YRLFRKSNLKPFERDISTEIYQAGSKPCNGVEGFNCYFPLQSYGFQPTNGVGYQPYRVVVLSFELLHAPATVCGP
KKSTNLVKNKCVNFGSGSGSRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGENCYFPLQSYGFQPTNGVGYQPYRVVVLSFELLHAPAT
VCGPKKSTNLVKNKCVNFGSGSSGRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGTIADYNYKLPDDFTGCVIAWNSNNLDSK
VGGNYNYLYRLFRKSNLKPFERDISTEIYQAGSTPCNGVKGFNCYFPLQSYGFQPTYGVGYQPYRVVVLSFELLH
APATVCGPKKSTNLVKNKCVNFSGGSGSRVQPTESIVRFPNITNLCPFGEVENATRFASVYAWNRKRISNCVADY
SVLYNSASFSTFKCYGVSPTKLNDLCFTNVYADSFVIRGDEVRQIAPGQTGNIADYNYKLPDDFTGCVIAWNSNN
LDSKVGGNYNYLYRLFRKSNLKPFERDISTEIYQAGSTPCNGVKGFNCYFPLQSYGFQPTYGVGYQPYRVVVLSF
ELLHAPATVCGPKKSTNLVKNKCVNFGSSGGSRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGENCYFPLQSYGFQPTYGVGYQPYRVV
VLSFELLHAPATVCGPKKSTNLVKNKCVNFGSGSGSRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGENCYFPLQSYGFQPTNGVGYQP
YRVVVLSFELLHAPATVCGPKKSTNLVKNKCVNFHHHHHHHH
SARS-CoV-2 RBDs-Fc cDNA
(FIG. 8C; (SEQ ID NO. 7)
ATGTTCGTGTTTCTGGTGCTGCTGCCTCTGGTGTCCAGCCAGTGCGTGAGGGTGCAGCCAACCGAGAGCATCGTG
CGGTTCCCAAACATCACAAATCTGTGCCCCTTCGGCGAGGTGTTTAACGCCACCCGGTTTGCTTCCGTGTACGCC
TGGAACAGGAAGCGGATCTCCAATTGCGTGGCCGACTACAGCGTGCTGTATAATTCCGCTAGCTTCTCTACATTT
AAGTGCTATGGCGTGTCTCCCACCAAGCTGAACGATCTGTGCTTCACAAACGTGTACGCCGACTCCTTTGTGATC
AGGGGAGATGAAGTGAGGCAGATCGCTCCAGGACAGACCGGCAAGATCGCTGACTACAACTATAAGCTGCCCGAC
GATTTCACAGGCTGCGTGATCGCTTGGAATTCTAACAATCTGGATTCCAAAGTGGGCGGCAACTACAATTATAGA
TACCGCCTGTTCAGAAAGAGCAACCTGAAGCCTTTTGAGCGCGACATCAGCACCGAGATCTATCAGGCCGGCTCT
AAGCCATGCAACGGCGTGGAGGGCTTCAATTGTTACTTTCCACTGCAGTCTTATGGCTTCCAGCCCACAAACGGC
GTGGGCTACCAGCCTTATAGGGTGGTGGTGCTGAGCTTTGAGCTGCTGCACGCTCCAGCTACCGTGTGCGGACCC
AAGAAGTCTACAAACCTGGTGAAGAATAAATGCGTGAACTTCGGATCCGGAAGCGGCTCTAGGGTGCAGCCTACC
GAGTCCATCGTGCGGTTTCCTAATATTACAAATCTGTGCCCATTCGGCGAAGTCTTTAACGCTACCCGGTTCGCC
AGCGTGTACGCTTGGAATAGAAAGCGGATCTCTAACTGCGTGGCTGACTACTCCGTGCTGTACAACTCCGCTTCT
TTTTCTACATTTAAATGTTATGGCGTGTCCCCAACTAAACTGAATGACCTGTGCTTTACCAACGTGTACGCCGAC
AGCTTTGTGATCAGAGGCGACGAAGTCAGACAGATCGCTCCTGGCCAGACTGGCAAGATCGCTGACTATAACTAC
AAGCTGCCAGACGACTTCACCGGTTGCGTGATTGCTTGGAATAGCAACAATCTGGATTCTAAAGTGGGCGGCAAC
TATAATTACAGATATCGCCTGTTCAGAAAGTCCAACCTGAAGCCCTTTGAGCGCGATATCTCTACTGAGATCTAT
CAGGCTGGCAGCACACCTTGTAATGGTGTCGAAGGTTTTAACTGCTATTTTCCACTGCAGAGCTATGGCTTCCAG
CCAACCAACGGAGTCGGCTACCAGCCATATAGAGTGGTGGTGCTGTCTTTTGAGCTGCTGCATGCTCCTGCTACC
GTGTGCGGACCAAAGAAGTCCACAAATCTGGTGAAGAACAAGTGCGTGAATTTCGGATCTGGCTCTTCCGGAAGA
GTCCAGCCAACTGAATCCATCGTGCGGTTTCCCAATATTACCAATCTGTGCCCTTTCGGCGAAGTCTTCAACGCC
ACCCGCTTCGCCTCCGTGTACGCTTGGAACAGAAAACGGATCTCTAATTGCGTGGCTGACTACTCTGTGCTGTAC
AACTCCGCTAGTTTTTCTACATTTAAGTGTTACGGCGTGAGCCCTACTAAACTGAATGATCTGTGCTTTACTAAC
GTGTACGCTGACTCTTTTGTGATTAGAGGCGACGAAGTCCGCCAGATTGCCCCAGGACAGACCGGCACAATTGCC
GATTATAACTATAAGCTGCCTGACGACTTCACCGGATGCGTGATTGCCTGGAACTCCAACAATCTGGATAGTAAA
GTCGGTGGCAACTACAATTATCTGTACAGGCTGTTCCGCAAGTCCAACCTGAAGCCATTTGAGAGGGACATCTCT
ACCGAGATCTACCAGGCTGGATCCACACCATGCAACGGAGTGAAAGGTTTTAACTGCTACTTCCCACTGCAGTCA
TACGGCTTCCAGCCAACCTACGGAGTGGGCTACCAGCCATACAGAGTCGTGGTGCTGAGCTTTGAGTTACTGCAT
GCCCCCGCCACCGTGTGCGGACCTAAGAAGAGCACCAATCTGGTCAAAAACAAATGCGTGAACTTCTCTGGAGGA
TCCGGCAGCAGAGTGCAGCCAACCGAGTCCATCGTGCGCTTTCCTAATATCACCAACCTGTGCCCCTTCGGCGAA
GTGTTTAACGCTACCAGATTTGCTAGCGTGTACGCATGGAATAGAAAGCGCATTTCTAACTGCGTGGCAGATTAT
AGCGTGCTGTATAATTCTGCTTCCTTCAGCACATTTAAATGTTACGGCGTGTCCCCTACTAAACTGAACGACCTG
TGCTTTACAAACGTGTACGCTGATAGCTTTGTGATCAGAGGTGACGAAGTCCGCCAGATCGCCCCTGGCCAGACC
GGCAACATCGCTGACTATAATTACAAGCTGCCCGACGACTTCACCGGGTGCGTGATTGCTTGGAACAGCAATAAC
CTGGACAGCAAAGTCGGTGGTAACTATAATTACCTGTATAGACTGTTCCGCAAGTCTAATCTGAAGCCTTTTGAG
AGAGATATCAGCACCGAGATCTACCAGGCCGGCTCTACACCATGCAACGGCGTGAAGGGCTTCAATTGTTATTTT
CCACTGCAGTCCTACGGCTTCCAGCCCACCTATGGTGTCGGCTACCAGCCTTATCGCGTCGTGGTGCTGTCTTTT
GAGTTACTGCATGCACCTGCCACCGTGTGCGGCCCCAAGAAGTCCACCAACCTGGTCAAAAATAAATGCGTGAAT
TTCGGCAGCTCTGGAGGCTCTAGGGTGCAGCCAACTGAATCTATCGTGCGGTTTCCTAACATTACCAACCTGTGC
CCATTCGGCGAGGTCTTCAATGCTACCCGCTTCGCATCCGTGTACGCTTGGAACCGCAAGCGGATCAGTAACTGC
GTGGCCGACTACTCTGTGCTGTATAACTCTGCTTCCTTCAGCACCTTTAAATGTTACGGCGTGAGCCCAACTAAA
CTGAACGATCTGTGCTTTACGAACGTGTACGCCGACTCTTTTGTGATTCGCGGCGACGAAGTCAGGCAGATTGCT
CCTGGCCAGACAGGTAAAATTGCCGATTACAATTATAAGCTGCCAGATGATTTCACAGGTTGCGTGATCGCCTGG
AACTCTAATAACCTGGACTCAAAAGTCGGAGGTAATTATAACTACCTGTATCGCCTGTTCCGTAAGTCTAATCTG
AAGCCCTTTGAGAGGGACATCTCAACTGAAATTTACCAGGCCGGCAGCACACCTTGTAACGGTGTCGAAGGTTTT
AATTGCTATTTCCCTCTGCAGTCCTACGGTTTTCAGCCTACCTATGGCGTCGGCTACCAGCCATACCGGGTGGTG
GTGCTGTCCTTTGAGCTGCTGCATGCCCCCGCAACCGTGTGCGGCCCAAAGAAGAGCACCAACCTGGTCAAGAAC
AAATGCGTGAATTTTGGATCTGGCTCTGGCAGCAGAGTGCAGCCTACCGAGTCTATCGTGCGCTTTCCCAATATT
ACTAACCTGTGCCCTTTCGGCGAGGTCTTCAACGCTACCAGGTTTGCATCCGTGTACGCCTGGAATAGAAAGCGC
ATCTCTAACTGCGTGGCAGACTATAGCGTGCTGTACAATTCTGCTTCTTTTAGCACCTTTAAATGCTACGGCGTG
TCTCCTACTAAATTAAACGACCTGTGCTTCACTAACGTGTACGCAGATTCCTTTGTGATCAGAGGGGACGAAGTC
AGGCAGATCGCCCCAGGACAGACCGGAAAAATTGCCGACTACAACTACAAGCTGCCTGATGATTTCACAGGATGC
GTGATTGCATGGAACTCTAATAATCTGGATAGTAAAGTGGGTGGTAATTACAACTATCTGTACAGACTGTTCCGC
AAATCTAATCTGAAACCATTTGAGAGAGATATTTCCACTGAAATTTATCAGGCCGGCAGCACCCCTTGCAACGGA
GTCGAAGGTTTTAACTGTTACTTCCCTCTGCAGAGCTACGGTTTTCAGCCAACCAATGGCGTGGGCTACCAGCCA
TATCGCGTTGTGGTGCTGAGCTTTGAGCTACTGCATGCCCCCGCTACCGTGTGCGGCCCTAAGAAGTCTACCAAC
CTGGTGAAAAATAAGTGCGTGAATTTTGGCTCTGGAGGAGGAGGATCCGGAGGAGGCGGCTCTGGCTCCGAGCCA
AAGTCTTGCGACAAGACCCATACATGCCCACCTTGTCCAGCTCCAGAGCTGCTGGGAGGACCATCCGTGTTCCTG
TTTCCACCCAAGCCCAAGGATACCCTGATGATCAGCAGGACCCCAGAGGTGACATGCGTGGTGGTGGACGTGTCT
CACGAGGATCCCGAGGTGAAGTTTAACTGGTACGTGGACGGCGTGGAGGTGCATAATGCTAAGACCAAGCCTAGG
GAGGAGCAGTACAACAGCACCTATCGGGTGGTGTCTGTGCTGACAGTGCTGCACCAGGATTGGCTGAACGGCAAG
GAGTACAAGTGCGCCGTGTCCAATAAGGCCCTGCCTGCTCCAATCGAGAAGACCATCAGCAAGGCTAAGGGCCAG
CCCAGAGAGCCTCAGGTGTATACACTGCCTCCATCCCGCGACGAGCTGACCAAGAACCAAGTGAGCCTGACATGT
CTGGTGAAGGGCTTCTACCCCAGCGATATCGCCGTGGAGTGGGAGTCTAATGGCCAGCCTGAGAACAATTATAAG
ACCACACCCCCTGTGCTGGACTCCGATGGCAGCTTCTTTCTGTACTCTAAGCTGACCGTGGACAAGTCCCGGTGG
CAGCAGGGCAACGTGTTTAGCTGCTCTGTGATGCATGAGGCTCTGCACAATCATTACACACAGAAGTCCCTGAGC
CTGTCTCCTGGCAAGTGA
SARS-CoV-2 RBDs-Fc amino acid
(FIG. 8C; (SEQ ID NO. 8)
MFVFLVLLPLVSSQCVRVQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVIAWNSNNLDSKVGGNYNYR
YRLFRKSNLKPFERDISTEIYQAGSKPCNGVEGENCYFPLQSYGFQPTNGVGYQPYRVVVLSFELLHAPATVCGP
KKSTNLVKNKCVNFGSGSGSRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVGGN
YNYRYRLFRKSNLKPFERDISTEIYQAGSTPCNGVEGFNCYFPLQSYGFQPTNGVGYQPYRVVVLSFELLHAPAT
VCGPKKSTNLVKNKCVNFGSGSSGRVQPTESIVRFPNITNLCPFGEVENATRFASVYAWNRKRISNCVADYSVLY
NSASFSTFKCYGVSPTKLNDLCFTNVYADSFVIRGDEVRQIAPGQTGTIADYNYKLPDDFTGCVIAWNSNNLDSK
VGGNYNYLYRLFRKSNLKPFERDISTEIYQAGSTPCNGVKGFNCYFPLQSYGFQPTYGVGYQPYRVVVLSFELLH
APATVCGPKKSTNLVKNKCVNFSGGSGSRVQPTESIVRFPNITNLCPFGEVFNATRFASVYAWNRKRISNCVADY
SVLYNSASFSTFKCYGVSPTKLNDLCFTNVYADSFVIRGDEVRQIAPGQTGNIADYNYKLPDDFTGCVIAWNSNN
LDSKVGGNYNYLYRLFRKSNLKPFERDISTEIYQAGSTPCNGVKGFNCYFPLQSYGFQPTYGVGYQPYRVVVLSF
ELLHAPATVCGPKKSTNLVKNKCVNFGSSGGSRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGCVIAW
NSNNLDSKVGGNYNYLYRLFRKSNLKPFERDISTEIYQAGSTPCNGVEGFNCYFPLQSYGFQPTYGVGYQPYRVV
VLSFELLHAPATVCGPKKSTNLVKNKCVNFGSGSGSRVQPTESIVRFPNITNLCPFGEVFNATRFASVYAWNRKR
ISNCVADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPLQSYGFQPTNGVGYQP
YRVVVLSFELLHAPATVCGPKKSTNLVKNKCVNFGSGGGGSGGGGSGSEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK.
SARS-CoV-2 Omicron subvariants RBDs cDNA
(FIG. 8D; (SEQ ID NO. 9)
ATGTTTGTGTTTCTAGTGCTACTACCACTAGTGAGCAGCCAGTGCGTTAGAGTGCAGCCTACCGAAAGTATCGTG
AGATTTCCCAACATCACCAACCTGTGCCCATTCGACGAAGTGTTCAACGCTACAAAGTTCGCCTCTGTATACGCC
TGGAACAGAAAGCGGATCAGCAACTGCGTTGCCGACTACTCTGTTCTGTACAATCTGGCCCCTTTCTTCACCTTC
AAGTGTTACGGCGTTTCCCCCACCAAACTGAACGACCTGTGCTTCACTAACGTGTACGCTGATTCCTTCGTGATC
CGAGGCGACGAGGTGAGACAGATCGCCCCTGGCCAGACCGGAAACATAGCCGACTACAACTATAAGCTGCCGGAC
GACTTCACCGGCTGCGTGATCGCTTGGAATTCCAACAAATTGGACTCCAAGGTGTCCGGTAACTACAACTACCTG
TACAGACTGTTTAGAAAGTCCAATCTGAAACCCTTCGAAAGAGACATCTCCACCGAGATCTACCAGGCAGGCAAC
AAACCCTGCAACGGAGTGGCAGGCTTCAACTGCTACTTTCCTCTGCGTTCCTACTCTTTCAGACCAACCTACGGC
GTGGGCCACCAACCTTACCGCGTGGTGGTGCTGAGTTTCGAGCTGCTGCACGCTCCTGCCACAGTGTGCGGCCCT
AAGAAGTCTACAAACCTGGTCAAGAACAAGTGCGTGAATTTCGGCTCCGGCTCTGGCTCTAGAGTGCAGCCCACC
GAGTCTATCGTGAGATTCCCAAACATCACCAACTTGTGTCCTTTCGATGAGGTGTTCAACGCCACCAGATTCGCC
TCCGTGTACGCGTGGAACAGAAAGCGCATCAGTAATTGTGTGGCCGACTACTCCGTGCTGTACAACTTCGCCCCC
TTCTTCGCTTTCAAGTGCTACGGAGTATCTCCCACCAAACTGAACGACCTGTGCTTCACCAACGTGTACGCCGAC
TCCTTCGTGATCCGGGGAAACGAGGTTAGCCAGATTGCTCCGGGACAGACAGGGAACATCGCAGATTACAACTAC
AAGCTGCCCGACGACTTCACCGGCTGCGTGATCGCGTGGAACTCCAATAAGCTCGACTCCAAGGTCGGAGGCAAC
TATAACTACCTGTACAGACTGTTCCGGAAGTCCAATCTGAAGCCCTTCGAGAGAGACATCTCTACAGAGATCTAC
CAAGCTGGCAACAAGCCTTGCAACGGCGTGGCTGGTTTCAACTGCTACTTCCCCCTGCGGTCATACGGCTTCAGA
CCCACCTATGGTGTGGGCCACCAGCCTTATAGAGTGGTGGTGCTGAGCTTCGAGTTGCTGCATGCCCCTGCCACC
GTGTGTGGCCCCAAGAAGTCTACCAATCTGGTGAAGAACAAGTGTGTGAACTTCGGCTCAGGCAGCTCCGGCAGA
GTGCAGCCCACCGAGTCTATCGTGCGGTTCCCCAACATCACTAACCTGTGCCCCTTCCACGAGGTGTTCAACGCC
ACCACCTTCGCCTCCGTGTACGCCTGGAACCGGAAGAGAATCTCGAACTGCGTGGCCGACTACAGCGTCCTGTAC
AATTTTGCCCCTTTTTTTGCCTTCAAATGCTACGGCGTGTCCCCTACCAAGCTGAACGATCTGTGTTTCACAAAC
GTGTACGCTGATTCGTTCGTGATCCGGGGCAACGAAGTGTCGCAAATCGCACCTGGACAGACCGGCAACATCGCA
GACTACAACTACAAGCTGCCTGACGACTTCACGGGCTGTGTAATCGCCTGGAACTCCAACAAGCTGGATTCCAAG
GTGTCCGGCAATTACAACTACCTGTACCGGTTGTTCCGGAAGTCCAAGCTGAAGCCTTTCGAGAGAGATATCAGC
ACAGAGATCTATCAGGCCGGCAACAAGCCTTGTAATGGCGTCGCTGGCTCTAACTGCTACTTCCCTCTGCAGTCC
TACGGTTTCCGGCCAACCTACGGCGTGGGCCACCAGCCTTACAGAGTGGTCGTGTTGTCTTTCGAACTGCTGCAC
GCTCCTGCTACCGTGTGCGGCCCTAAGAAGTCCACCAACCTGGTGAAGAATAAGTGCGTCAATTTCTCCGGCGGT
TCCGGCTCTAGAGTGCAGCCTACAGAGTCCATCGTGAGATTCCCTAATATCACCAATCTGTGCCCTTTCGACGAG
GTGTTCAACGCTACCACATTCGCCTCTGTGTATGCTTGGAATAGAAAGCGGATCAGCAACTGCGTGGCCGACTAC
TCCGTGCTGTACAACTTCGCTCCTTTCTTCGCCTTCAAGTGCTACGGCGTGTCCCCTACCAAACTGAACGATCTG
TGCTTCACCAATGTGTACGCTGATTCTTTCGTGATAAGAGGCAACGAGGTCTCCCAGATCGCCCCTGGCCAGACC
GGCAACATAGCCGATTATAACTACAAGCTGCCCGACGATTTTACCGGCTGTGTCATCGCCTGGAACTCCAACAAG
CTGGATAGTACCGTGGGAGGCAACTACAACTACAGATACCGGCTGTTCAGAAAGTCTAAGCTGAAGCCATTCGAA
CGGGACATCTCTACCGAAATCTACCAGGCTGGCAACAAGCCTTGCAACGGCGTGGCCGGAGTCAACTGCTACTTC
CCTCTGCAGTCTTACGGATTCCGGCCTACTTACGGCGTGGGCCATCAGCCCTACAGAGTGGTTGTCCTGTCCTTT
GAACTGCTGCACGCCCCTGCGACCGTGTGCGGACCTAAGAAATCCACCAATCTCGTCAAAAACAAGTGCGTGAAC
TTTGGCAGCTCCGGCGGCTCTCGGGTGCAGCCTACCGAGTCCATCGTGCGGTTCCCTAACATCACAAACCTGTGT
CCTTTCCACGAGGTGTTCAATGCCACCACCTTTGCTTCTGTGTACGCATGGAACAGAAAGCGGATCTCCAACTGT
GTGGCCGACTACTCCGTGATCTACAACTTCGCCCCCTTCTTCGCTTTCAAGTGCTACGGGGTATCCCCCACAAAG
CTGAACGACCTGTGTTTTACCAATGTCTACGCTGACTCTTTTGTGATCCGGGGCAACGAGGTGTCTCAGATCGCT
CCTGGCCAGACCGGCAATATTGCCGACTACAACTACAAACTGCCTGACGACTTTACCGGATGTGTGATCGCCTGG
AACTCTAACAAACTGGACTCCAAACCTTCTGGCAACTACAACTACCTGTATCGGCTGTTCAGAAAGTCCAAGTTG
AAACCTTTCGAGCGGGACATCTCCACCGAAATCTACCAGGCTGGCAACAAACCTTGTAACGGCGTTGCCGGCTCT
AATTGTTACAGCCCTCTGCAGAGCTACGGCTTCCGTCCTACCTATGGCGTGGGCCACCAGCCATACAGAGTCGTC
GTGCTGTCTTTCGAGCTGCTGCATGCTCCTGCTACAGTGTGCGGGCCCAAGAAATCCACCAACCTGGTGAAAAAC
AAGTGCGTAAATTTCGGCTCCGGCTCCGGATCTAGAGTGCAACCAACAGAGTCCATCGTCAGGTTTCCTAATATC
ACCAACCTGTGCCCTTTCGACGAAGTATTCAATGCTACCAGATTCGCCTCTGTGTACGCCTGGAACAGAAAGAGA
ATCTCTAACTGCGTGGCCGACTACAGTGTGCTGTATAACTTCGCTCCTTTCTTTGCTTTTAAATGCTATGGCGTG
TCTCCTACCAAGCTGAATGATCTGTGCTTCACCAACGTGTACGCCGACTCCTTTGTCATCAGAGGCAACGAAGTG
TCACAGATCGCCCCCGGACAAACCGGCAATATCGCCGATTACAACTATAAACTGCCTGATGACTTCACTGGATGT
GTGATCGCTTGGAACTCTAACAAGCTTGACTCCAAGGTGGGCGGCAACTACAACTACAGATACAGACTGTTCCGG
AAATCCAACCTGAAACCCTTTGAGAGGGATATCTCAACCGAGATCTACCAGGCCGGAAACAAGCCTTGCAATGGC
GTGGCTGGTGTGAACTGCTACTTCCCACTCCAGTCTTATGGCTTCAGACCTACCTACGGAGTGGGCCATCAGCCT
TATCGCGTGGTCGTGCTGTCTTTCGAGCTGCTGCACGCCCCCGCTACAGTGTGTGGGCCCAAGAAGTCTACCAAC
CTCGTGAAGAACAAGTGCGTCAACTTCCATCACCACCATCACCACCATCACTGA
SARS-CoV-2 Omicron Subvariants RBDs amino acids
(FIG. 8D;(SEQ ID NO. 10)
MFVFLVLLPLVSSQCVRVQPTESIVRFPNITNLCPFDEVFNATKFASVYAWNRKRISNCVADYSVLYNLAPFFTF
KCYGVSPTKLNDLCFTNVYADSFVIRGDEVRQIAPGQTGNIADYNYKLPDDFTGCVIAWNSNKLDSKVSGNYNYL
YRLFRKSNLKPFERDISTEIYQAGNKPCNGVAGFNCYFPLRSYSFRPTYGVGHQPYRVVVLSFELLHAPATVCGP
KKSTNLVKNKCVNFGSGSGSRVQPTESIVRFPNITNLCPFDEVFNATRFASVYAWNRKRISNCVADYSVLYNFAP
FFAFKCYGVSPTKLNDLCFTNVYADSFVIRGNEVSQIAPGQTGNIADYNYKLPDDFTGCVIAWNSNKLDSKVGGN
YNYLYRLFRKSNLKPFERDISTEIYQAGNKPCNGVAGFNCYFPLRSYGFRPTYGVGHQPYRVVVLSFELLHAPAT
VCGPKKSTNLVKNKCVNFGSGSSGRVQPTESIVRFPNITNLCPFHEVFNATTFASVYAWNRKRISNCVADYSVLY
NFAPFFAFKCYGVSPTKLNDLCFTNVYADSFVIRGNEVSQIAPGQTGNIADYNYKLPDDFTGCVIAWNSNKLDSK
VSGNYNYLYRLFRKSKLKPFERDISTEIYQAGNKPCNGVAGSNCYFPLQSYGFRPTYGVGHQPYRVVVLSFELLH
APATVCGPKKSTNLVKNKCVNFSGGSGSRVQPTESIVRFPNITNLCPFDEVFNATTFASVYAWNRKRISNCVADY
SVLYNFAPFFAFKCYGVSPTKLNDLCFTNVYADSFVIRGNEVSQIAPGQTGNIADYNYKLPDDFTGCVIAWNSNK
LDSTVGGNYNYRYRLFRKSKLKPFERDISTEIYQAGNKPCNGVAGVNCYFPLQSYGFRPTYGVGHQPYRVVVLSF
ELLHAPATVCGPKKSTNLVKNKCVNFGSSGGSRVQPTESIVRFPNITNLCPFHEVFNATTFASVYAWNRKRISNC
VADYSVIYNFAPFFAFKCYGVSPTKLNDLCFTNVYADSFVIRGNEVSQIAPGQTGNIADYNYKLPDDFTGCVIAW
NSNKLDSKPSGNYNYLYRLFRKSKLKPFERDISTEIYQAGNKPCNGVAGSNCYSPLQSYGFRPTYGVGHQPYRVV
VLSFELLHAPATVCGPKKSTNLVKNKCVNFGSGSGSRVQPTESIVRFPNITNLCPFDEVFNATRFASVYAWNRKR
ISNCVADYSVLYNFAPFFAFKCYGVSPTKLNDLCFTNVYADSFVIRGNEVSQIAPGQTGNIADYNYKLPDDFTGC
VIAWNSNKLDSKVGGNYNYRYRLFRKSNLKPFERDISTEIYQAGNKPCNGVAGVNCYFPLQSYGFRPTYGVGHQP
YRVVVLSFELLHAPATVCGPKKSTNLVKNKCVNFHHHHHHHH.
SARS-CoV-2 Omicron Subvariants RBDs-Fc cDNA
(FIG.8E; (SEQ ID NO. 11)
ATGTTTGTGTTTCTAGTGCTACTACCACTAGTGAGCAGCCAGTGCGTTAGAGTGCAGCCTACCGAAAGTATCGTG
AGATTTCCCAACATCACCAACCTGTGCCCATTCGACGAAGTGTTCAACGCTACAAAGTTCGCCTCTGTATACGCC
TGGAACAGAAAGCGGATCAGCAACTGCGTTGCCGACTACTCTGTTCTGTACAATCTGGCCCCTTTCTTCACCTTC
AAGTGTTACGGCGTTTCCCCCACCAAACTGAACGACCTGTGCTTCACTAACGTGTACGCTGATTCCTTCGTGATC
CGAGGCGACGAGGTGAGACAGATCGCCCCTGGCCAGACCGGAAACATAGCCGACTACAACTATAAGCTGCCGGAC
GACTTCACCGGCTGCGTGATCGCTTGGAATTCCAACAAATTGGACTCCAAGGTGTCCGGTAACTACAACTACCTG
TACAGACTGTTTAGAAAGTCCAATCTGAAACCCTTCGAAAGAGACATCTCCACCGAGATCTACCAGGCAGGCAAC
AAACCCTGCAACGGAGTGGCAGGCTTCAACTGCTACTTTCCTCTGCGTTCCTACTCTTTCAGACCAACCTACGGC
GTGGGCCACCAACCTTACCGCGTGGTGGTGCTGAGTTTCGAGCTGCTGCACGCTCCTGCCACAGTGTGCGGCCCT
AAGAAGTCTACAAACCTGGTCAAGAACAAGTGCGTGAATTTCGGCTCCGGCTCTGGCTCTAGAGTGCAGCCCACC
GAGTCTATCGTGAGATTCCCAAACATCACCAACTTGTGTCCTTTCGATGAGGTGTTCAACGCCACCAGATTCGCC
TCCGTGTACGCGTGGAACAGAAAGCGCATCAGTAATTGTGTGGCCGACTACTCCGTGCTGTACAACTTCGCCCCC
TTCTTCGCTTTCAAGTGCTACGGAGTATCTCCCACCAAACTGAACGACCTGTGCTTCACCAACGTGTACGCCGAC
TCCTTCGTGATCCGGGGAAACGAGGTTAGCCAGATTGCTCCGGGACAGACAGGGAACATCGCAGATTACAACTAC
AAGCTGCCCGACGACTTCACCGGCTGCGTGATCGCGTGGAACTCCAATAAGCTCGACTCCAAGGTCGGAGGCAAC
TATAACTACCTGTACAGACTGTTCCGGAAGTCCAATCTGAAGCCCTTCGAGAGAGACATCTCTACAGAGATCTAC
CAAGCTGGCAACAAGCCTTGCAACGGCGTGGCTGGTTTCAACTGCTACTTCCCCCTGCGGTCATACGGCTTCAGA
CCCACCTATGGTGTGGGCCACCAGCCTTATAGAGTGGTGGTGCTGAGCTTCGAGTTGCTGCATGCCCCTGCCACC
GTGTGTGGCCCCAAGAAGTCTACCAATCTGGTGAAGAACAAGTGTGTGAACTTCGGCTCAGGCAGCTCCGGCAGA
GTGCAGCCCACCGAGTCTATCGTGCGGTTCCCCAACATCACTAACCTGTGCCCCTTCCACGAGGTGTTCAACGCC
ACCACCTTCGCCTCCGTGTACGCCTGGAACCGGAAGAGAATCTCGAACTGCGTGGCCGACTACAGCGTCCTGTAC
AATTTTGCCCCTTTTTTTGCCTTCAAATGCTACGGCGTGTCCCCTACCAAGCTGAACGATCTGTGTTTCACAAAC
GTGTACGCTGATTCGTTCGTGATCCGGGGCAACGAAGTGTCGCAAATCGCACCTGGACAGACCGGCAACATCGCA
GACTACAACTACAAGCTGCCTGACGACTTCACGGGCTGTGTAATCGCCTGGAACTCCAACAAGCTGGATTCCAAG
GTGTCCGGCAATTACAACTACCTGTACCGGTTGTTCCGGAAGTCCAAGCTGAAGCCTTTCGAGAGAGATATCAGC
ACAGAGATCTATCAGGCCGGCAACAAGCCTTGTAATGGCGTCGCTGGCTCTAACTGCTACTTCCCTCTGCAGTCC
TACGGTTTCCGGCCAACCTACGGCGTGGGCCACCAGCCTTACAGAGTGGTCGTGTTGTCTTTCGAACTGCTGCAC
GCTCCTGCTACCGTGTGCGGCCCTAAGAAGTCCACCAACCTGGTGAAGAATAAGTGCGTCAATTTCTCCGGCGGT
TCCGGCTCTAGAGTGCAGCCTACAGAGTCCATCGTGAGATTCCCTAATATCACCAATCTGTGCCCTTTCGACGAG
GTGTTCAACGCTACCACATTCGCCTCTGTGTATGCTTGGAATAGAAAGCGGATCAGCAACTGCGTGGCCGACTAC
TCCGTGCTGTACAACTTCGCTCCTTTCTTCGCCTTCAAGTGCTACGGCGTGTCCCCTACCAAACTGAACGATCTG
TGCTTCACCAATGTGTACGCTGATTCTTTCGTGATAAGAGGCAACGAGGTCTCCCAGATCGCCCCTGGCCAGACC
GGCAACATAGCCGATTATAACTACAAGCTGCCCGACGATTTTACCGGCTGTGTCATCGCCTGGAACTCCAACAAG
CTGGATAGTACCGTGGGAGGCAACTACAACTACAGATACCGGCTGTTCAGAAAGTCTAAGCTGAAGCCATTCGAA
CGGGACATCTCTACCGAAATCTACCAGGCTGGCAACAAGCCTTGCAACGGCGTGGCCGGAGTCAACTGCTACTTC
CCTCTGCAGTCTTACGGATTCCGGCCTACTTACGGCGTGGGCCATCAGCCCTACAGAGTGGTTGTCCTGTCCTTT
GAACTGCTGCACGCCCCTGCGACCGTGTGCGGACCTAAGAAATCCACCAATCTCGTCAAAAACAAGTGCGTGAAC
TTTGGCAGCTCCGGCGGCTCTCGGGTGCAGCCTACCGAGTCCATCGTGCGGTTCCCTAACATCACAAACCTGTGT
CCTTTCCACGAGGTGTTCAATGCCACCACCTTTGCTTCTGTGTACGCATGGAACAGAAAGCGGATCTCCAACTGT
GTGGCCGACTACTCCGTGATCTACAACTTCGCCCCCTTCTTCGCTTTCAAGTGCTACGGGGTATCCCCCACAAAG
CTGAACGACCTGTGTTTTACCAATGTCTACGCTGACTCTTTTGTGATCCGGGGCAACGAGGTGTCTCAGATCGCT
CCTGGCCAGACCGGCAATATTGCCGACTACAACTACAAACTGCCTGACGACTTTACCGGATGTGTGATCGCCTGG
AACTCTAACAAACTGGACTCCAAACCTTCTGGCAACTACAACTACCTGTATCGGCTGTTCAGAAAGTCCAAGTTG
AAACCTTTCGAGCGGGACATCTCCACCGAAATCTACCAGGCTGGCAACAAACCTTGTAACGGCGTTGCCGGCTCT
AATTGTTACAGCCCTCTGCAGAGCTACGGCTTCCGTCCTACCTATGGCGTGGGCCACCAGCCATACAGAGTCGTC
GTGCTGTCTTTCGAGCTGCTGCATGCTCCTGCTACAGTGTGCGGGCCCAAGAAATCCACCAACCTGGTGAAAAAC
AAGTGCGTAAATTTCGGCTCCGGCTCCGGATCTAGAGTGCAACCAACAGAGTCCATCGTCAGGTTTCCTAATATC
ACCAACCTGTGCCCTTTCGACGAAGTATTCAATGCTACCAGATTCGCCTCTGTGTACGCCTGGAACAGAAAGAGA
ATCTCTAACTGCGTGGCCGACTACAGTGTGCTGTATAACTTCGCTCCTTTCTTTGCTTTTAAATGCTATGGCGTG
TCTCCTACCAAGCTGAATGATCTGTGCTTCACCAACGTGTACGCCGACTCCTTTGTCATCAGAGGCAACGAAGTG
TCACAGATCGCCCCCGGACAAACCGGCAATATCGCCGATTACAACTATAAACTGCCTGATGACTTCACTGGATGT
GTGATCGCTTGGAACTCTAACAAGCTTGACTCCAAGGTGGGCGGCAACTACAACTACAGATACAGACTGTTCCGG
AAATCCAACCTGAAACCCTTTGAGAGGGATATCTCAACCGAGATCTACCAGGCCGGAAACAAGCCTTGCAATGGC
GTGGCTGGTGTGAACTGCTACTTCCCACTCCAGTCTTATGGCTTCAGACCTACCTACGGAGTGGGCCATCAGCCT
TATCGCGTGGTCGTGCTGTCTTTCGAGCTGCTGCACGCCCCCGCTACAGTGTGTGGGCCCAAGAAGTCTACCAAC
CTCGTGAAGAACAAGTGCGTCAACTTCGGCTCTGGCGGAGGCGGCTCTGGTGGCGGTGGCTCTGGCTCCGAGCCT
AAGTCCTGCGACAAGACCCACACCTGTCCTCCTTGCCCAGCCCCCGAGCTGCTGGGCGGCCCTTCTGTGTTTCTG
TTCCCTCCAAAGCCCAAGGACACCCTGATGATCTCTCGGACCCCTGAAGTGACCTGCGTGGTGGTGGACGTGTCT
CACGAGGACCCTGAAGTGAAGTTCAACTGGTACGTGGACGGCGTGGAAGTGCACAACGCCAAAACCAAGCCTAGA
GAGGAACAGTACAACTCGACATACCGGGTTGTGTCCGTGCTCACTGTGCTCCACCAGGACTGGCTGAACGGCAAA
GAGTACAAGTGTGCCGTGTCTAACAAGGCTCTGCCCGCCCCTATCGAAAAGACCATCAGCAAGGCCAAGGGCCAA
CCTCGGGAACCTCAGGTGTACACCCTGCCCCCTAGCAGGGACGAGCTGACCAAGAACCAGGTTTCCCTGACATGC
CTGGTGAAGGGCTTCTACCCTTCGGACATCGCCGTGGAGTGGGAGTCCAACGGCCAGCCTGAGAACAACTACAAG
ACCACACCTCCTGTGCTCGACTCCGATGGCTCCTTTTTCCTGTACTCTAAGCTGACCGTGGATAAGAGTAGATGG
CAGCAGGGCAACGTGTTCTCATGCTCCGTGATGCACGAGGCCCTGCACAACCACTACACCCAAAAGTCTCTGAGT
CTGTCTCCTGGCAAGTGA
SARS-CoV-2 Omicron Subvariants RBDs-Fc Amino Acids
(FIG. 8E; SEQ ID NO. 12)
MFVFLVLLPLVSSQCVRVQPTESIVRFPNITNLCPFDEVFNATKFASVYAWNRKRISNCVADYSVLYNLAPFFTF
KCYGVSPTKLNDLCFTNVYADSFVIRGDEVRQIAPGQTGNIADYNYKLPDDFTGCVIAWNSNKLDSKVSGNYNYL
YRLFRKSNLKPFERDISTEIYQAGNKPCNGVAGFNCYFPLRSYSFRPTYGVGHQPYRVVVLSFELLHAPATVCGP
KKSTNLVKNKCVNFGSGSGSRVQPTESIVRFPNITNLCPFDEVFNATRFASVYAWNRKRISNCVADYSVLYNFAP
FFAFKCYGVSPTKLNDLCFTNVYADSFVIRGNEVSQIAPGQTGNIADYNYKLPDDFTGCVIAWNSNKLDSKVGGN
YNYLYRLFRKSNLKPFERDISTEIYQAGNKPCNGVAGFNCYFPLRSYGFRPTYGVGHQPYRVVVLSFELLHAPAT
VCGPKKSTNLVKNKCVNFGSGSSGRVQPTESIVRFPNITNLCPFHEVFNATTFASVYAWNRKRISNCVADYSVLY
NFAPFFAFKCYGVSPTKLNDLCFTNVYADSFVIRGNEVSQIAPGQTGNIADYNYKLPDDFTGCVIAWNSNKLDSK
VSGNYNYLYRLFRKSKLKPFERDISTEIYQAGNKPCNGVAGSNCYFPLQSYGFRPTYGVGHQPYRVVVLSFELLH
APATVCGPKKSTNLVKNKCVNFSGGSGSRVQPTESIVRFPNITNLCPFDEVFNATTFASVYAWNRKRISNCVADY
SVLYNFAPFFAFKCYGVSPTKLNDLCFTNVYADSFVIRGNEVSQIAPGQTGNIADYNYKLPDDFTGCVIAWNSNK
LDSTVGGNYNYRYRLFRKSKLKPFERDISTEIYQAGNKPCNGVAGVNCYFPLQSYGFRPTYGVGHQPYRVVVLSF
ELLHAPATVCGPKKSTNLVKNKCVNFGSSGGSRVQPTESIVRFPNITNLCPFHEVFNATTFASVYAWNRKRISNC
VADYSVIYNFAPFFAFKCYGVSPTKLNDLCFTNVYADSFVIRGNEVSQIAPGQTGNIADYNYKLPDDFTGCVIAW
NSNKLDSKPSGNYNYLYRLFRKSKLKPFERDISTEIYQAGNKPCNGVAGSNCYSPLQSYGFRPTYGVGHQPYRVV
VLSFELLHAPATVCGPKKSTNLVKNKCVNFGSGSGSRVQPTESIVRFPNITNLCPFDEVFNATRFASVYAWNRKR
ISNCVADYSVLYNFAPFFAFKCYGVSPTKLNDLCFTNVYADSFVIRGNEVSQIAPGQTGNIADYNYKLPDDFTGC
VIAWNSNKLDSKVGGNYNYRYRLFRKSNLKPFERDISTEIYQAGNKPCNGVAGVNCYFPLQSYGFRPTYGVGHQP
YRVVVLSFELLHAPATVCGPKKSTNLVKNKCVNFGSGGGGSGGGGSGSEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTVLHQDWLNGK
EYKCAVSNKALPAPIEKTISKAKGQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK.
CoV2-CoV1-MERS RBDs cDNA
(FIG. 14A; SEQ ID NO. 13)
ATGTTTGTGTTTCTAGTGCTACTACCACTAGTCTCAAGCCAGTGTGTGCGGGTGCAGCCAACAGAGAGCATCGTG
AGATTCCCTAACATCACCAACCTGTGCCCCTTTGGCGAGGTGTTCAACGCTACCAGATTCGCGAGCGTGTACGCC
TGGAACAGAAAGCGGATCTCCAATTGCGTGGCTGATTACTCCGTCCTGTACAACTCCGCCTCTTTTAGTACGTTC
AAGTGCTACGGAGTGAGTCCTACCAAGCTGAACGACCTGTGCTTCACCAATGTGTACGCGGACTCCTTCGTGATC
CGAGGCGACGAGGTGCGTCAGATCGCCCCTGGCCAGACCGGCAAAATCGCAGACTACAATTACAAGTTGCCCGAC
GACTTCACCGGATGTGTGATCGCCTGGAACTCCAACAACCTGGACTCTAAGGTGGGAGGCAACTACAACTACCTG
TACAGACTGTTCAGAAAGAGCAACCTGAAGCCTTTCGAGAGAGACATCTCCACAGAGATCTACCAGGCTGGATCA
ACCCCTTGTAATGGCGTGGAAGGCTTCAACTGCTACTTCCCACTGCAGTCCTACGGCTTCCAACCTACAAACGGA
GTGGGCTACCAGCCCTACAGAGTGGTGGTGCTTAGCTTCGAACTGTTGCATGCTCCAGCCACCGTGTGTGGCCCC
AAGAAGTCCACCAACCTGGTGAAGAACAAATGCGTGAACTTCGGCAGCGGCTCTGGCAGCCGCGTGCAGCCTACC
GAGTCTATCGTGCGGTTTCCCAACATCACGAACCTGTGCCCTTTCGGCGAGGTGTTCAACGCTACCCGGTTCGCT
AGTGTGTATGCCTGGAACCGGAAAAGAATCAGTAACTGTGTGGCCGATTACTCCGTGCTGTATAACTCCGCCTCC
TTCTCGACATTCAAGTGTTACGGGGTGTCTCCCACCAAGCTGAACGACCTGTGCTTTACAAACGTGTACGCCGAC
TCATTCGTGATCAGAGGTGATGAGGTACGGCAAATCGCTCCTGGCCAGACCGGCAAGATCGCCGATTACAACTAC
AAGCTGCCTGATGACTTTACAGGCTGTGTGATCGCTTGGAACTCCAACAACCTGGATTCCAAGGTGGGCGGCAAC
TACAACTACCTGTACAGGCTCTTCCGGAAGTCGAATCTGAAGCCATTCGAGCGGGACATCTCTACCGAGATCTAC
CAAGCCGGATCTACACCCTGCAACGGCGTGGAGGGCTTTAACTGCTACTTCCCCCTGCAGTCTTACGGCTTCCAG
CCCACTAATGGCGTCGGCTACCAGCCTTACCGGGTGGTTGTGCTCAGCTTTGAACTGTTGCACGCCCCCGCTACC
GTGTGCGGCCCTAAAAAGAGCACCAACCTGGTGAAAAACAAGTGCGTCAATTTCGGCTCCGGATCTTCCGGAAGA
GTAGTGCCCTCTGGCGATGTGGTGCGGTTCCCTAACATCACCAATCTGTGCCCCTTCGGCGAAGTGTTCAACGCC
ACCAAGTTCCCTTCAGTTTACGCTTGGGAACGGAAGAAGATCTCCAATTGCGTCGCTGACTACTCCGTGCTGTAC
AACTCCACCTTCTTCTCTACCTTTAAGTGCTATGGTGTGTCCGCGACCAAGCTGAATGACCTGTGCTTCTCCAAC
GTCTACGCTGATTCATTCGTGGTGAAGGGCGACGATGTGCGGCAGATCGCCCCTGGACAGACCGGCGTGATCGCG
GATTATAACTACAAGCTGCCTGACGACTTCATGGGCTGCGTGCTGGCCTGGAACACCAGAAACATCGACGCCACC
TCTACAGGCAACTACAACTACAAGTACAGATACCTGAGACACGGCAAGCTTAGACCTTTCGAGAGAGACATCTCT
AACGTTCCCTTCTCCCCTGACGGCAAGCCTTGCACCCCTCCTGCCCTGAACTGTTACTGGCCTCTGAACGACTAT
GGCTTCTACACCACCACGGGCATCGGCTACCAGCCTTACAGAGTGGTCGTGCTGTCTTTCGAACTGTTGAACGCC
CCTGCCACCGTGTGCGGCCCTAAGCTGAGCACCGACCTGATCAAGAACCAGTGTGTGAACTTTTCTGGCGGATCT
GGCTCCAGAGTGGTGCCGAGTGGCGATGTGGTTCGTTTTCCAAATATCACCAACCTGTGCCCCTTTGGTGAGGTA
TTCAACGCCACCAAGTTCCCTTCTGTGTACGCTTGGGAGAGAAAGAAGATCTCTAATTGCGTCGCCGACTACTCC
GTGCTCTATAACTCTACCTTCTTCTCCACCTTCAAGTGCTATGGCGTGAGCGCTACCAAACTGAACGATCTGTGT
TTCAGCAACGTGTACGCCGATTCCTTTGTGGTGAAGGGCGACGACGTGCGGCAAATCGCCCCAGGACAGACTGGC
GTGATCGCAGATTACAACTACAAACTCCCTGACGACTTCATGGGCTGCGTGTTAGCCTGGAACACCCGGAATATT
GACGCCACCTCCACCGGCAACTACAATTACAAGTACAGATACCTGCGGCATGGCAAGTTGAGACCCTTCGAGCGA
GATATCTCAAACGTACCATTCTCCCCTGATGGCAAGCCTTGCACACCCCCTGCTCTGAACTGCTACTGGCCTCTC
AACGACTACGGTTTCTACACCACAACAGGCATCGGCTATCAGCCTTATAGAGTTGTGGTGCTGTCCTTTGAGCTG
CTGAACGCTCCTGCCACCGTGTGCGGCCCTAAGCTGTCTACCGACCTGATCAAGAACCAGTGCGTGAACTTCGGC
TCTTCCGGCGGCTCAGAGGCCAAGCCATCCGGCTCCGTGGTCGAGCAAGCTGAGGGCGTTGAGTGCGACTTCTCT
CCTCTGCTCTCTGGCACCCCTCCCCAGGTGTACAACTTCAAGCGGCTGGTGTTCACCAACTGTAACTACAACCTG
ACCAAATTATTATCCCTGTTCTCAGTGAACGACTTCACGTGTTCCCAGATCTCTCCAGCCGCCATCGCCTCCAAC
TGCTACTCCTCTCTGATCCTGGATTACTTCTCTTATCCTCTGTCCATGAAGTCCGACCTGTCCGTGAGCTCCGCT
GGCCCCATCTCCCAGTTCAACTACAAGCAGTCGTTCTCCAACCCTACCTGCCTGATCCTGGCCACCGTCCCCCAC
AACCTGACCACAATCACCAAACCTCTAAAGTACTCCTACATTAACAAGTGCTCCAGACTGCTGTCCGACGACAGA
ACCGAGGTGCCTCAACTGGTGAACGCTAACCAGTATTCCCCCTGCGTGTCCATCGTGCCTTCGACAGTGTGGGAA
GATGGCGACTACTATCGGAAGCAGCTGTCTCCTCTGGAAGGCGGAGGCTGGCTGGTGGCTTCTGGCTCTACCGTG
GCCATGACCGAGCAGCTGCAGATGGGCTTCGGCATCACCGTGCAATACGGCACCGATACCAACTCCGTGTGTCCC
AAGCTGGAATTCGCCAACGACACCAAGATAGCTTCTCAGCTGGGCAACTGCGTGGAGTACGGCTCTGGCTCTGGC
TCCGAGGCCAAGCCTTCCGGCTCTGTGGTGGAGCAAGCCGAGGGCGTGGAATGCGACTTCTCCCCTCTGCTGTCA
GGCACCCCTCCTCAGGTCTACAACTTCAAGCGGCTGGTGTTCACCAATTGCAACTACAACCTGACCAAACTGCTG
TCTCTGTTTTCCGTGAACGACTTCACCTGTTCTCAGATCTCCCCCGCCGCTATCGCCTCTAACTGCTACTCCTCT
CTGATCCTGGATTACTTCTCCTACCCTCTGAGCATGAAATCTGACCTGAGCGTCTCGTCTGCCGGTCCTATCTCT
CAGTTTAACTACAAGCAGTCCTTTTCAAACCCCACATGTCTGATCCTGGCCACCGTGCCCCACAACCTGACCACA
ATTACCAAGCCTCTGAAGTATTCTTATATCAACAAGTGTTCCAGACTGCTGTCTGATGACAGAACAGAAGTGCCC
CAACTGGTGAATGCCAATCAGTATTCTCCCTGCGTGTCCATCGTGCCCTCTACCGTCTGGGAGGACGGAGATTAC
TACCGCAAGCAGCTGTCTCCTCTGGAAGGCGGAGGCTGGCTGGTTGCCTCTGGCTCCACCGTGGCTATGACCGAA
CAGCTCCAGATGGGCTTCGGAATCACCGTGCAGTACGGCACCGACACCAACAGTGTGTGTCCAAAGCTGGAGTTC
GCTAACGACACCAAGATCGCTTCCCAGCTGGGCAACTGTGTCGAATACCATCACCATCACCATCACCATCACTGA
CoV2-CoV1-MERS RBDs Amino Acids
(Figure 14A; SEQ ID NO. 14)
MFVFLVLLPLVSSQCVRVQPTESIVRFPNITNLCPFGEVENATRFASVYAWNRKRISNCVADYSVLYNSASFSTF
KCYGVSPTKLNDLCFTNVYADSFVIRGDEVRQIAPGQTGKIADYNYKLPDDFTGCVIAWNSNNLDSKVGGNYNYL
YRLFRKSNLKPFERDISTEIYQAGSTPCNGVEGENCYFPLQSYGFQPTNGVGYQPYRVVVLSFELLHAPATVCGP
KKSTNLVKNKCVNFGSGSGSRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPYRVVVLSFELLHAPAT
VCGPKKSTNLVKNKCVNFGSGSSGRVVPSGDVVRFPNITNLCPFGEVFNATKFPSVYAWERKKISNCVADYSVLY
NSTFFSTFKCYGVSATKLNDLCFSNVYADSFVVKGDDVRQIAPGQTGVIADYNYKLPDDFMGCVLAWNTRNIDAT
STGNYNYKYRYLRHGKLRPFERDISNVPFSPDGKPCTPPALNCYWPLNDYGFYTTTGIGYQPYRVVVLSFELLNA
PATVCGPKLSTDLIKNQCVNFSGGSGSRVVPSGDVVRFPNITNLCPFGEVFNATKFPSVYAWERKKISNCVADYS
VLYNSTFFSTFKCYGVSATKLNDLCFSNVYADSFVVKGDDVRQIAPGQTGVIADYNYKLPDDFMGCVLAWNTRNI
DATSTGNYNYKYRYLRHGKLRPFERDISNVPFSPDGKPCTPPALNCYWPLNDYGFYTTTGIGYQPYRVVVLSFEL
LNAPATVCGPKLSTDLIKNQCVNFGSSGGSEAKPSGSVVEQAEGVECDFSPLLSGTPPQVYNFKRLVFTNCNYNL
TKLLSLFSVNDFTCSQISPAAIASNCYSSLILDYFSYPLSMKSDLSVSSAGPISQFNYKQSFSNPTCLILATVPH
NLTTITKPLKYSYINKCSRLLSDDRTEVPQLVNANQYSPCVSIVPSTVWEDGDYYRKQLSPLEGGGWLVASGSTV
AMTEQLQMGFGITVQYGTDTNSVCPKLEFANDTKIASQLGNCVEYGSGSGSEAKPSGSVVEQAEGVECDFSPLLS
GTPPQVYNFKRLVFTNCNYNLTKLLSLFSVNDFTCSQISPAAIASNCYSSLILDYFSYPLSMKSDLSVSSAGPIS
QFNYKQSFSNPTCLILATVPHNLTTITKPLKYSYINKCSRLLSDDRTEVPQLVNANQYSPCVSIVPSTVWEDGDY
YRKQLSPLEGGGWLVASGSTVAMTEQLQMGFGITVQYGTDTNSVCPKLEFANDTKIASQLGNCVEYHHHHHHHH.
CoV2-CoV1-MERS RBDs-Fc cDNA
(Figure 14A; SEQ ID NO. 15)
ATGTTTGTGTTTCTAGTGCTACTACCACTAGTCTCAAGCCAGTGTGTGCGGGTGCAGCCAACAGAGAGCATCGTG
AGATTCCCTAACATCACCAACCTGTGCCCCTTTGGCGAGGTGTTCAACGCTACCAGATTCGCGAGCGTGTACGCC
TGGAACAGAAAGCGGATCTCCAATTGCGTGGCTGATTACTCCGTCCTGTACAACTCCGCCTCTTTTAGTACGTTC
AAGTGCTACGGAGTGAGTCCTACCAAGCTGAACGACCTGTGCTTCACCAATGTGTACGCGGACTCCTTCGTGATC
CGAGGCGACGAGGTGCGTCAGATCGCCCCTGGCCAGACCGGCAAAATCGCAGACTACAATTACAAGTTGCCCGAC
GACTTCACCGGATGTGTGATCGCCTGGAACTCCAACAACCTGGACTCTAAGGTGGGAGGCAACTACAACTACCTG
TACAGACTGTTCAGAAAGAGCAACCTGAAGCCTTTCGAGAGAGACATCTCCACAGAGATCTACCAGGCTGGATCA
ACCCCTTGTAATGGCGTGGAAGGCTTCAACTGCTACTTCCCACTGCAGTCCTACGGCTTCCAACCTACAAACGGA
GTGGGCTACCAGCCCTACAGAGTGGTGGTGCTTAGCTTCGAACTGTTGCATGCTCCAGCCACCGTGTGTGGCCCC
AAGAAGTCCACCAACCTGGTGAAGAACAAATGCGTGAACTTCGGCAGCGGCTCTGGCAGCCGCGTGCAGCCTACC
GAGTCTATCGTGCGGTTTCCCAACATCACGAACCTGTGCCCTTTCGGCGAGGTGTTCAACGCTACCCGGTTCGCT
AGTGTGTATGCCTGGAACCGGAAAAGAATCAGTAACTGTGTGGCCGATTACTCCGTGCTGTATAACTCCGCCTCC
TTCTCGACATTCAAGTGTTACGGGGTGTCTCCCACCAAGCTGAACGACCTGTGCTTTACAAACGTGTACGCCGAC
TCATTCGTGATCAGAGGTGATGAGGTACGGCAAATCGCTCCTGGCCAGACCGGCAAGATCGCCGATTACAACTAC
AAGCTGCCTGATGACTTTACAGGCTGTGTGATCGCTTGGAACTCCAACAACCTGGATTCCAAGGTGGGCGGCAAC
TACAACTACCTGTACAGGCTCTTCCGGAAGTCGAATCTGAAGCCATTCGAGCGGGACATCTCTACCGAGATCTAC
CAAGCCGGATCTACACCCTGCAACGGCGTGGAGGGCTTTAACTGCTACTTCCCCCTGCAGTCTTACGGCTTCCAG
CCCACTAATGGCGTCGGCTACCAGCCTTACCGGGTGGTTGTGCTCAGCTTTGAACTGTTGCACGCCCCCGCTACC
GTGTGCGGCCCTAAAAAGAGCACCAACCTGGTGAAAAACAAGTGCGTCAATTTCGGCTCCGGATCTTCCGGAAGA
GTAGTGCCCTCTGGCGATGTGGTGCGGTTCCCTAACATCACCAATCTGTGCCCCTTCGGCGAAGTGTTCAACGCC
ACCAAGTTCCCTTCAGTTTACGCTTGGGAACGGAAGAAGATCTCCAATTGCGTCGCTGACTACTCCGTGCTGTAC
AACTCCACCTTCTTCTCTACCTTTAAGTGCTATGGTGTGTCCGCGACCAAGCTGAATGACCTGTGCTTCTCCAAC
GTCTACGCTGATTCATTCGTGGTGAAGGGCGACGATGTGCGGCAGATCGCCCCTGGACAGACCGGCGTGATCGCG
GATTATAACTACAAGCTGCCTGACGACTTCATGGGCTGCGTGCTGGCCTGGAACACCAGAAACATCGACGCCACC
TCTACAGGCAACTACAACTACAAGTACAGATACCTGAGACACGGCAAGCTTAGACCTTTCGAGAGAGACATCTCT
AACGTTCCCTTCTCCCCTGACGGCAAGCCTTGCACCCCTCCTGCCCTGAACTGTTACTGGCCTCTGAACGACTAT
GGCTTCTACACCACCACGGGCATCGGCTACCAGCCTTACAGAGTGGTCGTGCTGTCTTTCGAACTGTTGAACGCC
CCTGCCACCGTGTGCGGCCCTAAGCTGAGCACCGACCTGATCAAGAACCAGTGTGTGAACTTTTCTGGCGGATCT
GGCTCCAGAGTGGTGCCGAGTGGCGATGTGGTTCGTTTTCCAAATATCACCAACCTGTGCCCCTTTGGTGAGGTA
TTCAACGCCACCAAGTTCCCTTCTGTGTACGCTTGGGAGAGAAAGAAGATCTCTAATTGCGTCGCCGACTACTCC
GTGCTCTATAACTCTACCTTCTTCTCCACCTTCAAGTGCTATGGCGTGAGCGCTACCAAACTGAACGATCTGTGT
TTCAGCAACGTGTACGCCGATTCCTTTGTGGTGAAGGGCGACGACGTGCGGCAAATCGCCCCAGGACAGACTGGC
GTGATCGCAGATTACAACTACAAACTCCCTGACGACTTCATGGGCTGCGTGTTAGCCTGGAACACCCGGAATATT
GACGCCACCTCCACCGGCAACTACAATTACAAGTACAGATACCTGCGGCATGGCAAGTTGAGACCCTTCGAGCGA
GATATCTCAAACGTACCATTCTCCCCTGATGGCAAGCCTTGCACACCCCCTGCTCTGAACTGCTACTGGCCTCTC
AACGACTACGGTTTCTACACCACAACAGGCATCGGCTATCAGCCTTATAGAGTTGTGGTGCTGTCCTTTGAGCTG
CTGAACGCTCCTGCCACCGTGTGCGGCCCTAAGCTGTCTACCGACCTGATCAAGAACCAGTGCGTGAACTTCGGC
TCTTCCGGCGGCTCAGAGGCCAAGCCATCCGGCTCCGTGGTCGAGCAAGCTGAGGGCGTTGAGTGCGACTTCTCT
CCTCTGCTCTCTGGCACCCCTCCCCAGGTGTACAACTTCAAGCGGCTGGTGTTCACCAACTGTAACTACAACCTG
ACCAAATTATTATCCCTGTTCTCAGTGAACGACTTCACGTGTTCCCAGATCTCTCCAGCCGCCATCGCCTCCAAC
TGCTACTCCTCTCTGATCCTGGATTACTTCTCTTATCCTCTGTCCATGAAGTCCGACCTGTCCGTGAGCTCCGCT
GGCCCCATCTCCCAGTTCAACTACAAGCAGTCGTTCTCCAACCCTACCTGCCTGATCCTGGCCACCGTCCCCCAC
AACCTGACCACAATCACCAAACCTCTAAAGTACTCCTACATTAACAAGTGCTCCAGACTGCTGTCCGACGACAGA
ACCGAGGTGCCTCAACTGGTGAACGCTAACCAGTATTCCCCCTGCGTGTCCATCGTGCCTTCGACAGTGTGGGAA
GATGGCGACTACTATCGGAAGCAGCTGTCTCCTCTGGAAGGCGGAGGCTGGCTGGTGGCTTCTGGCTCTACCGTG
GCCATGACCGAGCAGCTGCAGATGGGCTTCGGCATCACCGTGCAATACGGCACCGATACCAACTCCGTGTGTCCC
AAGCTGGAATTCGCCAACGACACCAAGATAGCTTCTCAGCTGGGCAACTGCGTGGAGTACGGCTCTGGCTCTGGC
TCCGAGGCCAAGCCTTCCGGCTCTGTGGTGGAGCAAGCCGAGGGCGTGGAATGCGACTTCTCCCCTCTGCTGTCA
GGCACCCCTCCTCAGGTCTACAACTTCAAGCGGCTGGTGTTCACCAATTGCAACTACAACCTGACCAAACTGCTG
TCTCTGTTTTCCGTGAACGACTTCACCTGTTCTCAGATCTCCCCCGCCGCTATCGCCTCTAACTGCTACTCCTCT
CTGATCCTGGATTACTTCTCCTACCCTCTGAGCATGAAATCTGACCTGAGCGTCTCGTCTGCCGGTCCTATCTCT
CAGTTTAACTACAAGCAGTCCTTTTCAAACCCCACATGTCTGATCCTGGCCACCGTGCCCCACAACCTGACCACA
ATTACCAAGCCTCTGAAGTATTCTTATATCAACAAGTGTTCCAGACTGCTGTCTGATGACAGAACAGAAGTGCCC
CAACTGGTGAATGCCAATCAGTATTCTCCCTGCGTGTCCATCGTGCCCTCTACCGTCTGGGAGGACGGAGATTAC
TACCGCAAGCAGCTGTCTCCTCTGGAAGGCGGAGGCTGGCTGGTTGCCTCTGGCTCCACCGTGGCTATGACCGAA
CAGCTCCAGATGGGCTTCGGAATCACCGTGCAGTACGGCACCGACACCAACAGTGTGTGTCCAAAGCTGGAGTTC
GCTAACGACACCAAGATCGCTTCCCAGCTGGGCAACTGTGTCGAATACGGCTCCGGCGGCGGCGGATCCGGCGGG
GGTGGGTCTGGTTCTGAACCTAAGTCTTGCGACAAGACCCACACCTGCCCTCCTTGCCCTGCTCCTGAACTGCTG
GGGGGCCATCCGTCTTTCTGTTCCCCCCTAAGCCTAAGGACACACTGATGATCTCTCGTACCCCTGAAGTGACCT
GCGTGGTGGTAGACGTGTCCCACGAGGATCCTGAGGTGAAGTTCAACTGGTACGTGGACGGCGTGGAAGTGCACA
ATGCTAAGACAAAGCCACGGGAAGAGCAGTACAACTCCACCTACAGAGTCGTGTCCGTGCTGACAGTGCTGCACC
AGGACTGGCTGAACGGAAAAGAGTACAAATGCGCCGTGTCCAACAAGGCTCTCCCTGCCCCAATCGAGAAGACCA
TCTCGAAGGCCAAGGGCCAGCCTAGAGAGCCTCAAGTGTACACCCTGCCTCCTTCTCGGGACGAGCTGACCAAGA
ACCAGGTCAGCCTGACTTGCCTTGTGAAGGGATTCTACCCTAGCGATATCGCCGTCGAGTGGGAGTCTAACGGCC
AGCCTGAGAACAACTACAAGACAACACCTCCTGTGCTGGACTCCGACGGCAGCTTCTTCCTGTACTCCAAGCTGA
CCGTCGACAAGTCCAGATGGCAGCAGGGAAACGTGTTCTCCTGCTCTGTGATGCACGAGGCTCTGCATAACCACT
ACACCCAGAAGTCTCTGTCCCTGAGCCCTGGCAAATGA
CoV2-CoV1-MERS RBDs-Fc Amino Acid
(Figure 14A; SEQ ID NO. 16)
MFVFLVLLPLVSSQCVRVQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVIAWNSNNLDSKVGGNYNYL
YRLFRKSNLKPFERDISTEIYQAGSTPCNGVEGENCYFPLQSYGFQPTNGVGYQPYRVVVLSFELLHAPATVCGP
KKSTNLVKNKCVNFGSGSGSRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPYRVVVLSFELLHAPAT
VCGPKKSTNLVKNKCVNFGSGSSGRVVPSGDVVRFPNITNLCPFGEVFNATKFPSVYAWERKKISNCVADYSVLY
NSTFFSTFKCYGVSATKLNDLCFSNVYADSFVVKGDDVRQIAPGQTGVIADYNYKLPDDFMGCVLAWNTRNIDAT
STGNYNYKYRYLRHGKLRPFERDISNVPFSPDGKPCTPPALNCYWPLNDYGFYTTTGIGYQPYRVVVLSFELLNA
PATVCGPKLSTDLIKNQCVNFSGGSGSRVVPSGDVVRFPNITNLCPFGEVFNATKFPSVYAWERKKISNCVADYS
VLYNSTFFSTFKCYGVSATKLNDLCFSNVYADSFVVKGDDVRQIAPGQTGVIADYNYKLPDDFMGCVLAWNTRNI
DATSTGNYNYKYRYLRHGKLRPFERDISNVPFSPDGKPCTPPALNCYWPLNDYGFYTTTGIGYQPYRVVVLSFEL
LNAPATVCGPKLSTDLIKNQCVNFGSSGGSEAKPSGSVVEQAEGVECDFSPLLSGTPPQVYNFKRLVFTNCNYNL
TKLLSLFSVNDFTCSQISPAAIASNCYSSLILDYFSYPLSMKSDLSVSSAGPISQFNYKQSFSNPTCLILATVPH
NLTTITKPLKYSYINKCSRLLSDDRTEVPQLVNANQYSPCVSIVPSTVWEDGDYYRKQLSPLEGGGWLVASGSTV
AMTEQLQMGFGITVQYGTDTNSVCPKLEFANDTKIASQLGNCVEYGSGSGSEAKPSGSVVEQAEGVECDFSPLLS
GTPPQVYNFKRLVFTNCNYNLTKLLSLFSVNDFTCSQISPAAIASNCYSSLILDYFSYPLSMKSDLSVSSAGPIS
QFNYKQSFSNPTCLILATVPHNLTTITKPLKYSYINKCSRLLSDDRTEVPQLVNANQYSPCVSIVPSTVWEDGDY
YRKQLSPLEGGGWLVASGSTVAMTEQLQMGFGITVQYGTDTNSVCPKLEFANDTKIASQLGNCVEYGSGGGGSGG
GGSGSEPKSCDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCAVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNH
YTQKSLSLSPGK.
Human IgG1-Fc cDNA
(SEQ ID NO. 17)
GAGCCTAAGTCCTGCGACAAGACCCACACCTGTCCTCCTTGCCCAGCCCCCGAGCTGCTGGGCGGCCCTTCTGTG
TTTCTGTTCCCTCCAAAGCCCAAGGACACCCTGATGATCTCTCGGACCCCTGAAGTGACCTGCGTGGTGGTGGAC
GTGTCTCACGAGGACCCTGAAGTGAAGTTCAACTGGTACGTGGACGGCGTGGAAGTGCACAACGCCAAAACCAAG
CCTAGAGAGGAACAGTACAACTCGACATACCGGGTTGTGTCCGTGCTCACTGTGCTCCACCAGGACTGGCTGAAC
GGCAAAGAGTACAAGTGTGCCGTGTCTAACAAGGCTCTGCCCGCCCCTATCGAAAAGACCATCAGCAAGGCCAAG
GGCCAACCTCGGGAACCTCAGGTGTACACCCTGCCCCCTAGCAGGGACGAGCTGACCAAGAACCAGGTTTCCCTG
ACATGCCTGGTGAAGGGCTTCTACCCTTCGGACATCGCCGTGGAGTGGGAGTCCAACGGCCAGCCTGAGAACAAC
TACAAGACCACACCTCCTGTGCTCGACTCCGATGGCTCCTTTTTCCTGTACTCTAAGCTGACCGTGGATAAGAGT
AGATGGCAGCAGGGCAACGTGTTCTCATGCTCCGTGATGCACGAGGCCCTGCACAACCACTACACCCAAAAGTCT
CTGAGTCTGTCTCCTGGCAAGTGA
Human IgG1-Fc Amino Acids
(SEQ ID NO. 18)
EPKSCDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCAVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNHYTQKS
LSLSPGK.

What is claimed:

1. A recombinant coronavirus protein comprising one or more tandemly arranged coronavirus receptor binding domains (RBDs) fused to an IgG Fc domain (RBDs-IgG Fc protein).

2. The RBDs-IgG Fc protein of claim 1, wherein the coronavirus is SARS-CoV-2, SARS-CoV-1, MERS or variants thereof.

3. The RBDs-IgG Fc protein of claim 2 where the variant is a SARS-CoV-2 variant.

4. The RBDs-IgG Fc protein of claim 1, further comprising one or more linker sequences.

5. The RBDs-IgG Fc protein of claim 1, wherein the IgG Fc domain is a human IgG Fc domain.

6. The RBDs-IgG Fc protein of claim 1, wherein the IgG Fc domain is a subclass 1 domain.

7. The RBDs-IgG Fc protein of claim 1, wherein the IgG Fc domain is a human IgG1 isotype having a K322A substitution.

8. The RBDs-IgG Fc protein of claim 1, comprising the amino acid sequence of SEQ ID NO. 2, 4, 6, 8, 10, 12 or 14, a sequence having at least 90%, 91%, 92%, 93%, 94%, 95%, 96%, 97%, 98%, 99%, 99.2%, 99.5%, 99.6%, 99.7%, 99.8%, 99.9%, 99.95%, 99.98% or 99.99% sequence identity thereto, or a fragment thereof.

9. The RBDs-IgG Fc protein of claim 4, wherein the linker sequence is a Gly-Ser peptide linker.

10. A nucleic acid molecule encoding for the RBDs-IgG Fc protein of claim 1.

11. The nucleic acid molecule of claim 10, wherein the nucleic acid is a cDNA sequence.

12. A recombinant expression vector comprising the nucleic acid molecule of claim 10.

13. The recombinant expression vector of claim 12, selected from the group consisting of a bacterial expression vector; and a eukaryotic expression vector.

14. The recombinant expression vector of claim 13, wherein the vector is a viral vector.

15. A pharmaceutical composition comprising the RBDs-IgG Fc protein of claim 1 and a pharmaceutically acceptable carrier.

16. A pharmaceutical composition comprising a nucleic acid encoding the RBDs-IgG Fc protein of claim 1 and a pharmaceutical acceptable carrier.

17. The RBDs-IgG Fc protein of claim 1, for use in the treatment or prevention of coronavirus infection.

18. The RBDs-IgG Fc protein of claim 17, wherein the coronavirus is SARS-CoV-2 or a variant thereof.

19. The nucleic acid molecule of claim 10, wherein the nucleic acid is an RNA for use in vaccination.

20. The nucleic acid molecule of claim 10, wherein the coronavirus is SARS-CoV-2 or a variant thereof.

21. A vaccine composition comprising the RBDs-IgG Fc protein of claim 1.

22. A vaccine composition comprising a nucleic acid molecule that encodes the RBDs-IgG Fc protein of claim 1.

23. The vaccine composition of claim 21 or 22, formulated for intranasal administration.

24. The vaccine composition of claim 21 or 22, wherein the vaccine composition is nanoparticle-based.

25. The vaccine composition of claim 24, wherein the nanoparticle is a nanoliposome.

26. A vaccine composition comprising nanoliposomes encapsulated with a nucleic acid molecule encoding for the RBDs-IgG Fc protein of claim 1.

27. A method for immunizing a subject comprising administering to such subject the vaccine composition of claim 21 or 22.

28. A method of treating or preventing clinical signs caused by coronavirus infection in a subject in need thereof, the method comprising administering to the subject a therapeutically effective amount of a vaccine composition of claim 21 or 22.

29. A vaccination kit comprising the vaccine composition of claim 21 or 22.

30. The vaccination kit according to claim 29, wherein the kit further comprises instructions for the administration of the vaccine composition to a subject.

31. A nanoparticle comprising the RBDs-IgG Fc protein of claim 1.

32. A nanoparticle comprising a nucleic acid encoding the RBDs-IgG Fc protein of claim 1.

33. The nanoparticle of claim 31 or 32, wherein the nanoparticle is a nanoliposome.

34. A recombinant coronavirus protein comprising one or more tandemly arranged coronavirus RBDs (RBDs protein).

35. The RBDs protein of claim 34, wherein the coronavirus is SARS-CoV-2, SARS-CoV-1, MERS or variants thereof.

36. The RBDs protein of claim 35, where the variant is a SARS-CoV-2 variant.

37. The RBDs protein of claim 34, further comprising one or more linker sequences.

38. The RBDs protein of claim 34, wherein the linker sequence is a Gly-Ser peptide linker.

39. A nucleic acid molecule encoding for the RBDs protein of claim 34.

40. The nucleic acid molecule of claim 39, wherein the nucleic acid is a cDNA sequence.

41. A recombinant expression vector comprising the nucleic acid molecule of claim 39.

42. The recombinant expression vector of claim 41, selected from the group consisting of a bacterial expression vector; and a eukaryotic expression vector.

43. The recombinant expression vector of claim 41, wherein the vector is a viral vector.

44. A pharmaceutical composition comprising the RBDs protein of claim 34 and a pharmaceutically acceptable carrier.

45. A pharmaceutical composition comprising a nucleic acid encoding the RBDs protein of claim 34 and a pharmaceutical acceptable carrier.

46. The RBDs protein of claim 34, for use in the treatment or prevention of coronavirus infection.

47. The RBDs protein of claim 46, wherein the coronavirus is SARS-CoV-2 or a variant thereof.

48. The nucleic acid molecule of claim 39, wherein the nucleic acid is an RNA for use in vaccination.

49. The nucleic acid molecule of claim 39, wherein the coronavirus is SARS-CoV-2 or a variant thereof.

50. A vaccine composition comprising the RBDs protein of claim 34.

51. A vaccine composition comprising a nucleic acid molecule that encodes the RBDs protein of claim 34.

52. The vaccine composition of claim 50 or 51, wherein the vaccine composition is formulated for systemic administration.

53. The vaccine composition of claim 50 or 51, wherein the vaccine composition is formulated for intramuscular administration.

54. The vaccine composition of claim 50 or 51, wherein the vaccine composition is a nanoparticle-based vaccine.

55. The vaccine composition of claim 54, wherein the nanoparticle is a nanoliposome.

56. A vaccine composition comprising nanoliposomes encapsulated with a nucleic acid molecule encoding for the RBDs protein of claim 34.

57. A method for immunizing a subject comprising administering to such subject the vaccine composition of claim 50 or 51.

58. A method of treating or preventing clinical signs caused by coronavirus infection in a subject in need thereof, the method comprising administering to the subject a therapeutically effective amount of a vaccine composition of claim 50 or 51.

59. A vaccination kit comprising the vaccine composition of claim 50 or 51.

60. The vaccination kit according to claim 59, wherein the kit further comprises instructions for the administration of the vaccine composition to a subject.

61. A nanoparticle comprising the RBDs-protein of claim 34.

62. A nanoparticle comprising a nucleic acid encoding the RBDs protein of claim 34.

63. The nanoparticle of claim 31 or 32, wherein the nanoparticle is a nanoliposome.


SEQUENCE LISTING (18 sequences)

SEQ ID NO: 1 - DNA, 4893 bp, synthetic construct
  misc_feature 1..4893: note = Synthetic: RBDs-Delta DNA
  source 1..4893: mol_type = other DNA
atgttcgtgtttctggtgctgctgcctctggtgtccagccagtgcgtgagggtgcagccaaccgagagcatcgtgcggttcccaaacatcacaaatctgtgccccttcggcgaggtgtttaacgccacccggtttgcttccgtgtacgcctggaacaggaagcggatctccaattgcgtggccgactacagcgtgctgtataattccgctagcttctctacatttaagtgctatggcgtgtctcccaccaagctgaacgatctgtgcttcacaaacgtgtacgccgactcctttgtgatcaggggagatgaagtgaggcagatcgctccaggacagaccggcaagatcgctgactacaactataagctgcccgacgatttcacaggctgcgtgatcgcttggaattctaacaatctggattccaaagtgggcggcaactacaattatagataccgcctgttcagaaagagcaacctgaagccttttgagcgcgacatcagcaccgagatctatcaggccggctctaagccatgcaacggcgtggagggcttcaattgttactttccactgcagtcttatggcttccagcccacaaacggcgtgggctaccagccttatagggtggtggtgctgagctttgagctgctgcacgctccagctaccgtgtgcggacccaagaagtctacaaacctggtgaagaataaatgcgtgaacttcggatccggaagcggctctagggtgcagcctaccgagtccatcgtgcggtttcctaatattacaaatctgtgcccattcggcgaagtctttaacgctacccggttcgccagcgtgtacgcttggaatagaaagcggatctctaactgcgtggctgactactccgtgctgtacaactccgcttctttttctacatttaaatgttatggcgtgtccccaactaaactgaatgacctgtgctttaccaacgtgtacgccgacagctttgtgatcagaggcgacgaagtcagacagatcgctcctggccagactggcaagatcgctgactataactacaagctgccagacgacttcaccggttgcgtgattgcttggaatagcaacaatctggattctaaagtgggcggcaactataattacagatatcgcctgttcagaaagtccaacctgaagccctttgagcgcgatatctctactgagatctatcaggctggcagcacaccttgtaatggtgtcgaaggttttaactgctattttccactgcagagctatggcttccagccaaccaacggagtcggctaccagccatatagagtggtggtgctgtcttttgagctgctgcatgctcctgctaccgtgtgcggaccaaagaagtccacaaatctggtgaagaacaagtgcgtgaatttcggatctggctcttccggaagagtccagccaactgaatccatcgtgcggtttcccaatattaccaatctgtgccctttcggcgaagtcttcaacgccacccgcttcgcctccgtgtacgcttggaacagaaaacggatctctaattgcgtggctgactactctgtgctgtacaactccgctagtttttctacatttaagtgttacggcgtgagccctactaaactgaatgatctgtgctttactaacgtgtacgctgactcttttgtgattagaggcgacgaagtccgccagattgccccaggacagaccggcacaattgccgattataactataagctgcctgacgacttcaccggatgcgtgattgcctggaactccaacaatctggatagtaaagtcggtggcaactacaattatctgtacaggctgttccgcaagtccaacctgaagccatttgagagggacatctctaccgagatctaccaggctggatccacaccatgcaacggagtgaaaggttttaactgctacttcccactgcagtcatacggcttccagccaacctacggagtgggctaccagccatacagagtcgtggtgctgagctttgagttactgcatgcccccgccaccgtgtgcggacctaagaagagcaccaatctggtcaaaaacaaatgcgtgaacttctctggaggatccggcagcagagtgcagccaaccgagtccatcgtgcgctttcctaatatcaccaacctgtgccccttcggcgaagtgtttaacgctaccagatttgctagcgtgtacgcatggaatagaaagcgcatttctaactgcgtggcagattatagcgtgctgtataattctgcttccttcagcacatttaaatgttacggcgtgtcccctactaaactgaacgacctgtgctttacaaacgtgtacgctgatagctttgtgatcagaggtgacgaagtccgccagatcgcccctggccagaccggcaacatcgctgactataattacaagctgcccgacgacttcaccgggtgcgtgattgcttggaacagcaataacctggacagcaaagtcggtggtaactataattacctgtatagactgttccgcaagtctaatctgaagccttttgagagagatatcagcaccgagatctaccaggccggctctacaccatgcaacggcgtgaagggcttcaattgttattttccactgcagtcctacggcttccagcccacctatggtgtcggctaccagccttatcgcgtcgtggtgctgtcttttgagttactgcatgcacctgccaccgtgtgcggccccaagaagtccaccaacctggtcaaaaataaatgcgtgaatttcggcagctctggaggctctagggtgcagccaactgaatctatcgtgcggtttcctaacattaccaacctgtgcccattcggcgaggtcttcaatgctacccgcttcgcatccgtgtacgcttggaaccgcaagcggatcagtaactgcgtggccgactactctgtgctgtataactctgcttccttcagcacctttaaatgttacggcgtgagcccaactaaactgaacgatctgtgctttacgaacgtgtacgccgactcttttgtgattcgcggcgacgaagtcaggcagattgctcctggccagacaggtaaaattgccgattacaattataagctgccagatgatttcacaggttgcgtgatcgcctggaactctaataacctggactcaaaagtcggaggtaattataactacctgtatcgcctgttccgtaagtctaatctgaagccctttgagagggacatctcaactgaaatttaccaggccggcagcacaccttgtaacggtgtcgaaggttttaattgctatttccctctgcagtcctacggttttcagcctacctatggcgtcggctaccagccataccgggtggtggtgctgtcctttgagctgctgcatgcccccgcaaccgtgtgcggcccaaagaagagcaccaacctggtcaagaacaaatgcgtgaattttggatctggctctggcagcagagtgcagcctaccgagtctatcgtgcgctttcccaatattactaacctgtgccctttcggcgaggtcttcaacgctaccaggtttgcatccgtgtacgcctggaatagaaagcgcatctctaactgcgtggcagactatagcgtgctgtacaattctgcttcttttagcacctttaaatgctacggcgtgtctcctactaaattaaacgacctgtgcttcactaacgtgtacgcagattcctttgtgatcagaggggacgaagtcaggcagatcgccccaggacagaccggaaaaattgccgactacaactacaagctgcctgatgatttcacaggatgcgtgattgcatggaactctaataatctggatagtaaagtgggtggtaattacaactatctgtacagactgttccgcaaatctaatctgaaaccatttgagagagatatttccactgaaatttatcaggccggcagcaccccttgcaacggagtcgaaggttttaactgttacttccctctgcagagctacggttttcagccaaccaatggcgtgggctaccagccatatcgcgttgtggtgctgagctttgagctactgcatgcccccgctaccgtgtgcggccctaagaagtctaccaacctggtgaaaaataagtgcgtgaattttggctctggaggaggaggatccggaggaggcggctctggctccgagccaaagtcttgcgacaagacccatacatgcccaccttgtccagctccagagctgctgggaggaccatccgtgttcctgtttccacccaagcccaaggataccctgatgatcagcaggaccccagaggtgacatgcgtggtggtggacgtgtctcacgaggatcccgaggtgaagtttaactggtacgtggacggcgtggaggtgcataatgctaagaccaagcctagggaggagcagtacaacagcacctatcgggtggtgtctgtgctgacagtgctgcaccaggattggctgaacggcaaggagtacaagtgcgccgtgtccaataaggccctgcctgctccaatcgagaagaccatcagcaaggctaagggccagcccagagagcctcaggtgtatacactgcctccatcccgcgacgagctgaccaagaaccaagtgagcctgacatgtctggtgaagggcttctaccccagcgatatcgccgtggagtgggagtctaatggccagcctgagaacaattataagaccacaccccctgtgctggactccgatggcagcttctttctgtactctaagctgaccgtggacaagtcccggtggcagcagggcaacgtgtttagctgctctgtgatgcatgaggctctgcacaatcattacacacagaagtccctgagcctgtctcctggcaagtga

SEQ ID NO: 2 - protein, 1630 aa, synthetic construct
  REGION 1..1630: note = Synthetic: RBDs-Delta amino acid
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFGSGSSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFSGGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSSGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGGGGSGGGGSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 4893 bp, synthetic construct
  misc_feature 1..4893: note = Synthetic: RBDs-Omicron DNA
  source 1..4893: mol_type = other DNA
atgtttgtgtttctagtgctactaccactagtttcctctcagtgcgtgcgggtgcagcccacagagtccatcgtgcgcttcccaaacatcaccaacctgtgtccattcggcgaagtgttcaatgccaccagatttgcctctgtctacgcctggaaccggaaaaggatctctaactgcgtggcggattactccgtgctgtacaactccgcctccttctctaccttcaagtgctacggcgtgagccctacaaagctgaacgacctgtgctttacaaacgtgtacgctgattccttcgtgatccggggcgacgaggtcagacagatcgcacctggccaaaccggcaagatcgccgattacaactacaagctgcctgatgacttcaccggctgtgtgatcgcctggaacagcaacaacctggactccaaggtgggcggcaactacaactacagatacagactgttcagaaagtccaacctgaagcctttcgaaagagacatctctactgaaatctaccaggccggctctaagccctgcaacggcgtggaaggcttcaactgttacttccctctgcagtcttatggcttccaacctaccaacggcgttggctaccagccttacagagtggtggtgctgtccttcgagctgctacatgcccctgctaccgtgtgtggccctaagaaatccacgaacctggttaagaacaagtgcgtgaactttggctccggatccggtagccgggtgcagcctaccgagtccattgtgcggttccccaacatcaccaacctgtgtcctttcgacgaggtgttcaacgctacccggtttgcttctgtgtacgcctggaaccggaagagaatcagcaattgtgtggcagattactccgtgctgtacaatctggccccttttttcaccttcaagtgctatggcgtgagccctaccaagctgaacgacttgtgctttacaaacgtgtacgccgattctttcgtgatcagaggcgacgaggtgagacaaatagcccccggccagaccggcaacatcgcagattacaactacaagctacctgacgacttcaccggctgcgtgatcgcctggaacagtaacaagctggactccaaggtgtcgggaaactacaactacctgtaccgcctgttcagaaagtccaacctgaaaccattcgagcgtgatatctctaccgagatctaccaggccggcaacaaaccttgtaacggcgtggccggatttaactgttactttccactgagatcctactctttccgcccaacttacggtgtgggccaccagccttacagggtcgtggtgctgtccttcgagctgctgcacgctcctgccaccgtgtgcggccccaagaaatctacaaatctggtgaaaaacaagtgcgtgaatttcggatctggatcgtctggcagagtgcagcctacagaatctatcgtgagattccctaacatcaccaacctgtgcccattcggcgaggtgttcaacgccacacggtttgcttctgtttacgcctggaacagaaagcggatctctaactgcgtggctgactactcggtgctgtacaactctgcttccttctctaccttcaagtgctacggtgtaagccctacaaagctgaacgacctgtgcttcaccaatgtgtacgccgactccttcgtgatcagaggggatgaagtgcggcagatcgctcctggacagaccggcaccatcgctgactacaactacaagctgccagatgacttcacgggctgcgtgatcgcctggaactccaacaacctggacagcaaggtggggggcaattacaattacctgtaccggctgttccgcaagagtaacctgaagcctttcgagagagacatcagtaccgagatctaccaggctggcagtaccccttgcaacggtgtgaagggctttaactgttacttccctctgcagtcctacggcttccaacccacttatggagtgggctaccagccctatcgggtggtggtactgagcttcgagctgctccatgctcctgccaccgtgtgcggcccaaagaagtcaaccaacctggtgaagaacaagtgtgtgaacttcagtggcggcagtggctcccgggtacagcctacagagagcatcgtgcgtttcccgaacatcaccaacctgtgccctttcggcgaggttttcaacgccacccggttcgcttctgtgtacgcctggaaccggaagagaatctctaactgcgtggccgattactcagtcttgtataactccgcgagcttctctaccttcaagtgctatggcgtgtcccccaccaagctgaatgacttgtgcttcaccaacgtgtacgccgactcctttgtgatccggggcgatgaagtgcgacagatcgcccctggccagaccggaaacatcgccgactacaactataagctgcctgacgacttcaccggctgtgtgatcgcctggaactccaacaacctggattcgaaggtgggcggcaactacaactatctgtacagactgttcagaaagtctaacctcaagccttttgaacgggacatctccaccgaaatctaccaggccggctccaccccctgtaacggcgtgaagggcttcaactgctacttccctctgcagtcctacggcttccagcccacctatggcgtaggctatcagccctacagggtggtagtgctgtccttcgagctgctgcacgctcccgcaaccgtgtgcggccctaagaagtccaccaacctggtcaagaataagtgcgtcaacttcggctcctctggcgggagccgtgtgcaacccacggaatcgattgtgcggttccctaacatcaccaacctgtgcccttttggtgaggttttcaacgccaccagattcgcttccgtgtacgcttggaacaggaagcggatctccaactgtgtcgccgactacagcgtgctgtacaactctgcttccttttctacctttaagtgctacggagtaagccctaccaagctgaacgacctgtgtttcaccaacgtgtacgccgattcttttgtgatcagaggagacgaggttagacagatcgctcctggccagaccggcaagatcgctgactacaattacaagctgcccgacgacttcaccggatgcgtgatcgcttggaattccaacaatctggactccaaggtgggcggcaactacaactacctgtaccggctgttcagaaagtccaatctgaagcccttcgagagagatatctccacagagatctaccaagctggctctaccccttgcaacggcgtggaaggcttcaactgctacttccccctgcagtcttacggcttccagcctacctacggcgtgggctaccaaccttacagagtcgtggtcctgagcttcgagctcctgcacgctcctgctacagtgtgcggacctaagaagtccacaaacctcgtgaagaacaagtgcgtgaacttcgggtccggctccggcagccgggtgcagcctaccgagtccatcgtccggttccccaacatcaccaacctgtgtccttttggcgaagtgtttaacgccacccggttcgcctccgtgtacgcttggaaccggaagagaatctccaattgcgtcgcggactacagcgtcctgtacaactccgcctctttctcgaccttcaaatgctacggcgtgtcccctacaaaactgaatgacctctgcttcacaaatgtgtacgctgactctttcgtgattagaggcgacgaggtacggcagatcgcccctggccagaccggcaagattgccgactacaactacaagctgcctgacgacttcaccggctgcgtgatcgcctggaactccaataacctggacagcaaggtgggaggcaattataattacctgtatagactcttcagaaagtctaacctgaagcccttcgaacgggacatctccacagaaatctaccaggctggttccaccccctgcaacggcgtggagggctttaactgctactttcctctgcaatcctacggctttcagcctaccaacggagtgggctaccagccttatagagtggttgtgctgtcctttgagctgctgcacgcccctgccacagtgtgcggccccaagaaatctaccaacctggtaaaaaacaaatgcgtgaacttcggctccgggggcggagggtcaggcggcggcggctccggctccgagcctaaatcctgcgataagacccacacctgtccaccttgccctgcccctgaactgctgggcgggccttctgtgttcctgttcccccctaagcctaaggacaccctgatgatctctagaacccctgaggtgacctgcgtggtagtggacgtgtctcatgaggaccccgaagtcaagttcaactggtacgtggatggcgtggaagtgcacaacgccaaaaccaaacctagagaggaacagtacaactccacctaccgggtggtgtccgtgctgaccgtgctccaccaggactggctgaacggaaaagagtacaagtgcgccgtgtctaataaggctctgccagctcctatcgagaagaccatctccaaggccaagggccagcctagagaacctcaggtctacaccctgcctccatctagagatgaactgaccaagaaccaggtgtccctgacctgcctggtgaagggcttctacccttctgacatcgctgtggaatgggagtccaacggacagcctgagaacaactacaagaccacgcctcctgtgctggattccgacggcagcttcttcctgtactccaagctgaccgtggacaaatccagatggcagcagggcaatgtgttctcctgctccgtgatgcacgaggccctgcacaaccactacacccagaagagcctgtctctgtctcctggtaaataa

SEQ ID NO: 4 - protein, 1630 aa, synthetic construct
  REGION 1..1630: note = Synthetic: RBDs-Omicron amino acid
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGSGSGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGSGSSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFSGGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSSGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGGGGSGGGGSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 4179 bp, synthetic construct
  misc_feature 1..4179: note = Synthetic: SARS-CoV-2 RBDs cDNA (Fig. 8 B)
  source 1..4179: mol_type = other DNA
atgttcgtgtttctggtgctgctgcctctggtgtccagccagtgcgtgagggtgcagccaaccgagagcatcgtgcggttcccaaacatcacaaatctgtgccccttcggcgaggtgtttaacgccacccggtttgcttccgtgtacgcctggaacaggaagcggatctccaattgcgtggccgactacagcgtgctgtataattccgctagcttctctacatttaagtgctatggcgtgtctcccaccaagctgaacgatctgtgcttcacaaacgtgtacgccgactcctttgtgatcaggggagatgaagtgaggcagatcgctccaggacagaccggcaagatcgctgactacaactataagctgcccgacgatttcacaggctgcgtgatcgcttggaattctaacaatctggattccaaagtgggcggcaactacaattatagataccgcctgttcagaaagagcaacctgaagccttttgagcgcgacatcagcaccgagatctatcaggccggctctaagccatgcaacggcgtggagggcttcaattgttactttccactgcagtcttatggcttccagcccacaaacggcgtgggctaccagccttatagggtggtggtgctgagctttgagctgctgcacgctccagctaccgtgtgcggacccaagaagtctacaaacctggtgaagaataaatgcgtgaacttcggatccggaagcggctctagggtgcagcctaccgagtccatcgtgcggtttcctaatattacaaatctgtgcccattcggcgaagtctttaacgctacccggttcgccagcgtgtacgcttggaatagaaagcggatctctaactgcgtggctgactactccgtgctgtacaactccgcttctttttctacatttaaatgttatggcgtgtccccaactaaactgaatgacctgtgctttaccaacgtgtacgccgacagctttgtgatcagaggcgacgaagtcagacagatcgctcctggccagactggcaagatcgctgactataactacaagctgccagacgacttcaccggttgcgtgattgcttggaatagcaacaatctggattctaaagtgggcggcaactataattacagatatcgcctgttcagaaagtccaacctgaagccctttgagcgcgatatctctactgagatctatcaggctggcagcacaccttgtaatggtgtcgaaggttttaactgctattttccactgcagagctatggcttccagccaaccaacggagtcggctaccagccatatagagtggtggtgctgtcttttgagctgctgcatgctcctgctaccgtgtgcggaccaaagaagtccacaaatctggtgaagaacaagtgcgtgaatttcggatctggctcttccggaagagtccagccaactgaatccatcgtgcggtttcccaatattaccaatctgtgccctttcggcgaagtcttcaacgccacccgcttcgcctccgtgtacgcttggaacagaaaacggatctctaattgcgtggctgactactctgtgctgtacaactccgctagtttttctacatttaagtgttacggcgtgagccctactaaactgaatgatctgtgctttactaacgtgtacgctgactcttttgtgattagaggcgacgaagtccgccagattgccccaggacagaccggcacaattgccgattataactataagctgcctgacgacttcaccggatgcgtgattgcctggaactccaacaatctggatagtaaagtcggtggcaactacaattatctgtacaggctgttccgcaagtccaacctgaagccatttgagagggacatctctaccgagatctaccaggctggatccacaccatgcaacggagtgaaaggttttaactgctacttcccactgcagtcatacggcttccagccaacctacggagtgggctaccagccatacagagtcgtggtgctgagctttgagttactgcatgcccccgccaccgtgtgcggacctaagaagagcaccaatctggtcaaaaacaaatgcgtgaacttctctggaggatccggcagcagagtgcagccaaccgagtccatcgtgcgctttcctaatatcaccaacctgtgccccttcggcgaagtgtttaacgctaccagatttgctagcgtgtacgcatggaatagaaagcgcatttctaactgcgtggcagattatagcgtgctgtataattctgcttccttcagcacatttaaatgttacggcgtgtcccctactaaactgaacgacctgtgctttacaaacgtgtacgctgatagctttgtgatcagaggtgacgaagtccgccagatcgcccctggccagaccggcaacatcgctgactataattacaagctgcccgacgacttcaccgggtgcgtgattgcttggaacagcaataacctggacagcaaagtcggtggtaactataattacctgtatagactgttccgcaagtctaatctgaagccttttgagagagatatcagcaccgagatctaccaggccggctctacaccatgcaacggcgtgaagggcttcaattgttattttccactgcagtcctacggcttccagcccacctatggtgtcggctaccagccttatcgcgtcgtggtgctgtcttttgagttactgcatgcacctgccaccgtgtgcggccccaagaagtccaccaacctggtcaaaaataaatgcgtgaatttcggcagctctggaggctctagggtgcagccaactgaatctatcgtgcggtttcctaacattaccaacctgtgcccattcggcgaggtcttcaatgctacccgcttcgcatccgtgtacgcttggaaccgcaagcggatcagtaactgcgtggccgactactctgtgctgtataactctgcttccttcagcacctttaaatgttacggcgtgagcccaactaaactgaacgatctgtgctttacgaacgtgtacgccgactcttttgtgattcgcggcgacgaagtcaggcagattgctcctggccagacaggtaaaattgccgattacaattataagctgccagatgatttcacaggttgcgtgatcgcctggaactctaataacctggactcaaaagtcggaggtaattataactacctgtatcgcctgttccgtaagtctaatctgaagccctttgagagggacatctcaactgaaatttaccaggccggcagcacaccttgtaacggtgtcgaaggttttaattgctatttccctctgcagtcctacggttttcagcctacctatggcgtcggctaccagccataccgggtggtggtgctgtcctttgagctgctgcatgcccccgcaaccgtgtgcggcccaaagaagagcaccaacctggtcaagaacaaatgcgtgaattttggatctggctctggcagcagagtgcagcctaccgagtctatcgtgcgctttcccaatattactaacctgtgccctttcggcgaggtcttcaacgctaccaggtttgcatccgtgtacgcctggaatagaaagcgcatctctaactgcgtggcagactatagcgtgctgtacaattctgcttcttttagcacctttaaatgctacggcgtgtctcctactaaattaaacgacctgtgcttcactaacgtgtacgcagattcctttgtgatcagaggggacgaagtcaggcagatcgccccaggacagaccggaaaaattgccgactacaactacaagctgcctgatgatttcacaggatgcgtgattgcatggaactctaataatctggatagtaaagtgggtggtaattacaactatctgtacagactgttccgcaaatctaatctgaaaccatttgagagagatatttccactgaaatttatcaggccggcagcaccccttgcaacggagtcgaaggttttaactgttacttccctctgcagagctacggttttcagccaaccaatggcgtgggctaccagccatatcgcgttgtggtgctgagctttgagctactgcatgcccccgctaccgtgtgcggccctaagaagtctaccaacctggtgaaaaataagtgcgtgaattttcatcaccaccatcaccaccatcactga

SEQ ID NO: 6 - protein, 1392 aa, synthetic construct
  REGION 1..1392: note = Synthetic: SARS-CoV-2 RBDs Amino Acid (FIG. 8B)
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFGSGSSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFSGGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSSGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHHHH

SEQ ID NO: 7 - DNA, 4893 bp, synthetic construct; identical to SEQ ID NO: 1
  misc_feature 1..4893: note = Synthetic: SARS-CoV-2 RBDs-Fc cDNA (FIG. 8C)
  source 1..4893: mol_type = other DNA

SEQ ID NO: 8 - protein, 1630 aa, synthetic construct; identical to SEQ ID NO: 2
  REGION 1..1630: note = Synthetic: SARS-CoV-2 RBDs-Fc amino acid (FIG. 8C)

SEQ ID NO: 9 - DNA, 4179 bp, synthetic construct
  misc_feature 1..4179: note = Synthetic: SARS-CoV-2 Omicron subvariants RBDs cDNA (FIG. 8D)
  source 1..4179: mol_type = other DNA
atgtttgtgtttctagtgctactaccactagtgagcagccagtgcgttagagtgcagcctaccgaaagtatcgtgagatttcccaacatcaccaacctgtgcccattcgacgaagtgttcaacgctacaaagttcgcctctgtatacgcctggaacagaaagcggatcagcaactgcgttgccgactactctgttctgtacaatctggcccctttcttcaccttcaagtgttacggcgtttcccccaccaaactgaacgacctgtgcttcactaacgtgtacgctgattccttcgtgatccgaggcgacgaggtgagacagatcgcccctggccagaccggaaacatagccgactacaactataagctgccggacgacttcaccggctgcgtgatcgcttggaattccaacaaattggactccaaggtgtccggtaactacaactacctgtacagactgtttagaaagtccaatctgaaacccttcgaaagagacatctccaccgagatctaccaggcaggcaacaaaccctgcaacggagtggcaggcttcaactgctactttcctctgcgttcctactctttcagaccaacctacggcgtgggccaccaaccttaccgcgtggtggtgctgagtttcgagctgctgcacgctcctgccacagtgtgcggccctaagaagtctacaaacctggtcaagaacaagtgcgtgaatttcggctccggctctggctctagagtgcagcccaccgagtctatcgtgagattcccaaacatcaccaacttgtgtcctttcgatgaggtgttcaacgccaccagattcgcctccgtgtacgcgtggaacagaaagcgcatcagtaattgtgtggccgactactccgtgctgtacaacttcgcccccttcttcgctttcaagtgctacggagtatctcccaccaaactgaacgacctgtgcttcaccaacgtgtacgccgactccttcgtgatccggggaaacgaggttagccagattgctccgggacagacagggaacatcgcagattacaactacaagctgcccgacgacttcaccggctgcgtgatcgcgtggaactccaataagctcgactccaaggtcggaggcaactataactacctgtacagactgttccggaagtccaatctgaagcccttcgagagagacatctctacagagatctaccaagctggcaacaagccttgcaacggcgtggctggtttcaactgctacttccccctgcggtcatacggcttcagacccacctatggtgtgggccaccagccttatagagtggtggtgctgagcttcgagttgctgcatgcccctgccaccgtgtgtggccccaagaagtctaccaatctggtgaagaacaagtgtgtgaacttcggctcaggcagctccggcagagtgcagcccaccgagtctatcgtgcggttccccaacatcactaacctgtgccccttccacgaggtgttcaacgccaccaccttcgcctccgtgtacgcctggaaccggaagagaatctcgaactgcgtggccgactacagcgtcctgtacaattttgcccctttttttgccttcaaatgctacggcgtgtcccctaccaagctgaacgatctgtgtttcacaaacgtgtacgctgattcgttcgtgatccggggcaacgaagtgtcgcaaatcgcacctggacagaccggcaacatcgcagactacaactacaagctgcctgacgacttcacgggctgtgtaatcgcctggaactccaacaagctggattccaaggtgtccggcaattacaactacctgtaccggttgttccggaagtccaagctgaagcctttcgagagagatatcagcacagagatctatcaggccggcaacaagccttgtaatggcgtcgctggctctaactgctacttccctctgcagtcctacggtttccggccaacctacggcgtgggccaccagccttacagagtggtcgtgttgtctttcgaactgctgcacgctcctgctaccgtgtgcggccctaagaagtccaccaacctggtgaagaataagtgcgtcaatttctccggcggttccggctctagagtgcagcctacagagtccatcgtgagattccctaatatcaccaatctgtgccctttcgacgaggtgttcaacgctaccacattcgcctctgtgtatgcttggaatagaaagcggatcagcaactgcgtggccgactactccgtgctgtacaacttcgctcctttcttcgccttcaagtgctacggcgtgtcccctaccaaactgaacgatctgtgcttcaccaatgtgtacgctgattctttcgtgataagaggcaacgaggtctcccagatcgcccctggccagaccggcaacatagccgattataactacaagctgcccgacgattttaccggctgtgtcatcgcctggaactccaacaagctggatagtaccgtgggaggcaactacaactacagataccggctgttcagaaagtctaagctgaagccattcgaacgggacatctctaccgaaatctaccaggctggcaacaagccttgcaacggcgtggccggagtcaactgctacttccctctgcagtcttacggattccggcctacttacggcgtgggccatcagccctacagagtggttgtcctgtcctttgaactgctgcacgcccctgcgaccgtgtgcggacctaagaaatccaccaatctcgtcaaaaacaagtgcgtgaactttggcagctccggcggctctcgggtgcagcctaccgagtccatcgtgcggttccctaacatcacaaacctgtgtcctttccacgaggtgttcaatgccaccacctttgcttctgtgtacgcatggaacagaaagcggatctccaactgtgtggccgactactccgtgatctacaacttcgcccccttcttcgctttcaagtgctacggggtatcccccacaaagctgaacgacctgtgttttaccaatgtctacgctgactcttttgtgatccggggcaacgaggtgtctcagatcgctcctggccagaccggcaatattgccgactacaactacaaactgcctgacgactttaccggatgtgtgatcgcctggaactctaacaaactggactccaaaccttctggcaactacaactacctgtatcggctgttcagaaagtccaagttgaaacctttcgagcgggacatctccaccgaaatctaccaggctggcaacaaaccttgtaacggcgttgccggctctaattgttacagccctctgcagagctacggcttccgtcctacctatggcgtgggccaccagccatacagagtcgtcgtgctgtctttcgagctgctgcatgctcctgctacagtgtgcgggcccaagaaatccaccaacctggtgaaaaacaagtgcgtaaatttcggctccggctccggatctagagtgcaaccaacagagtccatcgtcaggtttcctaatatcaccaacctgtgccctttcgacgaagtattcaatgctaccagattcgcctctgtgtacgcctggaacagaaagagaatctctaactgcgtggccgactacagtgtgctgtataacttcgctcctttctttgcttttaaatgctatggcgtgtctcctaccaagctgaatgatctgtgcttcaccaacgtgtacgccgactcctttgtcatcagaggcaacgaagtgtcacagatcgcccccggacaaaccggcaatatcgccgattacaactataaactgcctgatgacttcactggatgtgtgatcgcttggaactctaacaagcttgactccaaggtgggcggcaactacaactacagatacagactgttccggaaatccaacctgaaaccctttgagagggatatctcaaccgagatctaccaggccggaaacaagccttgcaatggcgtggctggtgtgaactgctacttcccactccagtcttatggcttcagacctacctacggagtgggccatcagccttatcgcgtggtcgtgctgtctttcgagctgctgcacgcccccgctacagtgtgtgggcccaagaagtctaccaacctcgtgaagaacaagtgcgtcaacttccatcaccaccatcaccaccatcactga

SEQ ID NO: 10 - protein, 1392 aa, synthetic construct
  REGION 1..1392: note = Synthetic: SARS-CoV-2 Omicron Subvariants RBDs amino acids (FIG. 8D)
MFVFLVLLPLVSSQCVRVQPTESIVRFPNITNLCPFDEVFNATKFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGSGSGSRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFGSGSSGRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNFSGGSGSRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFGSSGGSRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFGSGSGSRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFHHHHHHHH

SEQ ID NO: 11 - DNA, 4893 bp, synthetic construct
  misc_feature 1..4893: note = Synthetic: SARS-CoV-2 Omicron Subvariants RBDs-Fc cDNA (FIG.8E)
  source 1..4893: mol_type = other DNA
atgtttgtgtttctagtgctactaccactagtgagcagccagtgcgttagagtgcagcctaccgaaagtatcgtgagatttcccaacatcaccaacctgtgcccattcgacgaagtgttcaacgctacaaagttcgcctctgtatacgcctggaacagaaagcggatcagcaactgcgttgccgactactctgttctgtacaatctggcccctttcttcaccttcaagtgttacggcgtttcccccaccaaactgaacgacctgtgcttcactaacgtgtacgctgattccttcgtgatccgaggcgacgaggtgagacagatcgcccctggccagaccggaaacatagccgactacaactataagctgccggacgacttcaccggctgcgtgatcgcttggaattccaacaaattggactccaaggtgtccggtaactacaactacctgtacagactgtttagaaagtccaatctgaaacccttcgaaagagacatctccaccgagatctaccaggcaggcaacaaaccctgcaacggagtggcaggcttcaactgctactttcctctgcgttcctactctttcagaccaacctacggcgtgggccaccaaccttaccgcgtggtggtgctgagtttcgagctgctgcacgctcctgccacagtgtgcggccctaagaagtctacaaacctggtcaagaacaagtgcgtgaatttcggctccggctctggctctagagtgcagcccaccgagtctatcgtgagattcccaaacatcaccaacttgtgtcctttcgatgaggtgttcaacgccaccagattcgcctccgtgtacgcgtggaacagaaagcgcatcagtaattgtgtggccgactactccgtgctgtacaacttcgcccccttcttcgctttcaagtgctacggagtatctcccaccaaactgaacgacctgtgcttcaccaacgtgtacgccgactccttcgtgatccggggaaacgaggttagccagattgctccgggacagacagggaacatcgcagattacaactacaagctgcccgacgacttcaccggctgcgtgatcgcgtggaactccaataagctcgactccaaggtcggaggcaactataactacctgtacagactgttccggaagtccaatctgaagcccttcgagagagacatctctacagagatctaccaagctggcaacaagccttgcaacggcgtggctggtttcaactgctacttccccctgcggtcatacggcttcagacccacctatggtgtgggccaccagccttatagagtggtggtgctgagcttcgagttgctgcatgcccctgccaccgtgtgtggccccaagaagtctaccaatctggtgaagaacaagtgtgtgaacttcggctcaggcagctccggcagagtgcagcccaccgagtctatcgtgcggttccccaacatcactaacctgtgccccttccacgaggtgttcaacgccaccaccttcgcctccgtgtacgcctggaaccggaagagaatctcgaactgcgtggccgactacagcgtcctgtacaattttgcccctttttttgccttcaaatgctacggcgtgtcccctaccaagctgaacgatctgtgtttcacaaacgtgtacgctgattcgttcgtgatccggggcaacgaagtgtcgcaaatcgcacctggacagaccggcaacatcgcagactacaactacaagctgcctgacgacttcacgggctgtgtaatcgcctggaactccaacaagctggattccaaggtgtccggcaattacaactacctgtaccggttgttccggaagtccaagctgaagcctttcgagagagatatcagcacagagatctatcaggccggcaacaagccttgtaatggcgtcgctggctctaactgctacttccctctgcagtcctacggtttccggccaacctacggcgtgggccaccagccttacagagtggtcgtgttgtctttcgaactgctgcacgctcctgctaccgtgtgcggccctaagaagtccaccaacctggtgaagaataagtgcgtcaatttctccggcggttccggctctagagtgcagcctacagagtccatcgtgagattccctaatatcaccaatctgtgccctttcgacgaggtgttcaacgctaccacattcgcctctgtgtatgcttggaatagaaagcggatcagcaactgcgtggccgactactccgtgctgtacaacttcgctcctttcttcgccttcaagtgctacggcgtgtcccctaccaaactgaacgatctgtgcttcaccaatgtgtacgctgattctttcgtgataagaggcaacgaggtctcccagatcgcccctggccagaccggcaacatagccgattataactacaagctgcccgacgattttaccggctgtgtcatcgcctggaactccaacaagctggatagtaccgtgggaggcaactacaactacagataccggctgttcagaaagtctaagctgaagccattcgaacgggacatctctaccgaaatctaccaggctggcaacaagccttgcaacggcgtggccggagtcaactgctacttccctctgcagtcttacggattccggcctacttacggcgtgggccatcagccctacagagtggttgtcctgtcctttgaactgctgcacgcccctgcgaccgtgtgcggacctaagaaatccaccaatctcgtcaaaaacaagtgcgtgaactttggcagctccggcggctctcgggtgcagcctaccgagtccatcgtgcggttccctaacatcacaaacctgtgtcctttccacgaggtgttcaatgccaccacctttgcttctgtgtacgcatggaacagaaagcggatctccaactgtgtggccgactactccgtgatctacaacttcgcccccttcttcgctttcaagtgctacggggtatcccccacaaagctgaacgacctgtgttttaccaatgtctacgctgactcttttgtgatccggggcaacgaggtgtctcagatcgctcctggccagaccggcaatattgccgactacaactacaaactgcctgacgactttaccggatgtgtgatcgcctggaactctaacaaactggactccaaaccttctggcaactacaactacctgtatcggctgttcagaaagtccaagttgaaacctttcgagcgggacatctccaccgaaatctaccaggctggcaacaaaccttgtaacggcgttgccggctctaattgttacagccctctgcagagctacggcttccgtcctacctatggcgtgggccaccagccatacagagtcgtcgtgctgtctttcgagctgctgcatgctcctgctacagtgtgcgggcccaagaaatccaccaacctggtgaaaaacaagtgcgtaaatttcggctccggctccggatctagagtgcaaccaacagagtccatcgtcaggtttcctaatatcaccaacctgtgccctttcgacgaagtattcaatgctaccagattcgcctctgtgtacgcctggaacagaaagagaatctctaactgcgtggccgactacagtgtgctgtataacttcgctcctttctttgcttttaaatgctatggcgtgtctcctaccaagctgaatgatctgtgcttcaccaacgtgtacgccgactcctttgtcatcagaggcaacgaagtgtcacagatcgcccccggacaaaccggcaatatcgccgattacaactataaactgcctgatgacttcactggatgtgtgatcgcttggaactctaacaagcttgactccaaggtgggcggcaactacaactacagatacagactgttccggaaatccaacctgaaaccctttgagagggatatctcaaccgagatctaccaggccggaaacaagccttgcaatggcgtggctggtgtgaactgctacttcccactccagtcttatggcttcagacctacctacggagtgggccatcagccttatcgcgtggtcgtgctgtctttcgagctgctgcacgcccccgctacagtgtgtgggcccaagaagtctaccaacctcgtgaagaacaagtgcgtcaacttcggctctggcggaggcggctctggtggcggtggctctggctccgagcctaagtcctgcgacaagacccacacctgtcctccttgcccagcccccgagctgctgggcggcccttctgtgtttctgttccctccaaagcccaaggacaccctgatgatctctcggacccctgaagtgacctgcgtggtggtggacgtgtctcacgaggaccctgaagtgaagttcaactggtacgtggacggcgtggaagtgcacaacgccaaaaccaagcctagagaggaacagtacaactcgacataccgggttgtgtccgtgctcactgtgctccaccaggactggctgaacggcaaagagtacaagtgtgccgtgtctaacaaggctctgcccgcccctatcgaaaagaccatcagcaaggccaagggccaacctcgggaacctcaggtgtacaccctgccccctagcagggacgagctgaccaagaaccaggtttccctgacatgcctggtgaagggcttctacccttcggacatcgccgtggagtgggagtccaacggccagcctgagaacaactacaagaccacacctcctgtgctcgactccgatggctcctttttcctgtactctaagctgaccgtggataagagtagatggcagcagggcaacgtgttctcatgctccgtgatgcacgaggccctgcacaaccactacacccaaaagtctctgagtctgtctcctggcaagtga

SEQ ID NO: 12 - protein, 1630 aa, synthetic construct
  REGION 1..1630: note = Synthetic: SARS-CoV-2 Omicron Subvariants RBDs-Fc Amino Acids (FIG. 8E)
MFVFLVLLPLVSSQCVRVQPTESIVRFPNITNLCPFDEVFNATKFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFGSGSGSRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFGSGSSGRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNFSGGSGSRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFGSSGGSRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFGSGSGSRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFGSGGGGSGGGGSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 4275 bp, synthetic construct
  misc_feature 1..4275: note = Synthetic: CoV2-CoV1-MERS RBDs cDNA (FIG. 14A)
  source 1..4275: mol_type = other DNA
atgtttgtgtttctagtgctactaccactagtctcaagccagtgtgtgcgggtgcagccaacagagagcatcgtgagattccctaacatcaccaacctgtgcccctttggcgaggtgttcaacgctaccagattcgcgagcgtgtacgcctggaacagaaagcggatctccaattgcgtggctgattactccgtcctgtacaactccgcctcttttagtacgttcaagtgctacggagtgagtcctaccaagctgaacgacctgtgcttcaccaatgtgtacgcggactccttcgtgatccgaggcgacgaggtgcgtcagatcgcccctggccagaccggcaaaatcgcagactacaattacaagttgcccgacgacttcaccggatgtgtgatcgcctggaactccaacaacctggactctaaggtgggaggcaactacaactacctgtacagactgttcagaaagagcaacctgaagcctttcgagagagacatctccacagagatctaccaggctggatcaaccccttgtaatggcgtggaaggcttcaactgctacttcccactgcagtcctacggcttccaacctacaaacggagtgggctaccagccctacagagtggtggtgcttagcttcgaactgttgcatgctccagccaccgtgtgtggccccaagaagtccaccaacctggtgaagaacaaatgcgtgaacttcggcagcggctctggcagccgcgtgcagcctaccgagtctatcgtgcggtttcccaacatcacgaacctgtgccctttcggcgaggtgttcaacgctacccggttcgctagtgtgtatgcctggaaccggaaaagaatcagtaactgtgtggccgattactccgtgctgtataactccgcctccttctcgacattcaagtgttacggggtgtctcccaccaagctgaacgacctgtgctttacaaacgtgtacgccgactcattcgtgatcagaggtgatgaggtacggcaaatcgctcctggccagaccggcaagatcgccgattacaactacaagctgcctgatgactttacaggctgtgtgatcgcttggaactccaacaacctggattccaaggtgggcggcaactacaactacctgtacaggctcttccggaagtcgaatctgaagccattcgagcgggacatctctaccgagatctaccaagccggatctacaccctgcaacggcgtggagggctttaactgctacttccccctgcagtcttacggcttccagcccactaatggcgtcggctaccagccttaccgggtggttgtgctcagctttgaactgttgcacgcccccgctaccgtgtgcggccctaaaaagagcaccaacctggtgaaaaacaagtgcgtcaatttcggctccggatcttccggaagagtagtgccctctggcgatgtggtgcggttccctaacatcaccaatctgtgccccttcggcgaagtgttcaacgccaccaagttcccttcagtttacgcttgggaacggaagaagatctccaattgcgtcgctgactactccgtgctgtacaactccaccttcttctctacctttaagtgctatggtgtgtccgcgaccaagctgaatgacctgtgcttctccaacgtctacgctgattcattcgtggtgaagggcgacgatgtgcggcagatcgcccctggacagaccggcgtgatcgcggattataactacaagctgcctgacgacttcatgggctgcgtgctggcctggaacaccagaaacatcgacgccacctctacaggcaactacaactacaagtacagatacctgagacacggcaagcttagacctttcgagagagacatctctaacgttcccttctcccctgacggcaagccttgcacccctcctgccctgaactgttactggcctctgaacgactatggcttctacaccaccacgggcatcggctaccagccttacagagtggtcgtgctgtctttcgaactgttgaacgcccctgccaccgtgtgcggccctaagctgagcaccgacctgatcaagaaccagtgtgtgaacttttctggcggatctggctccagagtggtgccgagtggcgatgtggttcgttttccaaatatcaccaacctgtgcccctttggtgaggtattcaacgccaccaagttcccttctgtgtacgcttgggagagaaagaagatctctaattgcgtcgccgactactccgtgctctataactctaccttcttctccaccttcaagtgctatggcgtgagcgctaccaaactgaacgatctgtgtttcagcaacgtgtacgccgattcctttgtggtgaagggcgacgacgtgcggcaaatcgccccaggacagactggcgtgatcgcagattacaactacaaactccctgacgacttcatgggctgcgtgttagcctggaacacccggaatattgacgccacctccaccggcaactacaattacaagtacagatacctgcggcatggcaagttgagacccttcgagcgagatatctcaaacgtaccattctcccctgatggcaagccttgcacaccccctgctctgaactgctactggcctctcaacgactacggtttctacaccacaacaggcatcggctatcagccttatagagttgtggtgctgtcctttgagctgctgaacgctcctgccaccgtgtgcggccctaagctgtctaccgacctgatcaagaaccagtgcgtgaacttcggctcttccggcggctcagaggccaagccatccggctccgtggtcgagcaagctgagggcgttgagtgcgacttctctcctctgctctctggcacccctccccaggtgtacaacttcaagcggctggtgttcaccaactgtaactacaacctgaccaaattattatccctgttctcagtgaacgacttcacgtgttcccagatctctccagccgccatcgcctccaactgctactcctctctgatcctggattacttctcttatcctctgtccatgaagtccgacctgtccgtgagctccgctggccccatctcccagttcaactacaagcagtcgttctccaaccctacctgcctgatcctggccaccgtcccccacaacctgaccacaatcaccaaacctctaaagtactcctacattaacaagtgctccagactgctgtccgacgacagaaccgaggtgcctcaactggtgaacgctaaccagtattccccctgcgtgtccatcgtgccttcgacagtgtgggaagatggcgactactatcggaagcagctgtctcctctggaaggcggaggctggctggtggcttctggctctaccgtggccatgaccgagcagctgcagatgggcttcggcatcaccgtgcaatacggcaccgataccaactccgtgtgtcccaagctggaattcgccaacgacaccaagatagcttctcagctgggcaactgcgtggagtacggctctggctctggctccgaggccaagccttccggctctgtggtggagcaagccgagggcgtggaatgcgacttctcccctctgctgtcaggcacccctcctcaggtctacaacttcaagcggctggtgttcaccaattgcaactacaacctgaccaaactgctgtctctgttttccgtgaacgacttcacctgttctcagatctcccccgccgctatcgcctctaactgctactcctctctgatcctggattacttctcctaccctctgagcatgaaatctgacctgagcgtctcgtctgccggtcctatctctcagtttaactacaagcagtccttttcaaaccccacatgtctgatcctggccaccgtgccccacaacctgaccacaattaccaagcctctgaagtattcttatatcaacaagtgttccagactgctgtctgatgacagaacagaagtgccccaactggtgaatgccaatcagtattctccctgcgtgtccatcgtgccctctaccgtctgggaggacggagattactaccgcaagcagctgtctcctctggaaggcggaggctggctggttgcctctggctccaccgtggctatgaccgaacagctccagatgggcttcggaatcaccgtgcagtacggcaccgacaccaacagtgtgtgtccaaagctggagttcgctaacgacaccaagatcgcttcccagctgggcaactgtgtcgaataccatcaccatcaccatcaccatcactga

SEQ ID NO: 14 - protein, 1424 aa, synthetic construct
  REGION 1..1424: note = Synthetic: CoV2-CoV1-MERS RBDs Amino Acids (Figure 14A)
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSSGRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFSGGSGSRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFGSSGGSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYGSGSGSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYHHHHHHHH

SEQ ID NO: 15 - DNA, 4989 bp, synthetic construct
  misc_feature 1..4989: note = Synthetic: CoV2-CoV1-MERS RBDs-Fc cDNA (Figure 14A)
  source 1..4989: mol_type = other DNA
atgtttgtgtttctagtgctactaccactagtctcaagccagtgtgtgcgggtgcagccaacagagagcatcgtgagattccctaacatcaccaacctgtgcccctttggcgaggtgttcaacgctaccagattcgcgagcgtgtacgcctggaacagaaagcggatctccaattgcgtggctgattactccgtcctgtacaactccgcctcttttagtacgttcaagtgctacggagtgagtcctaccaagctgaacgacctgtgcttcaccaatgtgtacgcggactccttcgtgatccgaggcgacgaggtgcgtcagatcgcccctggccagaccggcaaaatcgcagactacaattacaagttgcccgacgacttcaccggatgtgtgatcgcctggaactccaacaacctggactctaaggtgggaggcaactacaactacctgtacagactgttcagaaagagcaacctgaagcctttcgagagagacatctccacagagatctaccaggctggatcaaccccttgtaatggcgtggaaggcttcaactgctacttcccactgcagtcctacggcttccaacctacaaacggagtgggctaccagccctacagagtggtggtgcttagcttcgaactgttgcatgctccagccaccgtgtgtggccccaagaagtccaccaacctggtgaagaacaaatgcgtgaacttcggcagcggctctggcagccgcgtgcagcctaccgagtctatcgtgcggtttcccaacatcacgaacctgtgccctttcggcgaggtgttcaacgctacccggttcgctagtgtgtatgcctggaaccggaaaagaatcagtaactgtgtggccgattactccgtgctgtataactccgcctccttctcgacattcaagtgttacggggtgtctcccaccaagctgaacgacctgtgctttacaaacgtgtacgccgactcattcgtgatcagaggtgatgaggtacggcaaatcgctcctggccagaccggcaagatcgccgattacaactacaagctgcctgatgactttacaggctgtgtgatcgcttggaactccaacaacctggattccaaggtgggcggcaactacaactacctgtacaggctcttccggaagtcgaatctgaagccattcgagcgggacatctctaccgagatctaccaagccggatctacaccctgcaacggcgtggagggctttaactgctacttccccctgcagtcttacggcttccagcccactaatggcgtcggctaccagccttaccgggtggttgtgctcagctttgaactgttgcacgcccccgctaccgtgtgcggccctaaaaagagcaccaacctggtgaaaaacaagtgcgtcaatttcggctccggatcttccggaagagtagtgccctctggcgatgtggtgcggttccctaacatcaccaatctgtgccccttcggcgaagtgttcaacgccaccaagttcccttcagtttacgcttgggaacggaagaagatctccaattgcgtcgctgactactccgtgctgtacaactccaccttcttctctacctttaagtgctatggtgtgtccgcgaccaagctgaatgacctgtgcttctccaacgtctacgctgattcattcgtggtgaagggcgacgatgtgcggcagatcgcccctggacagaccggcgtgatcgcggattataactacaagctgcctgacgacttcatgggctgcgtgctggcctggaacaccagaaacatcgacgccacctctacaggcaactacaactacaagtacagatacctgagacacggcaagcttagacctttcgagagagacatctctaacgttcccttctcccctgacggcaagccttgcacccctcctgccctgaactgttactggcctctgaacgactatggcttctacaccaccacgggcatcggctaccagccttacagagtggtcgtgctgtctttcgaactgttgaacgcccctgccaccgtgtgcggccctaagctgagcaccgacctgatcaagaaccagtgtgtgaacttttctggcggatctggctccagagtggtgccgagtggcgatgtggttcgttttccaaatatcaccaacctgtgcccctttggtgaggtattcaacgccaccaagttcccttctgtgtacgcttgggagagaaagaagatctctaattgcgtcgccgactactccgtgctctataactctaccttcttctccaccttcaagtgctatggcgtgagcgctaccaaactgaacgatctgtgtttcagcaacgtgtacgccgattcctttgtggtgaagggcgacgacgtgcggcaaatcgccccaggacagactggcgtgatcgcagattacaactacaaactccctgacgacttcatgggctgcgtgttagcctggaacacccggaatattgacgccacctccaccggcaactacaattacaagtacagatacctgcggcatggcaagttgagacccttcgagcgagatatctcaaacgtaccattctcccctgatggcaagccttgcacaccccctgctctgaactgctactggcctctcaacgactacggtttctacaccacaacaggcatcggctatcagccttatagagttgtggtgctgtcctttgagctgctgaacgctcctgccaccgtgtgcggccctaagctgtctaccgacctgatcaagaaccagtgcgtgaacttcggctcttccggcggctcagaggccaagccatccggctccgtggtcgagcaagctgagggcgttgagtgcgacttctctcctctgctctctggcacccctccccaggtgtacaacttcaagcggctggtgttcaccaactgtaactacaacctgaccaaattattatccctgttctcagtgaacgacttcacgtgttcccagatctctccagccgccatcgcctccaactgctactcctctctgatcctggattacttctcttatcctctgtccatgaagtccgacctgtccgtgagctccgctggccccatctcccagttcaactacaagcagtcgttctccaaccctacctgcctgatcctggccaccgtcccccacaacctgaccacaatcaccaaacctctaaagtactcctacattaacaagtgctccagactgctgtccgacgacagaaccgaggtgcctcaactggtgaacgctaaccagtattccccctgcgtgtccatcgtgccttcgacagtgtgggaagatggcgactactatcggaagcagctgtctcctctggaaggcggaggctggctggtggcttctggctctaccgtggccatgaccgagcagctgcagatgggcttcggcatcaccgtgcaatacggcaccgataccaactccgtgtgtcccaagctggaattcgccaacgacaccaagatagcttctcagctgggcaactgcgtggagtacggctctggctctggctccgaggccaagccttccggctctgtggtggagcaagccgagggcgtggaatgcgacttctcccctctgctgtcaggcacccctcctcaggtctacaacttcaagcggctggtgttcaccaattgcaactacaacctgaccaaactgctgtctctgttttccgtgaacgacttcacctgttctcagatctcccccgccgctatcgcctctaactgctactcctctctgatcctggattacttctcctaccctctgagcatgaaatctgacctgagcgtctcgtctgccggtcctatctctcagtttaactacaagcagtccttttcaaaccccacatgtctgatcctggccaccgtgccccacaacctgaccacaattaccaagcctctgaagtattcttatatcaacaagtgttccagactgctgtctgatgacagaacagaagtgccccaactggtgaatgccaatcagtattctccctgcgtgtccatcgtgccctctaccgtctgggaggacggagattactaccgcaagcagctgtctcctctggaaggcggaggctggctggttgcctctggctccaccgtggctatgaccgaacagctccagatgggcttcggaatcaccgtgcagtacggcaccgacaccaacagtgtgtgtccaaagctggagttcgctaacgacaccaagatcgcttcccagctgggcaactgtgtcgaatacggctccggcggcggcggatccggcgggggtgggtctggttctgaacctaagtcttgcgacaagacccacacctgccctccttgccctgctcctgaactgctgggcgggccatccgtctttctgttcccccctaagcctaaggacacactgatgatctctcgtacccctgaagtgacctgcgtggtggtagacgtgtcccacgaggatcctgaggtgaagttcaactggtacgtggacggcgtggaagtgcacaatgctaagacaaagccacgggaagagcagtacaactccacctacagagtcgtgtccgtgctgacagtgctgcaccaggactggctgaacggaaaagagtacaaatgcgccgtgtccaacaaggctctccctgccccaatcgagaagaccatctcgaaggccaagggccagcctagagagcctcaagtgtacaccctgcctccttctcgggacgagctgaccaagaaccaggtcagcctgacttgccttgtgaagggattctaccctagcgatatcgccgtcgagtgggagtctaacggccagcctgagaacaactacaagacaacacctcctgtgctggactccgacggcagcttcttcctgtactccaagctgaccgtcgacaagtccagatggcagcagggaaacgtgttctcctgctctgtgatgcacgaggctctgcataaccactacacccagaagtctctgtccctgagccctggcaaatga

SEQ ID NO: 16 - protein, 1662 aa, synthetic construct
  REGION 1..1662: note = Synthetic: CoV2-CoV1-MERS RBDs-Fc Amino Acid (Figure 14A)
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSSGRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFSGGSGSRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFGSSGGSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYGSGSGSEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYGSGGGGSGGGGSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 699 bp, Homo sapiens
  misc_feature 1..699: note = Human IgG1-Fc cDNA
  source 1..699: mol_type = genomic DNA
gagcctaagtcctgcgacaagacccacacctgtcctccttgcccagcccccgagctgctgggcggcccttctgtgtttctgttccctccaaagcccaaggacaccctgatgatctctcggacccctgaagtgacctgcgtggtggtggacgtgtctcacgaggaccctgaagtgaagttcaactggtacgtggacggcgtggaagtgcacaacgccaaaaccaagcctagagaggaacagtacaactcgacataccgggttgtgtccgtgctcactgtgctccaccaggactggctgaacggcaaagagtacaagtgtgccgtgtctaacaaggctctgcccgcccctatcgaaaagaccatcagcaaggccaagggccaacctcgggaacctcaggtgtacaccctgccccctagcagggacgagctgaccaagaaccaggtttccctgacatgcctggtgaagggcttctacccttcggacatcgccgtggagtgggagtccaacggccagcctgagaacaactacaagaccacacctcctgtgctcgactccgatggctcctttttcctgtactctaagctgaccgtggataagagtagatggcagcagggcaacgtgttctcatgctccgtgatgcacgaggccctgcacaaccactacacccaaaagtctctgagtctgtctcctggcaagtga

SEQ ID NO: 18 - protein, 697 aa, Homo sapiens
  REGION 1..697: note = misc_feature - Human IgG1-Fc Amino Acids
GLUPROLYSSERCYSASPLYSTHRHISTHRCYSPROPROCYSPROALAPROGLULEULEUGLYGLYPROSERVALPHELEUPHEPROPROLYSPROLYSASPTHRLEUMETILESERARGTHRPROGLUVALTHRCYSVALVALVALASPVALSERHISGLUASPPROGLUVALLYSPHEASNTRPTYRVALASPGLYVALGLUVALHISASNALALYSTHRLYSPROARGGLUGLUGLNTYRASNSERTHRTYRARGVALVALSERVALLEUTHRVALLEUHISGLNASPTRPLEUASNGLYLYSGLUTYRLYSCYSALAVALSERASNLYSALALEUPROALAPROILEGLULYSTHRILESERLYSALALYSGLYGLNPROARGGLUPROGLNVALTYRTHRLEUPROPROSERARGASPGLULEUTHRDLYSASNGLNVALSERLEUTHRCYSLEUVALLYSGLYPHETYRPROSERASPILEALAVALGLUTRPGLUSERASNGLYGLNPROGLUASNASNTYRLYSTHRTHRPROPROVALLEUASPSERASPGLYSERPHEPHELEUTYRSERLYSLEUTHRVALASPLYSSERARGTRPGLNGLNGLYASNVALPHESERCYSSERVALMETHISGLUALALEUHISASNHISTYRTHRGLNLYSSERLEUSERLEUSERPROGLYLYS